(19) United States
(12) Patent Application Publication
(10) Pub. No.: US 2004/0138245 A1
(43) Pub. Date: Jul. 15, 2004

(54) Product comprising mikanolide, dihydromikanolide or an analogue thereof combine with another anti-cancer agent for therapeutic use in cancer treatment

(76) Inventors: Gregoire Prevost, Antony (FR); Helene Coulomb, Igny (FR); Olivier Lavergne, Palaiseau (FR); Christophe Lanco, Dourdan (FR); Beng Poon Teng, Gif-Sur-Yvette (FR)

Correspondence Address: Muserlian, Lucas and Mercanti, 475 Park Avenue South, New York, NY, 10016, US

(21) Appl. No.: 10/478,387

(22) PCT Filed: May 29, 2002
(86) PCT No.: PCT/FR02/01800

(30) Foreign Application Priority Data
May 30, 2001 (FR) 01/07104

(51) Int. Cl. A61K 31/4745

(52) U.S. Cl. 514/283

(57) ABSTRACT

The invention concerns a product comprising at least mikanolide, dihydromikanolide or an analogue thereof combined with at least another anti-cancer agent for simultaneous, separate or prolonged therapeutic use in cancer treatment. In a preferred embodiment of the invention, the mikanolide, dihydromikanolide or one analogue thereof is combined with enzymatic inhibitors such as G heterotrimeric protein inhibitors or alkylating agents such as cis-platinum.

The present invention relates to a product comprising at least mikanolide, dihydromikanolide or their analogue in combination with at least one other anticancer agent for a therapeutic use which is simultaneous, separate or spread over time, in the treatment of cancer.

Currently, the development of new anticancer treatments largely involves the discovery of effective combinations of different therapeutic classes in order to enhance the antitumorous effect of each class and/or reduce the toxicity and extent of side effects.

Mikanolide and dihydromikanolide (see their structures in the figure below), can be obtained from extracts of Mikania plants, for example from the Mikania micrantha plant. Mikanolide and dihydromikanolide are sesquiterpenes of the germacrane family, i.e. having 4-isopropyl-1,7-dimethylcyclodecane as their hydrocarbon skeleton (Herz et al., Tetrahedron Lett. (1967) 3111-3115; Kiang et al., Phytochemistry (1968) 7: 1035-1037; Cuenca et al., J. Nat. Prod. (1988), 51, 625-626).

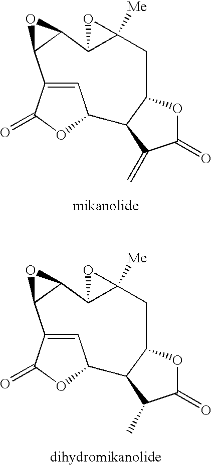

In PCT Patent Application WO 01/39720, the Applicant had itself already described the use of mikanolide and dihydromikanolide as an anticancer agent. The Applicant had also demonstrated that dihydromikanolide and mikanolide inhibit the replication of DNA by inhibiting the DNA polymerase enzymes necessary for the multiplication of eucaryotic and procaryotic cells as well as viruses.

More recently, it also described for the same use certain analogues of dihydromikanolide in Application no. PCT/FR02/00092. Said analogues correspond to general formula (I)

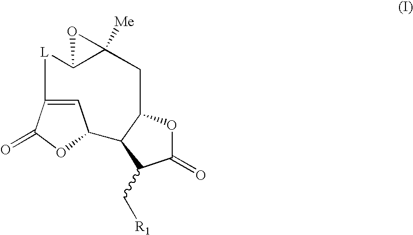

corresponding to general sub-formulae (I)₁ and (I)₂

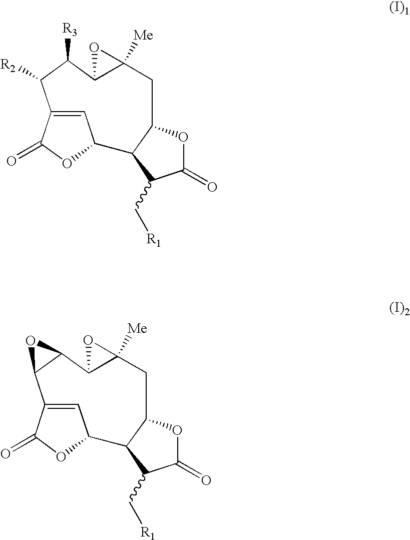

in which

R₁ represents a hydrogen atom or and SR₄ or NR₄R₅ radical;

R₂ represents SR₆ or NR₆R₇;

R₃ represents OH, O(CO)R₁₄, OSiR₁₅R₁₆R₁₇, O(CO)OR₁₈ or O(CO)NHR₁₈;

R₄ and R₆ represent, independently, an alkyl radical, a cycloalkyl, cycloalkylalkyl, hydroxyalkyl radical or also one of the aryl or aralkyl radicals optionally substituted on their aryl group by one or more radicals chosen from the alkyl, hydroxy or alkoxy radicals,

R₅ and R₇ represent, independently, a hydrogen atom, an alkyl radical, a cycloalkyl, cycloalkylalkyl, hydroxyalkyl radical or also one of the aryl or aralkyl radicals optionally substituted on their aryl group by one or more radicals chosen from the alkyl, hydroxy or alkoxy radicals,

R₄ and R₅ together with the nitrogen atom which carries them being able to form a heterocycle with 5 to 7 members, the additional members being chosen from the —CR₈R₉—, —NR₁₀—, —O— and —S— radicals, it being understood however that there can only be one member chosen from —O— or —S— in said heterocycle,

and R₆ and R₇ being able to form together with the nitrogen atom which carries them a heterocycle with 5 to 7 members, the additional members being chosen from the —CR₁₁R₁₂—, —NR₁₃-, —O— and —S— radicals, it being understood however that there can only be one member chosen from —O— or —S— in said heterocycle,

R₈, R₁₀, R₁₁, and R₁₃ represent, independently each time they occur, a hydrogen atom or an alkyl, alkoxycarbonyl or aralkyl radical,

R₉ and R₁₂ representing, independently each time they occur, a hydrogen atom or each of R₉ and R₁₂ being able to form with R₈ and R₁, respectively an —O—(CH₂)₂—O— radical attached on both sides to the carbon atom which carries them, such a radical only being present however once at most per NR₄R₅ or NR₆R₇ radical, represent, independently each time they occur, a hydrogen atom or an alkyl radical;

R₁₄ represents an alkyl, cycloalkyl or adamantyl radical or one of the aryl, heteroaryl, aralkyl or heteroaralkyl radicals optionally substituted on their aryl or heteroaryl group by one or more radicals chosen from a halogen atom and the alkyl, haloalkyl, nitro, hydroxy, alkoxy, alkylthio or phenyl radicals,

or also R₁₄ is such that R₁₄—COOH represents a natural amino acid or the optical enantiomer of such an amino acid;

R₁₅, R₁₆ and R₁₇ represent, independently, an alkyl radical or a phenyl radical;

R₁₈ represents an alkyl, cycloalkyl or adamantyl radical or one of the aryl, heteroaryl, aralkyl or heteroaralkyl radicals optionally substituted on their aryl or heteroaryl group by one or more radicals chosen from a halogen atom and the alkyl, haloalkyl, nitro, hydroxy, alkoxy, alkylthio or phenyl radicals;

it being understood however that when the compounds correspond to general sub-formula (I)₂, then R₁ does not represent a hydrogen atom;

or are salts of compounds corresponding to said general formula (I).

To date, no combination of anticancer compounds comprising mikanolide, dihydromikanolide or analogues of dihydromikanolide has been described. The Applicant has now discovered that these compounds used in combination with other anticancer agents provided a useful and even frequently synergistic anticancer activity.

A subject of the invention is therefore a product comprising at least mikanolide, dihydromikanolide or their analogue, optionally in the form of a pharmaceutically acceptable salt, in combination with at least one other anticancer agent for a therapeutic use which is simultaneous, separate or spread over time, in the treatment of the cancer.

According to the invention, the analogue of mikanolide or dihydromikanolide corresponds to general formula (I) as described above.

A compound of general formula (I) having at least one of the following characteristics is preferred:

the compound corresponds to general sub-formula (I)₁;

R₁ represents a hydrogen atom or an NR₄R₅ radical;

R₂ represents an NR₆R₇ radical;

R₃ represents OH or an O(CO)R₁₄, OSiR₁₅R₁₆R₁₇ or O(CO)NHR₁₈ radical.

More preferentially, a compound of general formula (I) is such that it has at least one of the following characteristics:

the compound corresponds to general sub-formula (I)₁;

R₁ represents a hydrogen atom;

R₂ represents an NR₆R₇ radical;

R₃ represents an O(CO)R₁₄, OSiR₁₅R₁₆R₁₇ or O(CO)NHR₁₈ radical.

Quite particularly, a compound of general formula (I) is such that it has at least one of the following characteristics:

the compound corresponds to general sub-formula (I)₁;

R₁ represents a hydrogen atom;

R₂ represents an NR₆R₇ radical and preferably an NR₆R₇ radical in which R₆ and R₇ are chosen independently from a hydrogen atom and an alkyl radical;

R₃ represents an O(CO)R₁₄, OSiR₁₅R₁₆R₁₇ or O(CO)NHR₁₈ radical.

Moreover, in the compounds of general formula (I), R₂ quite preferentially represents an NR₆R₇ radical in which R₆ and R₇ are alkyl radicals, and in particular an NR₆R₇ radical in which R₆ and R₇ are methyl radicals. R₃ quite preferentially represents an O(CO)NHR₁₈ radical.

Preferably also, in the compounds of general formula (I), R₄ represents an alkyl or aralkyl radical, and R₅ represents a hydrogen atom or an alkyl radical, or also R₄ and R₅ together with the nitrogen atom which carries them forms a heterocycle with 5 to 7 members, the additional members being chosen from the —CR₈R₉—, —NR₁₀—, —O— and —S-radicals. Preferably, R₈ represents, independently each time it occurs, a hydrogen atom or an alkyl radical (and preferably a hydrogen atom) and R₉ each time it occurs represents a hydrogen atom. Preferably, R₁₀ represents, independently each time it occurs, a hydrogen atom or an alkyl radical.

Preferably also, in the compounds of general formula (I), R₆ represents an alkyl or aralkyl radical, and R₇ represents a hydrogen atom or an alkyl radical, or also R₆ and R₇ together with the nitrogen atom which carries them form a heterocycle with 5 to 7 members, the additional members being chosen from the —CR₁₁R₁₂—, —NR₁₃—, —O— and —S— radicals. Preferably, R₁₁ represents, independently each time it occurs, a hydrogen atom or an alkyl or alkoxycarbonyl radical (and preferably a hydrogen atom) or also R₁₁ and R₁₂ once together represent an —O—(CH₂)₂—O— radical attached on both sides to the carbon atom which carries them. Preferably, R₁₃ represents, independently each time it occurs, a hydrogen atom or an alkyl radical.

Moreover, still in the compounds of the general formula, R₁₄ preferably represents an alkyl or cycloalkyl radical, or one of the aryl or heteroaryl radicals optionally substituted by a halogen atom or a haloalkyl or phenyl radical. More preferentially, R₁₄ represents a cycloalkyl radical or one of the aryl or heteroaryl radicals optionally substituted by a halogen atom or a haloalkyl radical. Even more preferentially, R₁₄ represents a cyclohexyl radical or one of the phenyl, thienyl or benzothienyl radicals optionally substituted by a halogen atom.

Moreover, the compounds from general formula (I) are preferably such that R₁₅, R₁₆ and R₁₇ represent alkyl radicals. Particularly preferentially, the compounds of general formula (I) are such that one of the R₁₅, R₁₆ and R₁₇ radicals represents a tert-butyl radical and the two others represent methyl radicals.

Finally, the compounds of general formula (I) are preferably such that R₁₈ represents an alkyl, cycloalkyl or adamantyl radical, or one of the aryl or heteroaryl radicals optionally substituted by a halogen atom or an alkyl, haloalkyl, alkoxy, alkylthio or phenyl radical. More preferentially, R₁₈ represents a cycloalkyl radical or one of the aryl or heteroaryl radicals optionally substituted by an alkyl, alkoxy or alkylthio radical. Even more preferably, R₁₈ represents one of the phenyl, thienyl or benzothienyl radicals optionally substituted by an alkyl, alkoxy or alkylthio radical.

Moreover, when R₄ and R₅ form together with the nitrogen atom which carries them a heterocycle with 5 to 7 members, the NR₄R₅ radical preferably represents one of the pyrrolyl, piperidyl, piperazinyl, morpholinyl or thiomorpholinyl radicals optionally substituted by an alkyl radical (which is preferably a methyl or ethyl radical, and more preferentially a methyl radical) on one of its carbon or nitrogen atoms, or by an —O—(CH₂)₂—O— radical attached on both sides to a carbon atom. More preferentially, when R₄ and R₅ together with the nitrogen atom which carries them form a heterocycle with 5 to 7 members, the NR₄R₅ radical represents one of the pyrrolyl, piperidyl, piperazinyl, morpholinyl or thiomorpholinyl radicals optionally substituted by an alkyl radical (which is preferably a methyl radical) on one of its carbon or nitrogen atoms.

Similarly, when & and R₇ together with the nitrogen atom which carries them form a heterocycle with 5 to 7 members, the NR₆R₇ radical preferably represents one of the pyrrolyl, piperidyl, piperazinyl, morpholinyl or thiomorpholinyl radicals optionally substituted by an alkyl radical (which is preferably a methyl or ethyl radical, and more preferentially a methyl radical) on one of its carbon or nitrogen atoms, or by an —O—(CH₂)₂—O— radical attached on both sides to a carbon atom. More preferentially, when R₆ and R₇ together with the nitrogen atom which carries them form a heterocycle with 5 to 7 members, the NR₆R₇ radical represents one of the pyrrolyl, piperidyl, piperazinyl, morpholinyl or thiomorpholinyl radicals optionally substituted by an alkyl radical (which is preferably a methyl radical) on one of its carbon or nitrogen atoms.

According to a particular variant of the invention, the analogue of mikanolide or dihydromikanolide corresponds to general formula (I)

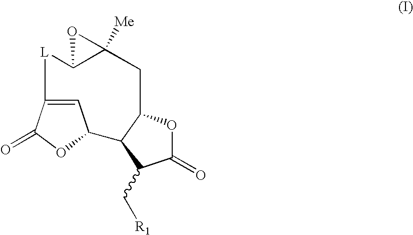

corresponding to general sub-formulae (I)₁ and (I)₂

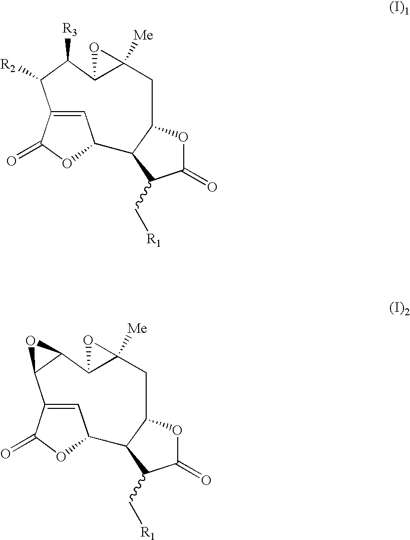

in which

R₁ represents a hydrogen atom or an SR₄ or NR₄R₅ radical;

R₂ represents SR₆ or NR₆R₇;

R₃ represents OH, O(CO)R₁₄, O(CO)OR₁₄, or OSiR₁₅R₁₆R₁₇;

R₄, R₅, R₆ and R₇ represent, independently, a hydrogen atom, an alkyl radical, a cycloalkyl, cycloalkylalkyl, hydroxyalkyl radical or also one of the aryl or aralkyl radicals optionally substituted on their aryl group by one or more radicals chosen from the alkyl, hydroxy or alkoxy radicals,

R₄ and R₅ being able to form together with the nitrogen atom which carries them a heterocycle with 5 to 7 members, the additional members being chosen from the —CR₈R₉—, —NR₁₀—, —O— and —S— radicals, it being understood however that there can only be one member chosen from —O— or —S— in said heterocycle,

and R₆ and R₇ being able to form together with the nitrogen atom which carries them a heterocycle with 5 to 7 members, the additional members being chosen from the —CR₁₁R₁₂—, —NR₁₃—, —O— and —S— radicals, it being understood however that there can only be one member chosen from —O— or —S— in said heterocycle,

R₈, R₁₀, R₁₁ and R₁₃ represent, independently each time they occur, a hydrogen atom or an alkyl, alkoxycarbonyl or aralkyl radical,

R₉ and R₁₂ representing, independently each time they occur, a hydrogen atom or each of R₉ and R₁₂ being able to form with R₈ and R₁₁ respectively an —O—(CH₂)₂—O— radical attached on both sides to the carbon atom which carries it, such a radical only being present however once at most per NR₄R₅ or NR₆R₇ radical,

represent, independently each time they occur, a hydrogen atom or an alkyl radical;

R₁₄, R₁₅, R₁₆ and R₁₇ represent, independently, a hydrogen atom, an alkyl radical or one of the aryl or aralkyl radicals optionally substituted on their aryl group by one or more radicals chosen from the alkyl, hydroxy or alkoxy radicals;

or are salts of compounds corresponding to said general formula (I).

According to the same variant of the invention, the analogue of mikanolide or dihydromikanolide corresponds to general formula (I)

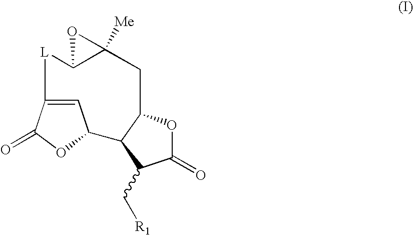

corresponding to general sub-formulae (I)₁ and (I)₂

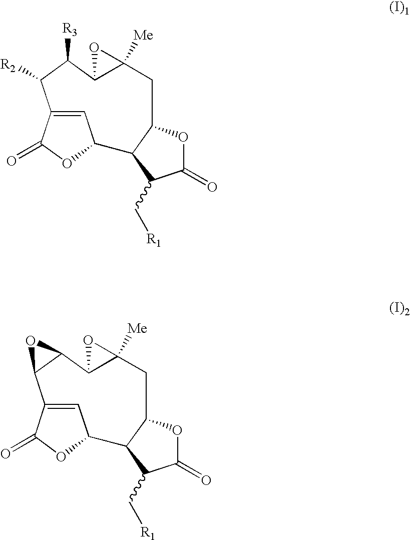

in which

R₁ represents a hydrogen atom or an SR₄ or NR₄R₅ radical;

R₂ represents SR₆ or NR₆R₇;

R₃ represents OH, O(CO)R₁₄, O(CO)OR₁₄, or OSiR₁₅R₁₆R₁₇;

R₄, R₅, R₆ and R₇ represent, independently, a hydrogen atom, an alkyl radical, a cycloalkyl, cycloalkylalkyl, hydroxyalkyl radical or also one of the aryl or aralkyl radicals optionally substituted on their aryl group by one or more radicals chosen from the alkyl, hydroxy or alkoxy radicals,

R₄ and R₅ being able to form together with the nitrogen atom which carries them a heterocycle with 5 to 7 members, the additional members being chosen from the —CR₈R₉—, —NR₁₀—, —O— and —S— radicals, it being understood however that there can only be one member chosen from —O— or —S— in said heterocycle,

and R₆ and R₇ being able to form together with the nitrogen atom which carries them a heterocycle with 5 to 7 members, the additional members being chosen from the —CR₁₁R₁₂—, —NR₁₃—, —O— and —S— radicals, it being understood however that there can only be one member chosen from —O— or —S— in said heterocycle,

R₈, R₁₀, R₁₁ and R₁₃ represent, independently each time they occur, a hydrogen atom or an alkyl, alkoxycarbonyl or aralkyl radical,

R₉ and R₁₂ representing, independently each time they occur, a hydrogen atom or each of R₉ and R₁₂ being able to form with R₈ and R., respectively an —O—(CH₂)₂—O— radical attached on both sides to the carbon atom which carries them, such a radical only being present however once at most per NR₄R₅ or NR₆R₇ radical,

represent, independently each time they occur, a hydrogen atom or an alkyl radical;

R₁₄, R₁₅, R₁₆ and R₁₇ represent, independently, a hydrogen atom, an alkyl radical or one of the aryl or aralkyl radicals optionally substituted on their aryl group by one or more radicals chosen from the alkyl, hydroxy or alkoxy radicals;

or is a pharmaceutically acceptable salt of a compound of general formula (I).

By alkyl or lower alkyl, unless specified otherwise, is meant in the present application a linear or branched alkyl radical containing 1 to 12 carbon atoms, and preferably 1 to 6 carbon atoms. By cycloalkyl, unless otherwise specified, is meant in the present Application a monocyclic carbon system containing 3 to 7 carbon atoms. By haloalkyl, is meant in the present application an alkyl radical at least one of the hydrogen atoms of which (and optionally all) is replaced by a halogen atom. By carbocyclic or heterocyclic aryl, unless specified otherwise, is meant in the present application a carbocyclic or heterocyclic system comprising one to three condensed rings at least one of which is an aromatic ring, a system being referred to as heterocyclic when at least one of the rings which compose it comprises one or more heteroatoms (O, N or S). By aryl, unless specified otherwise, is meant in the present application a carbocyclic aryl radical. By heteroaryl, is meant in the present application a heterocyclic aryl radical. By heterocycle, unless otherwise specified is meant in the present application a non aromatic heterocycle comprising 3 to 7 members (and preferably 5 to 7 members) the heteroatoms of which are chosen from the nitrogen, oxygen and sulphur atoms. By haloalkyl, is meant in the present application an alkyl radical at least one of the hydrogen atoms of which (and optionally all) is replaced by a halogen atom. Finally, by halogen atom, is meant in the present application the fluorine, chlorine, bromine or iodine atoms.

By alkoxy, haloalkoxy, hydroxyalkyl, cycloalkylalkyl, aralkyl and heteroaralkyl radicals, is meant respectively in the present application the alkoxy, haloalkoxy, hydroxyalkyl, cycloalkylalkyl and aralkyl radicals the alkyl, haloalkyl, cycloalkyl, aryl and heteroaralkyl radicals of which have the meanings indicated previously.

By natural amino acid, is meant valine (Val), leucine (Leu), isoleucine (Ile), methionine (Met), phenylalanine (Phe), asparagine (Asn), glutamic acid (Glu), glutamine (Gln), histidine (His), lysine (Lys), arginine (Arg), aspartic acid (Asp), glycine (Gly), alanine (Ala), serine (Ser), threonine (Thr), tyrosine (Tyr), tryptophan (Trp), cysteine (Cys) or proline (Pro).

By linear or branched alkyl having 1 to 6 carbon atoms, is meant in the present application in particular the methyl, ethyl, propyl, isopropyl, butyl, isobutyl, sec-butyl and tert-butyl, pentyl, neopentyl, isopentyl, hexyl, isohexyl radicals. By alkoxy, is meant in the present application in particular the methoxy, ethoxy and isopropoxy radicals, and in particular the methoxy and ethoxy radicals. By cycloalkyl, is meant in the present application in particular the cyclopropyl, cyclobutyl, cyclopentyl and cyclohexyl radicals. By haloalkyl, is meant in the present application in particular the trifluoromethyl radical. By haloalkoxy is meant in the present application in particular the trifluoromethyl radical. By carbocyclic aryl, is meant in the present application in particular the phenyl, naphthyl and phenanthryl radicals, preferably the phenyl and naphthyl radicals and more preferentially the phenyl radical. By heterocyclic aryl, is meant in the present application in particular the pyrrolyl, furanyl, benzofuranyl, thienyl, benzothienyl, pyridyl, pyrimidinyl, triazinyl, imidazolyl, oxazolyl, thiazolyl, indolyl and quinolyl radicals, and preferably the furanyl, benzofuranyl, thienyl and benzothienyl radicals. By aralkyl, is meant in the present application in particular a phenalkyl radical, and preferably the benzyl radical. By heteroaralkyl, is meant in the present application in particular a thienylalkyl, furanylalkyl, pyrrolylalkyl and thiazolylalkyl radical (the alkyl radical of said radicals preferably being a methyl radical), and preferably a thienylalkyl radical (preferably thienylmethyl). By heterocycle is meant in the present application in particular the piperidinyl, piperazinyl, homopiperazinyl, tetrahydrofuranyl, tetrahydropyrannyl and thiazolidinyl radicals.

By pharmaceutically acceptable salt, is meant in particular in the present application addition salts with inorganic acids such as hydrochloride, hydrobromide, hydroiodide, sulphate, phosphate, diphosphate and nitrate or with organic acids such as acetate, maleate, fumarate, tartrate, succinate, citrate, lactate, methane sulphonate, p-toluenesulphonate, pamoate and stearate. When they can be used, the salts formed from bases such as sodium or potassium hydroxide also fall within the scope of the present invention. For other examples of pharmaceutically acceptable salts, reference can be made to “Salt selection for basic drugs”, Int. J. Pharm. (1986), 33, 201-217.

Among the analogues of mikanolide and dihydromikanolide the compounds of general formula (I) described in the Examples 1 to 52 (sometimes in the form of salts) are particularly preferred, as well as their pharmaceutically acceptable salts. Even more preferred are the compounds of general formula (I) described in Examples 2, 16, 29, 37, 41 and 50 (sometimes in the form of salts) as are their pharmaceutically acceptable salts. The compound from example 50, i.e. 8-(dimethylamino)-3,10a-dimethyl-2,6-dioxodecahydro-4,7-methenofuro[3,2-c]oxireno[f]oxacylcloundecin-9-yl 2-naphthylcarbamate, and its pharmaceutically acceptable salts (in particular its hydrochloride) are even more preferred.

Among the anticancer agents which can be used in combination with mikanolide, dihydromikanolide or their analogue, there can be mentioned in particular:

enzymatic inhibitors among which:

topoisomerase inhibitors such as camptothecin and analogues of camptothecin (in the form of analogues comprising an E lactonic ring with six members such as for example the compounds described in PCT Patent Application WO 94/11376, in the form of analogues comprising an E lactonic ring with seven members such as for example the compounds described in PCT Patent Applications WO 97/00876 and WO 99/11646 or also in the form of open tetracyclic analogues such as for example the compounds described in PCT Patent Application WO 99/33829);

prenyltransferase inhibitors (and in particular described in the following Patent Applications: PCT Applications WO 97/21701, WO 97/16443, WO 98/00409, WO 96/21456, WO 97/24378, WO 97/17321, WO 97/18813, WO 95/00497, WO 00/39130; U.S. Pat. No. 5,532,359, U.S. Pat. No. 5,523,430, U.S. Pat. No. 5,510,510 and U.S. Pat. No. 5,627,202);

transduction inhibitors of heterotrimeric G protein (in particular those described in PCT Applications WO 00/02558 and WO 00/02881);

inhibitors of Cdc25 phosphatases (especially of Cdc25C phosphatases) such as those described in the as yet unpublished French Patent Application No. 01/16889;

cyclins dependent kinase (CDK) inhibitors such as those described in the as yet unpublished PCT Patent Application PCT/FR01/04048;

glycogen synthesis kinase-3 (GSK-3) inhibitors such as those described in the as yet unpublished PCT Patent Application PCT/FR01/04048

MAP kinase inhibitors;

MAP kinase kinase inhibitors such as 2-(2-amino-3-methoxyphenyl)-4H-chromen-4-one (compound PD 98059 from the company Parke Davis, described in Patent Application PCT WO 96/22985);

protein kinase C inhibitors;

tyrosine kinase inhibitors;

telomerase inhibitors;

synthesis inhibitors of puric bases such as methotrexate;

apoptosis inducers;

alkylating agents such as cisplatin, busulphan, chlorambucil, isophosphamide or procarbazine;

intercalating agents such as doxorubicin, daunorubicin, bleomycin, epirubicin, elliptinium or mitoxantrone;

anti-metabolic agents such as 5-fluorouracyl, gemcitabine or derivatives of purines such as mercaptopurine;

differentiation agents;

cell spindle poisons such as taxol and its analogues;

angiogenesis inhibitors;

anti-hormones or antagonists of steroid receptors;

antioxidants;

antisense agents;

anti-p53 agents (gene therapy);

chemo-prevention agents; +antibiotic or anti-viral agents;

immuno-therapeutic agents;

antibodies such as heregulin.

When an analogue of camptothecin comprising an E lactonic ring with seven members is used, it is preferably chosen from the following compounds:

(5R)-5-ethyl-9,10-difluoro-5-hydroxy-4,5,13,15-tetrahydro-1H,3H-oxepino[3′,4′:6,7]indolizino[1,2-b]quinoline-3,15-dione;

(5R)-1-[9-chloro-5-ethyl-5-hydroxy-10-methyl-3,15-dioxo-4,5,13,15-tetrahydro-1H,3H-oxepino[3′,4′:6,7]indolizino[1,2-b]quinolin-12-ylmethyl]-4-methyl-hexahydropyridine;

and the pharmaceutically acceptable salts of the latter.

Among the salts of (5R)-1-[9-chloro-5-ethyl-5-hydroxy-10-methyl-3,15-dioxo-4,5,13,15-tetrahydro-1H,3H-oxepino[3′,4′:6,7]indolizino[1,2-b]quinolin-12-ylmethyl]-4-methyl-hexahydropyridine, (5R)-1-[9-chloro-5-ethyl-5-hydroxy-10-methyl-3,15-dioxo-4,5,13,15-tetrahydro-1H,3H-oxepino[3′,4′:6,7]indolizino[1,2-b]quinolin-12-ylmethyl]-4-methyl-hexahydropyridinium chloride is preferred.

Generally, it is preferred to combine with the mikanolide, dihydromikanolide or one of their analogues an anticancer agent having a different action mechanism to that of said mikanolide, dihydromikanolide or analogue.

Preferably, the anticancer agent used in combination with the mikanolide, dihydromikanolide or their analogue is chosen from enzymatic inhibitors, alkylating agents, intercalating agents, anti-metabolic agents, cell spindle poisons, antibiotics and antibodies.

More preferably, the anticancer agent used in combination with the mikanolide, dihydromikanolide or their analogue is chosen from enzymatic inhibitors and alkylating agents.

Among the enzymatic inhibitors, heterotrimeric G protein transduction inhibitors, prenyltransferase inhibitors, Cdc25 phosphatase inhibitors (especially Cdc25C phosphatases), CDK inhibitors, GSK-3 inhibitors and MAP kinase inhibitors are preferred. More preferentially, the enzymatic inhibitors are chosen from heterotrimeric G protein transduction inhibitors, prenyltransferase inhibitors, Cdc25 phosphatases inhibitors (especially Cdc25C phophatases inhibitors), CDK inhibitors and GSK-3 inhibitors. Yet more preferentially, the enzymatic inhibitors are heterotrimeric G protein transduction inhibitors and prenyltransferase inhibitors (in particular farnesyltransferase inhibitors).

Among the heterotrimeric G protein transduction inhibitors, there are preferred those which are active after one hour, for example those described in PCT Patent Application WO 00/02881 (as opposed to those which are active after 24 hours such as those described in PCT Patent Application WO 00/02558). Among the prenyltransferase inhibitors, the farnesyltransferase inhibitors are preferred, and in particular those described in PCT Patent Application WO 00/39130 such as 4-(2-bromophenyl)-1-{2-[1-((4-cyano-3-methoxy)phenylmethyl)imidazo-5-yl]-1-oxoethyl}-1,2-dihydrofluoroimidazol[1,2a][1,4]-benzodiazepine.

Even more preferentially, the anticancer agent used in combination with mikanolide, dihydromikanolide or their analogue is chosen from heterotrimeric G protein transduction inhibitors and alkylating agents.

According to a preferred aspect of the invention, when the anticancer agent used in combination with mikanolide, dihydromikanolide or their analogue is a heterotrimeric G protein transduction inhibitor, it is a compound of general formula (II)

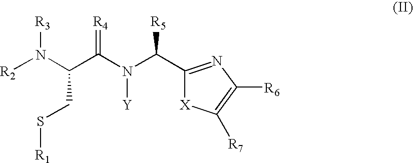

corresponding to the sub-formulae (II)₁ or (II)₂:

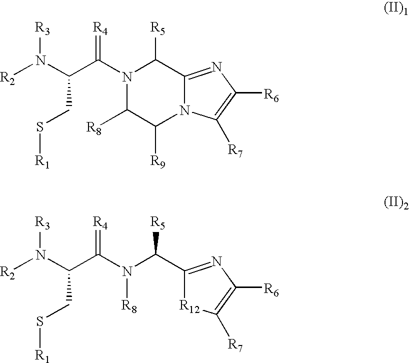

in which:

X represents R₁₂ and Y represents R₈, or X and Y complete a ring with 6 members, the X-Y assembly representing the —CH(R₈)—CH(R₉)— radical;

R₁ represents H, an alkyl or lower alkylthio radical;

R₂ and R₃ independently represent H or a lower alkyl radical;

R₄ represents H₂ or O;

R₅ represents H, or one of the following radicals: lower alkyl, lower cycloalkylalkyl, lower alkenyl, lower alkynyl, aryl, lower arylalkyl, heterocycle or lower alkyl heterocycle, these radicals can be optionally substituted by radicals chosen from the group comprising a lower alkyl, —O—R₁₀, —S(O)ₘR₁₀ (m representing 0, 1, or 2), —N(R₁₀)(R₁ I)-N—C(O)—R₁₀, —NH—(SO₂)—R₁₀, —CO₂—R₁₀, C(O)—N(R₁₀)(R₁₁), and —(SO₂)—N(R₁₀)(R₁₁) radical;

R₆ and R₇ independently represent H, a —C(O)—NH—CHR₁₃—CO₂R₁₄ radical, or one of the following radicals: lower alkyl, aryl, lower arylalkyl, heterocycle or lower alkyl heterocycle, these radicals can be optionally substituted by radicals chosen from the group comprising the OH, alkyl or lower alkoxy, N(R₁₀)(R₁₁), COOH, CON(R₁₀)(R₁₁), and halo radicals,

or R₆ and R₇ together form an aryl radical or a heterocycle;

R₈ and R₉ independently represent H, or one of the following radicals: lower alkyl, aryl, lower arylalkyl, heterocycle or lower alkyl heterocycle, these radicals can be optionally substituted by radicals chosen from the group comprising the OH, alkyl or lower alkoxy, N(R₁₀)(R₁₁), COOH, CON(R₁₀)(R₁₁) and halo radicals,

or R₈ and R₉ together form an aryl radical or a heterocycle;

R₁₀ and R₁₁ independently represent H, an aryl radical or heterocycle, or an alkyl, arylalkyl or lower alkyl heterocycle radical;

R₁₂ represents NR₉, S, or O;

R₁₃ represents a lower alkyl radical optionally substituted by a radical chosen from the lower alkyl, —OR₁₀, —S(O)ₘR₁₀ (m representing 0, 1, or 2) and —N(R₁₀)(R₁₁) radicals;

R₁₄ represents H or a lower alkyl radical;

or a pharmaceutically acceptable salt of said compound of general formula (II).

Preferably, the compound of general formula (II) corresponds to general sub-formula (II)₁. Preferably also, R₁ represents H, R₂ and R₃ independently represent H or a methyl radical, R₄ represents O, R₅ represents a lower cycloalkylalkyl, lower aryloxyalkyl, lower aralkoxyalkyl, lower arylsulphonylalkyl radical, R₆ represents an aryl radical optionally substituted by an alkyl or lower alkoxy radical (preferably methyl or methoxy) and each of R₇, R₈ and R₉ represents H.

Among the compounds of general formula (II)₁, the following compounds are particularly preferred:

7-(2-amino-1-oxo-3-thiopropyl)-8-(cyclohexylmethyl)-2-(2-methylphenyl)-5,6,7,8-tetrahydroimidazo[1,2a]pyrazine;

7-(2-amino-1-oxo-3-thiopropyl)-8-(cyclohexylmethyl)-2-phenyl-5,6,7,8-tetrahydroimidazo[1,2a]pyrazine;

7-(2-amino-1-oxo-3-thiopropyl)-2-(2-methoxyphenyl)-8-(phenylmethoxy)methyl-5,6,7,8-tetrahydroimidazo[1,2a]pyrazine;

7-(2-amino-1-oxo-3-thiopropyl)-2-(2-methoxyphenyl)-8-(1-phenylmethoxy)ethyl-5,6,7,8-tetrahydroimidazo[1,2a]pyrazine;

7-(2-amino-1-oxo-3-thiopropyl)-2-(2-methoxyphenyl)-8-(phenoxyethyl)-5,6,7,8-tetrahydroimidazo[1,2a]pyrazine;

7-(2-amino-1-oxo-3-thiopropyl)-2-(2-methoxyphenyl)-8-(phenoxyethyl)-5,6,7,8-tetrahydro-imidazo[1,2a]pyrazine, or its dimeric form;

and 7-(2-amino-1-oxo-3-thiopropyl)-2-(2-methoxyphenyl)-8-(phenylsulphonylethyl)-5,6,7,8-tetrahydro-imidazo[1,2a]pyrazine.

as well as their pharmaceutically acceptable salts.

Even more preferably, the compound of general formula (II)₁, combined with mikanolide, dihydromiknaolide or their analogue is 7-(2-amino-1-oxo-3-thiopropyl)-8-(cyclohexylmethyl)-2-phenyl-5,6,7,8-tetrahydroimidazo[1,2a]pyrazine or one of its pharmaceutically acceptable salts.

According to another preferred aspect of the invention, when the anticancer agent used in combination with mikanolide, dihydromikanolide or their analogue is a Cdc25 phosphatase inhibitor, it is a compound of general formula (III).

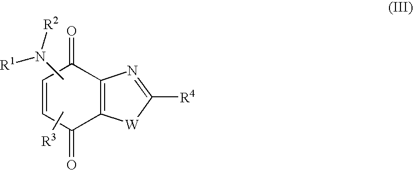

in the racemic, or enantiomeric form or in any combination of these forms, in which:

R¹ represents a hydrogen atom or a alkyl, cycloalkyl, —(CH₂)—X-Y or —(CH₂)-Z-NR⁵R⁶ radical,

R¹ can also, when W represents O, represent a carbocyclic aryl radical optionally substituted from 1 to 3 times by substituents chosen independently from a halogen atom and an alkyl, haloalkyl or alkoxy radical, X representing a bond or a linear or branched alkylene radical containing 1 to 5 carbon atoms,

Y representing a saturated carbonated cyclic system containing 1 to 3 condensed rings chosen independently from rings with 3 to 7 members, or Y representing a saturated heterocycle containing 1 to 2 heteroatoms chosen independently from O, N and S and attached to the X radical by an N or CH member, said saturated heterocycle also containing from 2 to 6 additional members chosen independently from —CHR⁷—, —CO—, —NR⁸—, —O— and —S—, R⁷ representing a hydrogen atom or an alkyl radical and R⁸ representing a hydrogen atom or an alkyl or aralkyl radical, or also Y representing a carbocyclic or heterocyclic aryl radical optionally substituted from 1 to 3 times by substituents chosen independently from the group constituted by a halogen atom, an alkyl, a haloalkyl, a alkoxy, a haloalkoxy, a hydroxy, a nitro, a cyano radical, the phenyl radical, an SO₂NHR₉ radical and an NR¹⁰R¹¹ radical, R⁹ representing a hydrogen atom or an alkyl or phenyl radical, and R₁₀ and R₁ representing independently alkyl radicals, Z representing a bond or a linear or branched alkylene radical containing 1 to 5 carbon atoms,

R⁵ and R⁶ being chosen independently from a hydrogen atom, an alkyl, aralkyl or —(CH₂)ₙ-0H radical in which n represents an integer from 1 to 6, or R⁵ and R⁶ forming together with the nitrogen atom a heterocycle with 4 to 7 members comprising 1 to 2 heteroatoms, the members necessary to complete the heterocycle being chosen independently from the —CR¹²R³—, —O—, —S— and —NR₁₄— radicals, R₁² and R₁³ representing independently each time that they occur a hydrogen atom or an alkyl radical, and R¹⁴ representing a hydrogen atom or an alkyl or aralkyl radical, or also R¹⁴ representing a phenyl radical optionally substituted from 1 to 3 times by substituents chosen independently from a halogen atom and an alkyl or alkoxy radical,

R² representing a hydrogen atom or an alkyl radical;

or also R¹ and R² forming together with the nitrogen atom a heterocycle with 4 to 7 members containing 1 to 2 heteroatoms, the members necessary to complete the heterocycle being chosen independently from the —CR¹⁵R¹⁶—, —O—, —S— and —NR¹⁷— radicals, R₁₅ and R¹⁶ representing independently each time they occur a hydrogen atom or an alkyl radical, and R¹⁷ representing a hydrogen atom or an alkyl or aralkyl radical;

R³ represents a hydrogen atom, a halogen atom, or an alkyl, haloalkyl or alkoxy radical;

R⁴ represents an alkyl, cycloalkyl, cycloalkylalkyl, cyano, amino, —CH₂—COOR¹⁸, —CH₂—CO—NR¹⁹R²⁰ or —CH₂—NR₂₁R₂₂ radical, or also R⁴ represents a heterocyclic aryl radical optionally substituted from 1 to 3 times by substituents chosen independently from a halogen atom and an alkyl, haloalkyl or alkoxy radical,

R¹⁸ representing a hydrogen atom or an alkyl radical,

R₁₉ representing a hydrogen atom, an alkyl radical or an aralkyl radical the aryl group of which is optionally substituted from 1 to 3 times by substituents chosen independently from the group constituted by a halogen atom, an alkyl, a haloalkyl, an alkoxy, a haloalkoxy, a hydroxy, a nitro, a cyano radical, the phenyl radical, an SO₂NHR²³ radical and an NR 24R₂₅ radical, R₂₃ representing a hydrogen atom or an alkyl or phenyl radical, and R²⁴ and R²⁵ representing independently alkyl radicals,

R₂₀ representing a hydrogen atom or an alkyl radical,

or also R¹⁹ and R²⁰ forming together with the nitrogen atom a heterocycle with 4 to 7 members containing from 1 to 2 heteroatoms, the members necessary to complete the heterocycle being chosen independently from the —CR²⁶R²⁷—, —O—, —S— and —NR²⁸— radicals, R²⁶ and R²⁷ representing independently each time that they occur a hydrogen atom or an alkyl radical, and R²⁸ representing a hydrogen atom or an alkyl or aralkyl radical, or also R²⁸ representing a phenyl radical optionally substituted from 1 to 3 times by substituents chosen independently from a halogen atom and an alkyl or alkoxy radical,

R²¹ representing a hydrogen atom, an alkyl radical or an aralkyl radical of which the aryl group is optionally substituted from 1 to 3 times by substituents chosen independently from the group constituted by a halogen atom, an alkyl, a haloalkyl, a alkoxy, a haloalkoxy, a hydroxy, a nitro, a cyano radical, the phenyl radical, a radical SO₂NHR²⁹ and a radical NR³⁰R³¹, R²⁹ representing a hydrogen atom or an alkyl or phenyl radical, and R³⁰ and R³¹ representing independently alkyl radicals,

R²² representing a hydrogen atom or an alkyl radical,

or also R²¹ and R²² forming together with the nitrogen atom a heterocycle with 4 to 7 members comprising 1 to 2 heteroatoms, the members necessary to complete the heterocycle being chosen independently from the —CR³²R³³—, —O—, —S— and —NR³⁴— radicals, R³² and R³³ representing independently each time that they occur a hydrogen atom or an alkyl radical, and R³⁴ representing a hydrogen atom, an alkyl radical or aralkyl radical, or also R³⁴ representing a phenyl radical optionally substituted from 1 to 3 times by substituents chosen independently from a halogen atom and an alkyl radical or an alkoxy radical; and

W represents O or S;

or a pharmaceutically acceptable salt of a compound of general formula (III) defined above.

Preferably, the compounds of general formula (III) used in combination with mikanolide, dihydromikanolide or their analogue include at least one of the following characteristics:

R₁ representing an alkyl, cycloalkyl, —(CH₂)—X-Y or —(CH₂)-Z-NR⁵R⁶ radical;

R representing a hydrogen atom or the methyl or ethyl radical;

R³ representing a hydrogen atom, a halogen atom or an alkoxy radical;

R⁴ representing an alkyl, —CH₂—COOR¹⁸ or —CH₂—CO—NR¹⁹R²⁰ or —CH₂—NR²¹R²² radical.

Generally, for a use according to the invention, the compounds of general formula (III) in which W represents a sulphur atom are preferred. Another interesting alternative for a use according to the invention consists in using the compounds of general formula (III) in which W represents an oxygen atom.

Moreover, the compounds of general formula (III) are preferably such that the X radical preferably represents a bond or a linear alkylene radical containing 1 to 5 carbon atoms. Preferably also, the compounds of general formula (III) are such that the Y radical represents a saturated carbonated cyclic system containing 1 to 3 condensed rings chosen independently from of the rings with 3 to 7 members, or Y represents a carbocyclic aryl radical (preferably optionally substituted by 1 to 3 radicals chosen from a halogen atom and an alkyl, haloalkyl, alkoxy, haloalkoxy, SO₂NHR⁹ or NR¹⁰R¹¹ radical, and more preferentially optionally substituted by 1 to 3 radicals chosen from a halogen atom and an alkyl, alkoxy, SO₂NHR⁹ or NR¹⁰R¹¹ radical) or also Y represents a heterocyclic aryl radical, said heterocyclic aryl radical being preferably chosen from the aryl radicals with 5 members (and in particular from the imidazolyl and thienyl radicals) and preferably optionally substituted by 1 to 3 radicals chosen from a halogen atom and an alkyl, haloalkyl, alkoxy, haloalkoxy, SO₂NHR⁹ or NR¹⁰R¹¹ radical, and more preferentially optionally substituted by 1 to 3 radicals chosen from a halogen atom and an alkyl, alkoxy, SO₂NHR⁹ or NR¹⁰R₁₁ radical; R⁹ preferably represents a hydrogen atom. In addition, the compounds of general formula (III) are preferably such that the Z radical represents an alkylene radical containing 1 to 5 carbon atoms. Preferably also, the compounds of general formula (III) are such that R₅ and R₆ are chosen independently from a hydrogen atom and an alkyl radical, or also R₅ and R₆ form together with the nitrogen atom which carries them a heterocycle with 4 to 7 members comprising 1 to 2 heteroatoms, said heterocycle being then preferably one of the azetidinyl, pyrrolidinyl, piperidinyl, piperazinyl, homopiperazinyl, morpholinyl and thiomorpholinyl radicals optionally substituted by 1 to 3 alkyl radicals (and preferably by 1 to 3 methyl radicals). The compounds of general formula (III) are preferably also such that R¹⁸ represents a hydrogen atom or the methyl or ethyl radical.

In addition, the compounds of general formula (III) are such that the R⁷, R¹², R³, R¹⁵, R¹⁶, R²⁶, R²⁷, R³², R³³ and R³⁴ radicals are preferably chosen independently from a hydrogen atom and a methyl radical and the R⁸, R⁴, R⁷, R²⁸ and R³⁴ radicals are preferably chosen independently from a hydrogen atom and a methyl or benzyl radical.

Moreover, as regards R¹⁹ and R²⁰ in the compounds of general formula (III), the cases in which R¹⁹ represents a hydrogen atom, an alkyl radical or a benzyl radical and R²⁰ represents a hydrogen atom or the methyl radical, as well as those in which R¹⁹ and R²⁰ form together with the nitrogen atom which carries them a heterocycle with 4 to 7 members comprising 1 to 2 heteroatoms, said heterocycle being then preferably one of the radicals azetidinyl, pyrrolidinyl, piperidinyl, piperazinyl, homopiperazinyl, morpholinyl and thiomorpholinyl optionally substituted by 1 to 3 alkyl radicals (and preferably optionally substituted by 1 to 3 methyl radicals).

Finally, as regards R²¹ and R²² in the compounds of general formula (III), the cases in which R²¹ represents a hydrogen atom, an alkyl radical or a benzyl radical and R²² represents a hydrogen atom or the radical methyl, as well as those in which R²¹ and R²² form together with the nitrogen atom which carries them a heterocycle with 4 to 7 members comprising 1 to 2 heteroatoms, said heterocycle being then preferably one of the radicals azetidinyl, pyrrolidinyl, piperidinyl, piperazinyl, homopiperazinyl, morpholinyl and thiomorpholinyl optionally substituted by 1 to 3 alkyl radicals (and preferably optionally substituted by 1 to 3 methyl radicals).

More preferentially, the compounds of general formula (III) used in combination with mikanolide, dihydromikanolide or their analogue, the invention include at least one of the following characteristics:

R¹ representing an alkyl, cycloalkyl, (cycloalkyl)alkyl or —(CH₂)-Z-NR₅R₆ radical;

R² representing a hydrogen atom or the methyl radical;

R³ representing a hydrogen atom, a halogen atom or the methoxy radical;

R⁴ representing an alkyl radical or —CH₂—NR²¹R²².

Even more preferentially, the compounds of general formula (III) used in combination with mikanolide, dihydromikanolide or their analogue include at least one of the following characteristics:

R¹ representing a —(CH₂)-Z-NR⁵R⁶ radical;

R² representing a hydrogen atom;

R³ representing a hydrogen atom or a halogen atom (said halogen atom preferably being a chlorine atom);

R⁴ representing an alkyl radical, and preferably an alkyl radical containing 1 to 4 carbon atoms, and more preferentially still a methyl or ethyl radical.

According to a particular variant of the invention, the compounds of general formula (III) used in combination with mikanolide, dihydromikanolide or their analogue are such that W represents O. In this particular case, it is preferred that R¹ represents an aryl radical, and in particular a phenyl radical, optionally substituted from 1 to 3 times by substituents chosen independently from a halogen atom and an alkyl, haloalkyl or alkoxy radical. More preferentially, and still when the compounds of general formula (III) used in combination with mikanolide, dihydromikanolide or their analogue are such that W represents O, it is preferred that R¹ represents a phenyl radical optionally substituted by a halogen atom (said halogen atom preferably being a fluorine atom).

Compounds of general formula (III) preferred for a use in combinations according to the present invention are the compounds described in Examples 53 to 69 hereafter, as well as their pharmaceutically acceptable salts. Quite particularly, when it is chosen to combine a compound of general formula (III) with mikanolide, dihydromikanolide or their analogue 5-{[2-(dimethylamino)ethyl]amino}-2-methyl-1,3-benzothiazole-4,7-dione or one of its pharmaceutically acceptable salts is preferably used.

According to yet another preferred aspect of the invention, when the anti-cancer agent used in combination with mikanolide, dihydromikanolide or their analogue is a CDK and/or GSK-3 inhibitor, this will be a compound of general formula (IV)

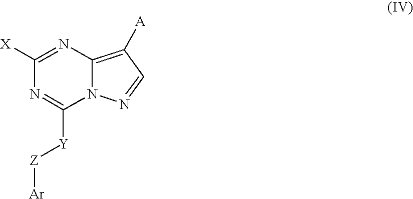

in racemic, or enantiomeric form or in any combination of these forms, in which

A represents a hydrogen atom, a halogen atom, a formyl, cyano, nitro, guanidinoaminomethylenyl, (1,3-dihydro-2-oxoindol)-3-ylidenemethyl, alkylcarbonyl, aralkylcarbonyl or heteroaralkylcarbonyl radical, or also an -L-NR₁R² radical in which L represents an alkylene radical and R₁ and R² are chosen independently from a hydrogen atom and an alkyl radical or R₁ and R₂ taken together with the nitrogen atom which carries them form a heterocycle with 5 to 7 members, the complementary members being chosen independently from the group comprising —CH₂—, —NR₃—, —S— and —O—, R³ representing independently each time that it occurs a hydrogen atom or an alkyl radical;

X represents a hydrogen atom, an alkylthio, aralkylthio, alkylthioxo or aralkylthioxo radical, or also an NR⁴R⁵ radical in which R⁴ represents an alkyl radical, a hydroxyalkyl radical, a cycloalkyl radical optionally substituted by one or more radicals chosen from the alkyl, hydroxy and amino radicals, an aralkyl radical the aryl radical of which is optionally substituted by one or more radicals chosen from a halogen atom, the cyano radical, the nitro radical and the alkyl or alkoxy radicals, or also R⁴ represents a heteroaryl or heteroarylalkyl radical, the heteroaryl radical of the heteroaryl or heteroarylalkyl radicals being optionally substituted by one or more alkyl radicals and R⁵ represents a hydrogen atom, or also R⁴ and R⁵ taken together with the nitrogen atom which carries them form a heterocycle with 5 to 7 members, the complementary members being chosen independently from the group comprising —CH₂—, —NR⁶—, —S— and —O—, R⁶ representing independently each time that it occurs a hydrogen atom or an alkyl or hydroxyalkyl radical;

Y represents NH or an oxygen atom;

Z represents a bond or an alkyl or alkylthioalkyl radical; and

Ar represents a carbocyclic aryl radical optionally substituted from 1 to 3 times by radicals chosen independently from a halogen atom, the cyano radical, the nitro radical, an alkyl or alkoxy radical and an NR⁷R⁸ radical in which R⁷ and R⁸ independently represent a hydrogen atom or an alkyl radical or R⁷ and R⁸ taken together with the nitrogen atom which carries them form a heterocycle with 5 to 7 members, the complementary members being chosen independently from the group comprising —CH₂—, —NR⁹—, —S— and —O—, R⁹ representing independently each time that it occurs a hydrogen atom or an alkyl radical,

or also Ar represents a heterocyclic aryl radical containing 5 or 6 members and in which the heteroatom or the heteroatoms are chosen from nitrogen, oxygen or sulphur atoms, said heteroatoms can be optionally oxidized (Ar can represent for example the oxidopyridyl radical) and said heterocyclic aryl radical can be optionally substituted by one or more radicals chosen independently from the alkyl, aminoalkyl, alkylaminoalkyl and dialkylaminoalkyl radicals;

or a pharmaceutically acceptable salt of a compound of general formula (IV) defined above.

Preferably, the compounds of general formula (IV) are such that they present at least one of the following characteristics:

A represents a hydrogen atom, a halogen atom, a formyl, cyano, nitro, guanidinoaminomethylenyl, (1,3-dihydro-2-oxoindol)-3-ylidenemethyl, alkylcarbonyl or aralkylcarbonyl radical, or also an -L-NR¹R² radical in which L represents an alkylene radical and R¹ and R² are chosen independently from a hydrogen atom and an alkyl radical or R¹ and R² taken together with the nitrogen atom which carries them form a heterocycle with 5 to 7 members, the complementary members being chosen independently from the group comprising —CH₂—, —NR₃—, —S— and —O—, R³ representing independently each time that it occurs a hydrogen atom or an alkyl radical;

X represents a hydrogen atom, an alkylthio or alkylthioxo radical, or also an NR⁴R⁵ radical in which R⁴ represents an alkyl radical, a hydroxyalkyl radical, a cycloalkyl radical optionally substituted by one or more amino radicals, an aralkyl radical the aryl radical of which is optionally substituted by one or more radicals chosen from a halogen atom and the alkyl or alkoxy radicals, or also R⁴ represents a heteroaryl or heteroarylalkyl radical, the heteroaryl radical of the heteroaryl or heteroarylalkyl radicals being optionally substituted by one or more alkyl radicals and R⁵ represents a hydrogen atom, or also R⁴ and R⁵ taken together with the nitrogen atom which carries them form a heterocycle with 5 to 7 members, the complementary members being chosen independently from the group comprising —CH₂—, —NR⁶—, —S— and —O—, R⁶ representing independently each time that it occurs a hydrogen atom or an alkyl or hydroxyalkyl radical.

More preferentially, the compounds of general formula (IV) are such that they present at least one of the following characteristics:

A represents a halogen atom, a formyl, guanidinoaminomethylenyl, (1,3-dihydro-2-oxoindol)-3-ylidenemethyl or alkylcarbonyl radical, or also an -L-NR₁R² radical in which L represents a methylene radical and R₁ and R² are chosen independently from a hydrogen atom and an alkyl radical or R₁ and R² taken together with the nitrogen atom which carries them form a heterocycle with 5 to 7 members, the complementary members being chosen independently from the group comprising —CH₂—, —NR³— and —O—, R³ representing independently each time that it occurs a hydrogen atom or an alkyl radical;

X represents an alkylthio or alkylthioxo radical, or also an NR⁴R⁵ radical in which R⁴ represents an alkyl radical, a hydroxyalkyl radical, a cycloalkyl radical optionally substituted by one or more amino radicals, or also R⁴ represents a heteroaryl or heteroarylalkyl radical, the heteroaryl radical of the heteroaryl or heteroarylalkyl radicals being optionally substituted by one or more alkyl radicals and R⁵ represents a hydrogen atom, or also R⁴ and R⁵ taken together with the nitrogen atom which carries them form a heterocycle with 5 to 7 members, the complementary members being chosen independently from the group comprising —CH₂—, —NR₆— and —O—, R⁶ representing independently each time that it occurs a hydrogen atom or an alkyl or hydroxyalkyl radical;

Z represents a bond or an alkyl radical;

Ar represents a carbocyclic aryl radical optionally substituted from 1 to 3 times by radicals chosen independently from a halogen atom and an NR⁷R⁸ radical in which R⁷ and R⁸ independently represent a hydrogen atom or an alkyl radical or R⁷ and R⁸ taken together with the nitrogen atom which carries them form a heterocycle with 5 to 7 members, the complementary members being chosen independently from the group comprising —CH₂—, —NR⁹— and —O—, R₉ representing independently each time that it occurs a hydrogen atom or an alkyl radical,

or also Ar represents a heterocyclic aryl radical containing 5 or 6 members and the heteroatom or the heteroatoms of which are chosen from nitrogen and oxygen atoms, said heteroatoms can be optionally oxidized and said heterocyclic aryl radical can be optionally substituted by one or more radicals chosen independently from the alkyl, aminoalkyl, alkylaminoalkyl and dialkylaminoalkyl radicals.

Also more preferentially, the compounds of general formula (IV) are such that they present at least one of the following characteristics:

A represents a halogen atom, a formyl, guanidinoaminomethylenyl, (1,3-dihydro-2-oxoindol)-3-ylidenemethyl or alkylcarbonyl radical, or also a radical -L-NR¹R² in which L represents a methylene radical and R₁ and R² are chosen independently from a hydrogen atom and an alkyl radical or R₁ and R² taken form together with the nitrogen atom which carries them a heterocycle with 5 to 7 members, the complementary members being chosen independently from the group comprising —CH₂—, —NR³— and —O—, R³ representing independently each time that it occurs a hydrogen atom or an alkyl radical;

X represents an alkylthio radical (and preferably methylthio) or alkylthioxo (and preferably methylthioxo), or also an NR⁴R⁵ radical in which R⁴ represents an alkyl radical, a hydroxyalkyl radical, a cycloalkyl radical (and preferably cyclohexyl) optionally substituted by one or more amino radicals, or also R₄ represents a heteroaryl or heteroarylalkyl radical, the heteroaryl radical of the heteroaryl or heteroarylalkyl radicals being optionally substituted by one or more alkyl radicals and R⁵ represents a hydrogen atom, or also R⁴ and R⁵ taken together with the nitrogen atom which carries them form a heterocycle with 5 to 7 members, the complementary members being chosen independently from the group comprising —CH₂— and —NR⁶—, R⁶ representing independently each time that it occurs a hydrogen atom or an alkyl or hydroxyalkyl radical;

Y represents NH;

Z represents a bond or a —CH₂— radical;

Ar represents a carbocyclic aryl radical (said carbocyclic aryl radical preferably being a phenyl radical) optionally substituted from 1 to 3 times by radicals chosen independently from a halogen atom and an NR⁷R⁸ radical in which R⁷ and R⁸ independently represent a hydrogen atom or an alkyl radical or R⁷ and R⁸ taken together with the nitrogen atom which carries them form a heterocycle with 5 to 7 members, the complementary members being chosen independently from the group comprising —CH₂— and —NR⁹—, R⁹ representing independently each time that it occurs an alkyl radical,

or also Ar represents a heterocyclic aryl radical with 5 or 6 members and in which the heteroatom or the heteroatoms are chosen from nitrogen and oxygen atoms (said heterocyclic aryl radical preferably being a pyridyl radical), said heteroatoms can optionally be oxidized and said heterocyclic aryl radical can be optionally substituted by one or more radicals chosen independently from the alkyl, aminoalkyl, alkylaminoalkyl and dialkylaminoalkyl radicals.

Compounds of general formula (IV) preferred for a use in combinations according to the present invention are the compounds described (sometimes in the form of salts) in Examples 70 to 102 hereafter, as well as their pharmaceutically acceptable salts.

A compound of general formula (IV) particularly preferred for obtaining a product according to the invention is 8-bromo-2-(1R-isopropyl-2-hydroxyethylamino)-4-(3-fluorophenylmethylamino)-pyrazolo[1,5-a]-1,3,5-triazine, 8-bromo-2-(1R-isopropyl-2-hydroxyethylamino)-4-(3-pyridylmethylamino)pyrazolo[1,5-a]-1,3,5-triazine or a pharmaceutically acceptable salt of the latter compounds. Even more particularly it is preferred to use 8-bromo-2-(1R-isopropyl-2-hydroxyethylamino)₄-(3-pyridylmethylamino)pyrazolo[1,5-a]-1,3,5-triazine or one of its pharmaceutically acceptable salts.

Also according to a preferred aspect of the invention, when the anti-cancer agent used in combination with mikanolide, dihydromikanolide or their analogue is a farnesyltransferase inhibitor, this is a compound of general formula (V)

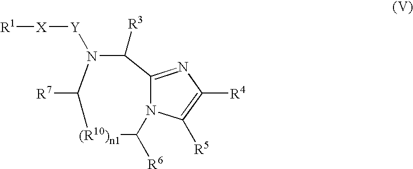

in which:

n1 represents 0 or 1;

X represents, independently each time that it occurs, (CHR¹¹)ₙ₃(CH₂)ₙ₄Z(CH₂)ₙ₅;

Z representing O, N(R¹²), S, or a bond;

n3 representing, independently each time that it occurs, 0 or 1;

each of n4 and n5 representing, independently at each time that they occur, 0, 1, 2, or 3;

Y represents, independently each time that it occurs, CO, CH₂, CS, or a bond;

R₁ represents one of the radicals

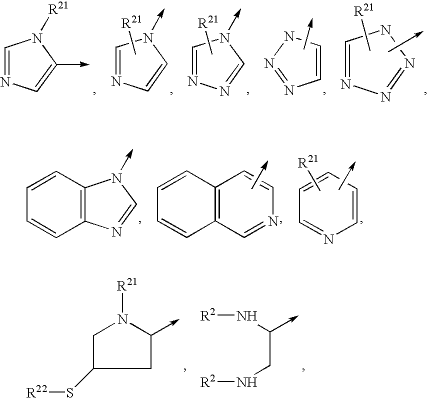

or N(R²⁴R²⁵); each of R², R₁, and R¹² representing, independently each time that it occurs, H or an optionally substituted radical chosen from the group consisting of a (C₁₋₆)alkyl radical and an aryl radical, said optionally substituted radical being optionally substituted by at least one radical chosen from the R⁸ and R³⁰ radicals, each substituent being chosen independently of the others;

R³ represents, independently each time that it occurs, H or an optionally substituted radical chosen from the group consisting of the (C₁₋₆)alkyl, (C₂₋₆)alkenyl, (C₂₋₆)alkynyl, (C₃₋₆)cycloalkyl, (C₃₋₆)cycloalkyl(C₁₋₆)alkyl, (C₅₋₇)cycloalkenyl, (C₅₋₇)cycloalkenyl(C₁₋₆)alkyl, aryl, aryl(C₁₋₆)alkyl, heterocyclyl, and heterocyclyl(C₁₋₆)alkyl radicals, said optionally substituted radical being optionally substituted by at least one radical chosen from the R³⁰ radicals, each substituent being chosen independently of the others;

each of R⁴ and R⁵ represent, independently each time that it occurs, H or an optionally substituted radical chosen from the group consisting of the (C₁₋₆)alkyl, (C₃₋₆)cycloalkyl, aryl and heterocyclyl radicals, said optionally substituted radical being optionally substituted by at least one radical chosen from the R³⁰ radicals, each substituent being chosen independently of the others, or R⁴ and R⁵ taken together with the carbon atoms to which they are attached together form an aryl radical;

R⁶ represents, independently each time that it occurs, H or an optionally substituted radical chosen from the group consisting of the (C₁₋₆)alkyl, (C₂₋₆)alkenyl, (C₃₋₆)cycloalkyl, (C₃₋₆)cycloalkyl(C₁₋₆)alkyl, (C₅₋₇)cycloalkenyl, (C₅₋₇)cycloalkenyl(C₁₋₆)alkyl, aryl, aryl(C₁₋₆)alkyl, heterocyclyl and heterocyclyl(C₁₋₆)alkyl radicals, said optionally substituted radical being optionally substituted by at least one radical chosen from the OH, (C₁₋₆)alkyl, (C₁₋₆)alkoxy, —N(R⁸R⁹), —COOH, —CON(R⁸R⁹) and halo radicals, each substituent being chosen independently of the others;

R⁷ represents, independently each time that it occurs, H, ═O, ═S, H or an optionally substituted radical chosen from the group consisting of the (C₁₋₆)alkyl, (C₂₋₆)alkenyl, (C₃₋₆)cycloalkyl, (C₃₋₆)cycloalkyl(C₁₋₆)alkyl, (C₅₋₇)cycloalkenyl, (C₅₋₇)cycloalkenyl(C₁₋₆)alkyl, aryl, aryl(C₁₋₆)alkyl, heterocyclyl and heterocyclyl(C₁₋₆)alkyl radicals, said optionally substituted radical being optionally substituted by at least one radical chosen from the OH, (C₁₋₆)alkyl, (C₁₋₆)alkoxy, —N(R⁸R⁹), —COOH, —CON(R⁸R⁹) and halo radicals, each substituent being chosen independently of the others;

each of R₈ and R⁹ representing, independently each time that it occurs, H, (C₁₋₆)alkyl, (C₂₋₆)alkenyl, (C₂₋₆)alkynyl, aryl, or aryl(C₁₋₆)alkyl;

R₁₀ represents C;

or, when n1=0, R⁶ and R⁷ can be taken together with the carbon atoms to which they are attached to form a aryl or cyclohexyl radical;

R²¹ represents, independently each time that it occurs, H or an optionally substituted radical chosen from the group consisting of the (C₁₋₆)alkyl and aryl(C₁₋₆)alkyl radicals, said optionally substituted radical being optionally substituted by at least one radical chosen from the R⁸ and R³⁰ radicals, each substituent being chosen independently of the others;

R²² represents H, (C₁₋₆)alkylthio, (C₃₋₆)cycloalkylthio, R⁸—CO—, or a substituent of Formula

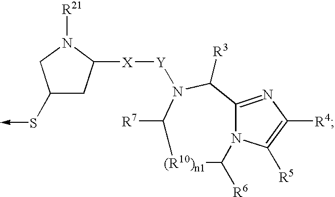

each of R²⁴ and R²⁵ represents, independently each time that it occurs, H, (C₁₋₆)alkyl or aryl(C₁₋₆)alkyl;

R³⁰ represents, independently each time that it occurs, (C₁₋₆)alkyl, —O—R⁸, —S(O)ₙ₆R⁸, —S(O)ₙ₇N(R₈R⁹), —N(R⁸R⁹), —CN, —NO₂, —CO₂R⁸, —CON(R⁸R⁹), —NCO—R⁸, or halogen, each of n6 and n7 representing, independently each time that it occurs, 0, 1 or 2;

said radical heterocyclyl being azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulphone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, 2-oxopiperazinyl, 2-oxopiperidinyl, 2-oxopyrrolidinyl, piperidyl, piperazinyl, pyridyl, pyridyl-N-oxide, quinoxalinyl, tetrahydrofuryl, tetrahydroisoquinolinyl, tetrahydro-quinolinyl, thiamorpholinyl, thiamorpholinyl sulphoxide, thiazolyl, thiazolinyl, thienofuryl, thienothienyl or thienyl;

said aryl radical being phenyl or naphthyl;

it being understood that:

when n1=1, R₁₀ is C and R⁶ represents H, then R¹⁰ and R⁷ can form, taken together, the radical

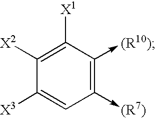

or when n1=1, R₁₀ is C, and R⁷ is =O, —H, or ═S, then R₁₀ and R⁶ can form, taken together, the radical

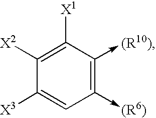

with each of X¹, X², and X³ representing, independently, H, a halogen atom, —NO₂, —NCO—R⁸, —CO₂R⁸, —CN, or —CON(R⁸R⁹); and

when R₁ is N(R²⁴R²⁵), then n3 represents 1, each of n4 and n5 represents 0, Z is a bond, and R³ and R¹¹ can form, taken together, the radical

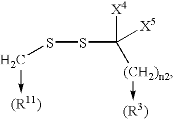

with n2 representing an integer from 1 to 6, and each of X⁴ and X⁵ representing, independently, H, (C₁₋₆)alkyl or aryl, or X⁴ and X⁵ forming, taken together, a (C₃₋₆)cycloalkyl radical;

or a pharmaceutically acceptable salt of a compound of general formula (V) defined above.

Preferably, when they are used for the invention, the compounds of general formula (V) are those in which are found, independently, the radicals presenting the following characteristics:

R¹ representing the radical

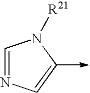

R²¹ representing an aralkyl radical the aryl group of which can be optionally substituted by one or more radicals chosen from a halogen atom and the cyano, hydroxy, alkoxy, amino, alkylamino and dialkylamino radicals;

R⁴ representing an aryl radical optionally substituted by one or more radicals chosen from a halogen atom and the hydroxy, alkoxy, amino, alkylamino and dialkylamino radicals;

X representing an alkylene radical containing from 1 to 6 carbon atoms;

Y representing CO;

n1=1, R₁₀ being C, R⁶ representing H and R¹⁰ and R⁷ forming, taken together, the radical

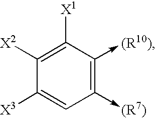

each of X¹, X², and X³ representing, independently, H or a halogen atom.

Compounds of general formula (V) particularly preferred for a use in combinations according to the present invention are 1-(2-(1-((4-cyano)phenylmethyl)imidazol-4-yl)-1-oxoethyl-2,5-dihydro-4-(2-methoxyphenyl)imidazo[1,2c][1,4]benzodiazepine and 4-(2-bromophenyl)-1-(2-(1-((4-cyano-3-methoxy)phenylmethyl)-imidazo-5-yl)-1-oxoethyl)-1,2-dihydro-8-fluoro-imidazo[1,2a][1,4]-benzodiazepine, as well as their pharmaceutically acceptable salts (and still more preferentially 4-(2-bromophenyl)-1-(2-(1-((4-cyano-3-methoxy)phenylmethyl)-imidazo-5-yl)-1-oxoethyl)-1,2-dihydro-8-fluoro-imidazo[1,2a][1,4]-benzodiazepine and its pharmaceutically acceptable salts).

According to a particular aspect of the invention, the anti-cancerous agent used in combination with mikanolide, dihydromikanolide or their analogue is preferably chosen from 7-(2-amino-1-oxo-3-thiopropyl)-8-(cyclohexylmethyl)-2-phenyl-5,6,7,8-tetrahydroimidazo[1,2a]pyrazine, cisplatin, 8-bromo-2-(1R-isopropyl-2-hydroxyethylamino)-4-(3-pyridylmethylamino)pyrazolo[1,5-a]-1,3,5-triazine, 8-bromo-2-(1R-isopropyl-2-hydroxyethylamino)-4-(3-fluorophenylmethylamino)-pyrazolo[1,5-a]-1,3,5-triazine, 1-(2-(1-((4-cyano)phenylmethyl)imidazol-4-yl)-1-oxoethyl-2,5-dihydro-4-(2-methoxyphenyl)imidazo[1,2c][1,4]benzodiazepine, 4-(2-bromophenyl)-1-(2-(1-((4-cyano-3-methoxy)phenylmethyl)-imidazo-5-yl)-1-oxoethyl)-1,2-dihydro-8-fluoro-imidazo[1,2a][1,4]-benzodiazepine and their pharmaceutically acceptable salts. Also more particularly, the anti-cancerous agent used in combination with mikanolide, dihydromikanolide or their analogue is chosen from 7-(2-amino-1-oxo-3-thiopropyl)-8-(cyclohexylmethyl)-2-phenyl-5,6,7,8-tetrahydroimidazo[1,2a]pyrazine and cisplatin.

Preferably also, the cancer treated with the product according to the invention is chosen from cancers of the oesophagus, stomach, intestines, rectum, oral cavity, pharynx, larynx, lung, colon, breast, cervix uteri, corpus endometrium, ovaries, prostate, testicles, bladder, kidneys, liver, pancreas, bones, connective tissue, skin, for example melanomas, eyes, brain and central nervous system, as well as cancer of the thyroid, leukemia, Hodgkin's disease, lymphomas other than those related to Hodgkin's and multiple myelomas.

More preferentially, the cancers treated by the product according to the invention are cancers of the digestive system, and in particular cancers of the oral cavity, oesophagus, stomach, intestines, colon or rectum.

A subject of the invention is also a pharmaceutical composition comprising at least one of the products according to the invention, in other words a composition containing, as an active ingredient, the combination of mikanolide, dihydromikanolide or their analogue with another anti cancer agent.

The preferences indicated for the products of the invention are applicable mutatis mutandis to the pharmaceutical compositions according to the invention.

The pharmaceutical compositions comprising a product according to the invention can be in the form of solids, for example powders, granules, tablets, gelatin capsules, liposomes or suppositories. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax.

The pharmaceutical compositions comprising a product according to the invention can also be presented in liquid form, for example solutions, emulsions, suspensions or syrups. Appropriate liquid supports can be, for example, water, organic solvents such as glycerol or glycols, as well as their mixtures, in varying proportions, in water.

The administration of a medicament according to the invention can be carried out by topical, oral, parenteral route, by injection (intramuscular, sub-cutaneous, intravenous, intraperitoneal, etc.), etc. The administration route of course depends on the type of disease to be treated.

The dose of a product according to the present invention envisaged for the treatment of the diseases or disorders mentioned above, varies according to the administration method, the age and body weight of the subject to be treated as well as the latter's condition, and the final decision is made by the attending doctor or veterinary surgeon. Such a quantity determined by the attending doctor or veterinary surgeon is here referred to as “effective therapeutic quantity”.

For information, the following administration doses (daily, unless specified otherwise) can be envisaged for the different compounds forming part of the composition of a product according to the invention:

mikanolide, dihydromikanolide or their analogue corresponding to general formula (I): from 1 to 100 mg/kg by intraperitoneal route;

compound of general formula (II): from 50 to 200 mg/m² by intraperitoneal route;

cisplatin: from 50 to 80 mg/m²;

taxol: from 1 to 20 mg/kg (intraperitoneal route) or 1 to 3 mg/kg (intravenous route).

The compounds forming part of the composition of the product of the invention can be prepared by the processes described hereafter.

Preparation of the Products of the Invention:

Preparation of the Compounds of General Formula (I)

The preparation of the compounds of general formula (I) is described hereafter (this is the relevant extract from unpublished PCT Patent Application no. PCT/FR02/00092).

Preparation of the Compounds of General Sub-Formula (I)₁:

Case 1: R₁=—H:

The preparation of this type of compounds is summarized in Diagram 1 hereafter.

Dihydromikanolide by adding a nucleophile such as a primary or secondary amine HNR₆R₇, or also a thiol R₆SH in the presence of a base, in an inert solvent such as tetrahydrofuran or acetone, at a temperature preferably comprised between 0° C. and 50° C., and more preferentially at ambient temperature.

In the case where R₃ is not OH, the intermediate obtained is treated with one of the reagents of general formula R₁₄(CO)-Hal (or an equivalent reagent such as for example the anhydride (R₁₄(CO))₂O), R₁₈O(CO)-Hal, Hal-Si R₁₅R₁₆R₁₇ (Hal representing a halogen atom) or R₁₈—NCO in order to obtain the desired final compound. In general, this reaction is carried out in an aprotic solvent such as dichloromethane, trichloroethane, acetonitrile, tetrahydrofuran or toluene, at a temperature preferably comprised between 0° C. and 110° C. and optionally in the presence of a base such as triethylamine or 4-dimethylaminopyridine. These types of reaction are well known to a person skilled in the art (who can in particular usefully consult the following reference work: Greene et al., “Protective groups in Organic Synthesis”, 2nd edition, Wiley, New York, 1991) owing to their frequent use for protecting an alcohol or amine function. For example, as regards the silylation reaction, this is generally carried out by treatment of an alcoholic compound with a silyl chloride in the presence of a base, in an aprotic solvent at a temperature comprised between 0° C. and 50° C.

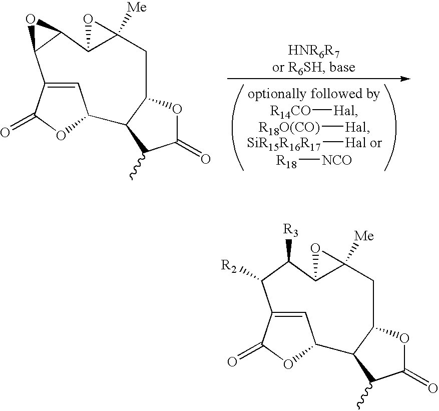

### Diagram A.1

An additional method for obtaining compounds with R₃═OCOR₁₄ consists in treating the intermediate alcohol with the R₁₄—COOH acid in the presence of a base, such as for example dimethylaminopyridine, and a coupling agent, such as for example 1-(3-dimethylaminopropyl)-3-ethylcarbodiimide hydrochloride (EDC.HCl).

Case 2: R₁=R₂≠H:

The compounds of formula (I)₁ in which R₁=R₂≠H and R₃ represents a hydroxyl group can be prepared from mikanolide by adding a nucleophile such as a primary or secondary amine HNR₄R₅, or also a thiol R₄SH in the presence of a base, in an inert solvent such as tetrahydrofuran or acetone, at a temperature preferably comprised between 0° C. and 50° C., and more preferentially at ambient temperature.

In the case where R₃ is not OH, a second reaction is carried out using a compound of general formula R₁₄(CO)-Hal (or an equivalent reagent such as for example the anhydride (R₁₄(CO))₂O), R₁₈0(CO)-Hal, Hal-SiR₁₅R₁₆R₁₇ (Hal representing a halogen atom) or R₁₈—NCO in order to obtain the desired final compound. This reaction can be carried out in a manner analogous to that described in CASE 1.

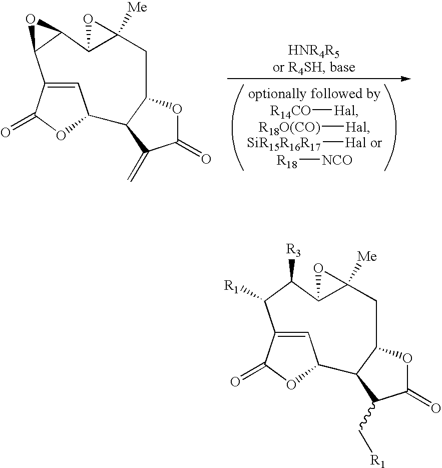

### Diagram A.2

An additional method for obtaining compounds with R₃═OCOR₁₄ consists in treating the intermediate alcohol with the acid R₁₄—COOH in the presence of a base, such as for example dimethylaminopyridine, and a coupling agent, such as for example 1-(3-dimethylaminopropyl)-3-ethylcarbodiimide hydrochloride (EDC.HCl).

Case 3: R₁≠H and R₂:

In this case, the compound of general formula (I)₂ is subjected to the same reactions as in CASE 1 in order to produce the desired final compound in which R₁: R₂.

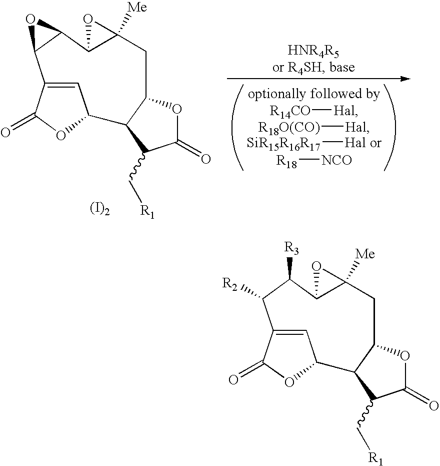

### Diagram A.3

Preparation of the Compounds of General Sub-Formula (I)₂:

The compounds of sub-formula (I)₂ can be prepared, Diagram A.4, from mikanolide by adding a nucleophile such as a primary or secondary amine HNR₆R₇, or also a thiol R₆SH in the presence of a base, in an inert solvent such as tetrahydrofuran or acetone, at a temperature preferably comprised between 0° C. and 50° C., and more preferentially at ambient temperature.

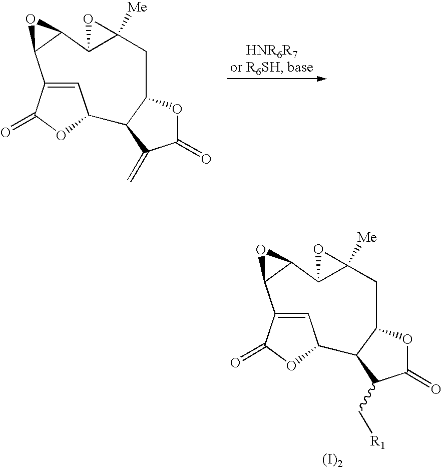

### Diagram A.4

Salts of the Compounds of General Formula (I):

Certain compounds of the invention can be prepared in the form of pharmaceutically acceptable salts according to the usual methods. As regards these salts, a person skilled in the art can usefully consult the article by Gould et al., “Salt selection for basic drugs”, Int. J. Pharm. (1986), 33, 201-217.

B. Preparation of the Compounds of General Formula (II)

For the preparation of the compounds of general formula (II), a person skilled in the art can proceed on the basis of the description provided in PCT Patent Applications WO 97/30053 and WO 00/02881.

C. Preparation of the Compounds of General Formula (III)

The preparation processes hereafter are given as an indication and a person skilled in the art can subject them to variations which he judges to be necessary, both as regards the reagents and the conditions and techniques of the reactions.

General Method

Generally, the compounds of general formula (III) can be prepared according to the procedure summarised in Diagram C.1 hereafter.

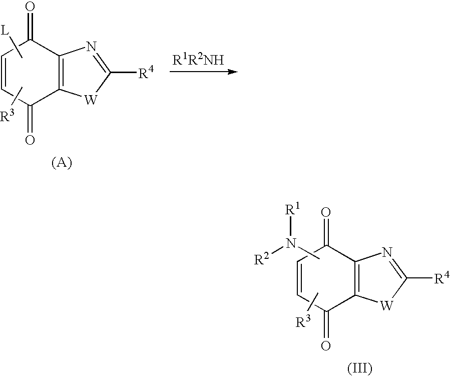

### Diagram C.1

According to this method, the compounds of general formula (III), in which R¹, R², R³, R⁴ and W are as described above, are obtained by treatment of the compounds of general formula (A), in which L represents a methoxy radical, a halogen atom or a hydrogen atom and R³, R⁴ and W have the same meaning as in general formula (III), with amines of general formula NR¹R²H in a protic solvent such as the methanol or the ethanol, at a temperature of between 0° C. and 50° C. and optionally in the presence of a base such as, for example, diisopropylethylamine (Yasuyuki Kita et al., J. Org. Chem. (1996), 61, 223-227).

In the particular case where the compounds of general formula (A) are such that L and R³ each represent a halogen atom, the compounds of general formula (III) can be obtained in the form of a mixture of the 2 position isomers, but it is then possible to separate them by chromatography on a silica column in an appropriate eluent.

Alternatively, the compounds of general formula (III) in which R³ represents a halogen atom (Hal) can be obtained, Diagram C.1a, from compounds of general formula (III) in which R³ represents a hydrogen atom, for example by the action of N-chlorosuccinimide or N-bromosuccinimide in an aprotic solvent such as dichloromethane or tetrahydrofuran (Paquette and Farley, J. Org. Chem. (1967), 32, 2725-2731), by the action of an aqueous solution of sodium hypochlorite (Javel water) in a solvent such as acetic acid (Jagadeesh et al., Synth Commun. (1998), 28, 3827-3833), by the action of Cu(II) (in a mixture CuCl₂/HgCl₂) in the presence of a catalytic quantity of iodine in a solvent such as warm acetic acid (Thapliyal, Synth. Commun. (1998), 28, 1123-1126), by the action of an agent such as benzyltrimethylammonium dichloroiodate in the presence of NaHCO₃ in a solvent such as a dichloromethane/methanol mixture (Kordik and Reitz, J. Org. Chem. (1996), 61, 5644-5645), or also by the use of chlorine, bromine or iodine in a solvent such as dichloromethane (J. Renault, S. Giorgi-Renault et al., J. Med. Chem. (1983), 26, 1715-1719).

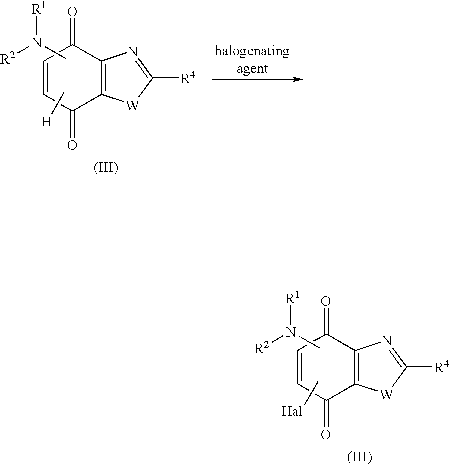

### Diagram C.1a

Preparation of the Intermediates of General Formula (A)

The compounds of general formula (A) in which L, R³, R⁴ and W are as defined above can be obtained, Diagram C.2, from the compounds of general formula (B) in which L, R³, R⁴ and W are as defined above and:

one of Q and Q′ represents an amino or hydroxyl radical and the other represents a hydrogen atom; or

Q and Q′ each represent an amino radical; or

Q and Q′ each represent a hydroxyl radical; or finally

Q and Q′ each represent a methoxy radical.

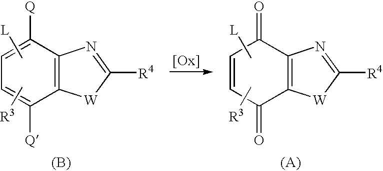

### Diagram C.2

In the case where the compounds of general formula (B) are such that Q and Q′ represent methoxy radicals, the compounds of general formula (A) are obtained by treatment with some cerium(IV) nitrate and ammonium (Beneteau et al., Eur. J. Med. Chem. (1999), 34(12), 1053-1060). In the other cases, the compounds of general formula (A) are obtained by oxidation of the compounds of general formula (B), for example by using FeCl₃ in an acid medium (Antonini et al., Heterocycles (1982), 19(12), 2313-2317) or Fremy's salt (potassium nitrosodisulphonate). (Ryu et al., Bioorg. Med. Chem. Lett. (2000), 10, 461-464), or by using a reagent comprising a hypervalent iodine such as [bis(acetoxy)iodo]benzene or [bis(trifluoroacetoxy)iodo]benzene in aqueous acetonitrile at a temperature preferably comprised between −20° C. and ambient temperature (or approximately 25° C.), and preferably at approximately-5° C. (Kinugawa et al., Synthesis, (1996), 5, 633-636).

In the particular case where L and R³ represent halogen atoms, the compounds of general formula (A) can be obtained, Diagram C.3, by halooxidation of the compounds of general formula (B) in which L and R³ represent hydrogen atoms and Q and/or Q′ is (are) chosen from an amino radical and a hydroxy radical by the action, for example, of potassium or sodium perchlorate in an acid medium (Ryu et al., Bioorg. Med. Chem. Lett. (1999), 9, 1075-1080).

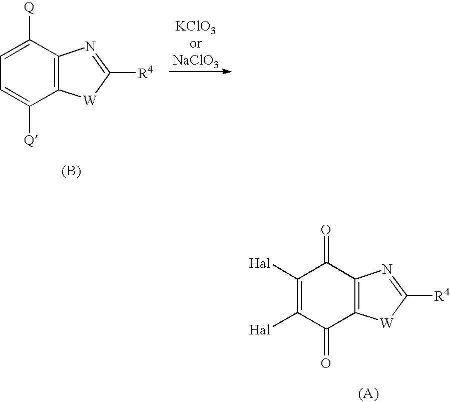

Diagram C.3

Preparation of the Intermediates of General Formula (B)

Certain compounds of general formula (B) in which L, R³, R⁴, Q, Q′ and W are as defined above are industrial products which are known to be available from the usual suppliers.

If they are not commercial and in the particular case where Q or Q′ represents an amino radical, the compounds of general formula (B) can in particular be obtained from nitro derivatives of formula (B.ii) in which Q or Q′ represents a nitro radical by reduction methods which are well known to a person skilled in the art such as, for example hydrogenation in the presence of a palladium catalyst or treatment with tin chloride in hydrochloric acid. If they are not commercial, the compounds of formula (B.ii) can themselves be obtained from the compounds of general formula (B.i) in which the positions corresponding to the Q and Q′ radicals are substituted by hydrogen atoms by nitration methods well known to a person skilled in the art such as, for example, treatment with a mixture of nitric acid and sulphuric acid (cf. Diagram C.4 where only the case in which the compounds of general formula (B) are such that Q=NH₂ and Q′═H is represented).

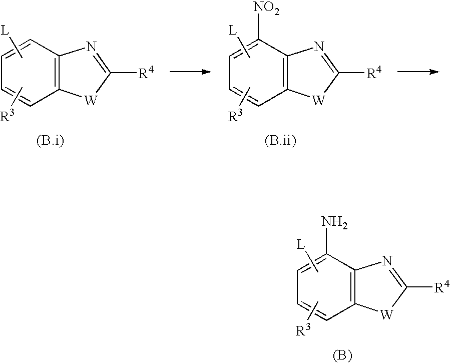

### Diagram C.4

If they are not commercial and in the particular case where R⁴ represents a-H₂—NR²¹R²² radical, the compounds of general formula (B) can be obtained, Diagram C.5, from the compounds of general formula (B.iii) in which R⁴ represents the methyl radical, which is initially subjected to a radical-like reaction using N-bromosuccinimide in the presence of an initiator such as 2.2′-azobis(2-methylpropionitrile) or dibenzoylperoxide in an aprotic solvent such as carbon tetrachloride (CCl₄) at a temperature preferably comprised between ambient temperature (i.e. approximately 25° C.) and 80° C. and under irradiation from a UV lamp (Mylari et al., J. Med. Chem. (1991), 34, 108-122), followed by a substitution of the intermediate of general formula (B.iv) by amines of formula HNR²¹R²² with R²¹ and R²² as defined above.

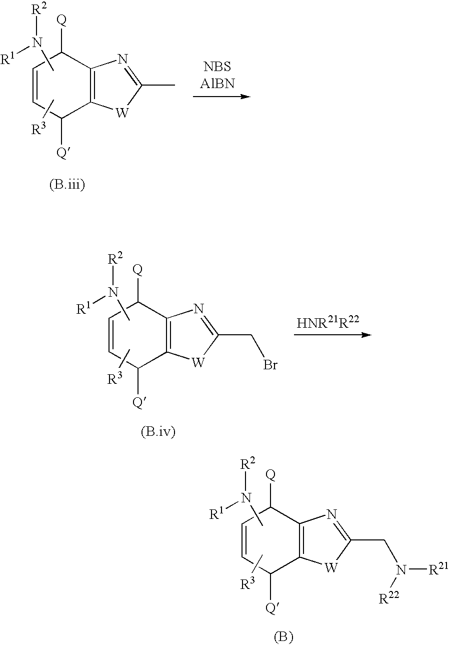

### Diagram C.5

If they are not commercial and in the particular case where R⁴ represents a —CH₂—CO—NR¹⁹R²⁰ radical, the compounds of general formula (B) can be obtained from compounds of general formula (B) in which R⁴ represents the —CH₂—COOH radical, by the standard methods of peptide synthesis (M. Bodansky, The Practice of Peptide Synthesis, 145 (Springer-Verlag, 1984)), for example in tetrahydrofuran, methylene chloride or dimethyl formamide in the presence of a coupling reagent such as cyclohexylcarbodiimide (DCC), 1.1′-carbonyldiimidazole (CDI) (J. Med. Chem. (1992), 35(23), 4464-4472) or benzotriazol-1-yl-oxy-tris-pyrrolidino-phosphonium hexafluorophosphate (PyBOP) (Coste et al., Tetrahedron Lett. (1990), 31, 205).

The compounds of general formula (B) in which R⁴ represents —CH₂—COOH can be obtained from the compounds of general formula (B) in which R⁴ represents the —CH₂—COOR¹⁸ radical in which R¹⁸ represents an alkyl radical by hydrolysis of the ester function under conditions which are known to a person skilled in the art.

In the other cases, the compounds of general formula (B) can be obtained, Diagram C.6, from the compounds of general formula (C) in which L, R³, Q, Q′ and W are as defined above by condensation with the orthoester of general formula R⁴C(OR)₃ in which R is an alkyl radical, for example in the presence of a catalytic quantity of an acid such as, for example, paratoluenesulphonic acid, at a temperature comprised between ambient temperature and 200° C. and preferably at approximately 110° C. (Jenkins et al., J. Org. Chem. (1961), 26, 274) or also in a protic solvent such as ethanol at a temperature comprised between ambient temperature (i.e. approximately 25° C.) and 80° C. and preferably at approximately 60° C. (Scott et al., Synth. Commun. (1989), 19, 2921). A certain number of orthoesters are industrial products which are known to be available from the usual suppliers. The preparation of orthoesters in treating varied nitrile compounds by hydrochloric gas in an alcohol is well known to a person skilled in the art.

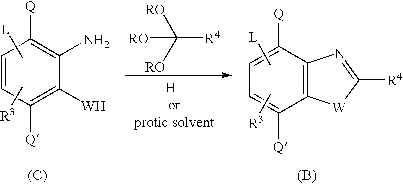

### Diagram C.6

The compounds of general formula (B) in which L, R³, R⁴, Q, Q′ and W are as defined above can also be obtained from the compounds of general formula (C) in which L, R³, Q, Q′ and W are as defined above by condensation of the latter with an acid chloride of formula R⁴—COCl in an inert atmosphere and in a polar and slightly basic solvent such as N-methyl-2-pyrrolidinone (Brembilla et al., Synth. Commun (1990), 20, 3379-3384).

The compounds of general formula (B) in which L, R³, R⁴, Q, Q′ and W are as defined above can also be obtained from the compounds of general formula (C) in which L, R³, Q, Q′ and W are as defined above by condensation with an aldehyde of general formula R⁴—CHO then treatment of the Schiff base obtained with an oxidizing agent such as [bis(acetoxy)iodo]benzene, ferric chloride or dimethylsulphoxide (Racane et al., Monatsh Chem. (1995), 126(12), 1375-1381) or by dehydration with glacial acetic acid at a temperature comprised between ambient temperature (i.e. approximately 25° C.) and 100° C. (Katritzky and Fan, J. Heterocyclic Chem. (1988), 25, 901-906).

The compounds of general formula (B) in which L, R³, R⁴, Q, Q′ and W are as defined above can also be obtained from the compounds of general formula (C) in which L, R³, Q, Q′ and W are as defined above by condensation with a nitrile of general formula R⁴—CN in a mixture of solvents of methanol/glacial acetic acid type at a temperature comprised between ambient temperature (i.e. approximately 25° C.) and 100° C. (Nawwar and Shafik, Collect. Czech Chem. Commun. (1995), 60(12), 2200-2208).

Preparation of the Intermediates of General Formula (C)

Certain compounds of general formula (C) in which L, R³, Q, Q′ and W are as defined above are industrial products which are known to be available from the usual suppliers.

Certain compounds of general formula (C) can be obtained from the compounds of general formula (D)

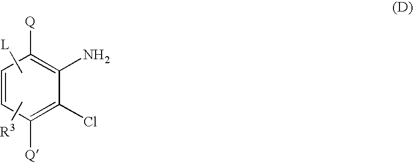

in which L, R³, Q and Q′ are as defined above by reaction, in the case where W represents S, with sodium sulphide hydrated at a temperature comprised between ambient temperature (i.e. approximately 25° C.) and 100° C. (Katritzky and Fan, J. Heterocyclic Chem. (1988), 25, 901-906).

Finally, in the particular case where W represents O, the compounds of general formula (C) are industrial products known to be available from the usual suppliers or which can be synthesized from such products according to the current methods known to a person skilled in the art.

D. Preparation of the Compounds of General Formula (IV)

A certain number of triazolopyrazines of general formula (IV) can be easily prepared according to the procedures described in the U.S. Pat. No. 4,565,815.

The other compounds of general formula (IV) according to the invention can be prepared in a few stages, Diagram D. 1, from the compounds of general formula (IV), in which A′ represents a hydrogen atom or a halogen atom and X′ represents a hydrogen atom or an alkylthio radical. The preparation of the compounds of general formula (III) is described in the U.S. Pat. No. 4,565,815 or in Kobe et al., J. Het. Chem. (1974), 11(2), 199 and s.

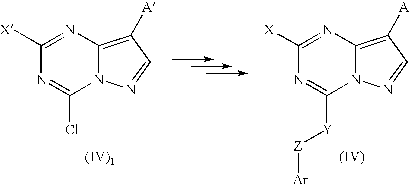

### Diagram D.1

Different cases should be considered according to the nature of the substituents A, X and Y-Z-Ar of the compounds of general formula (IV).

Preparation of the Compounds of General Formula (IV) in Which A Represents a Hydrogen Atom or a Halogen Atom:

Preparation of the Compounds of General Formula (IV) in Which X Represents a Hydrogen Atom or Alkylthio:

In this case, the starting compound of general formula (IV), is such that X represents H or alkylthio and A represents H or a halogen atom Hal. The synthesis strategy is summarised in Diagram D.2 below.

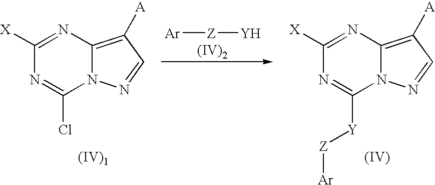

### Diagram D.2

The compound of general formula (IV)₁ is subjected to a nucleophile substitution reaction with the compound of general formula (IV)₂ in order to produce the compound of general formula (IV). The reaction can, if necessary, be carried out in a solvent such as chloroform.

Preparation of the Compounds of General Formula (IV) in Which X Represents an NR⁴R⁵ Radical:

In this case, the starting compound of general formula (IV)₁ is such that X′ represents alkylthio and preferably methylthio. The synthesis strategy is summarised in Diagram D.3 below.

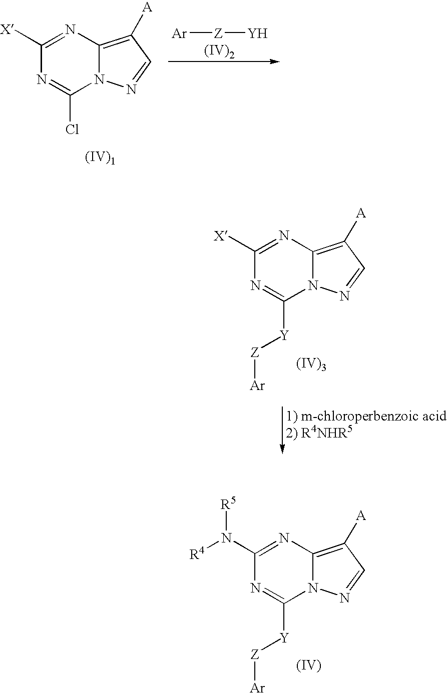

### Diagram D.3

The compound of general formula (IV), is firstly subjected to a substitution reaction with the alcohol or amino of general formula (IV)₂ in order to produce the compound of general formula (IV)₃. The compound of general formula (IV)₃ is then treated with meta-chloroperbenzoic acid then with the amine of general formula R⁴NHR⁵ in order to finally produce the compound of general formula (IV). These reactions are preferably carried out in a solvent such as chloroform.

Preparation of the Compounds of General Formula (IV) in Which X Represents an Alkylthioxo Radical:

This preparation is carried out in a similar way to that described in Diagram D.3, the only difference being that the thioxo derivative is isolated during the second stage instead of being substituted by the amine of general formula R₄NHR⁵ (cf. Diagram D.3a).

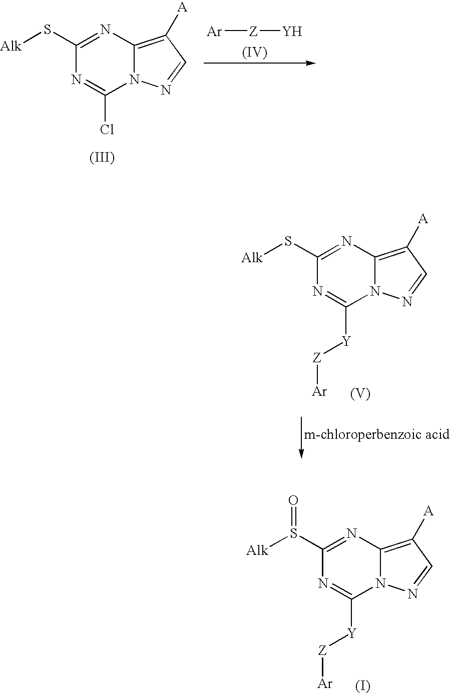

### Diagram D.3a

Preparation of the Compounds of General Formula (I) in Which A Does Not Represent a Hydrogen Atom or a Halogen Atom:

Preparation of the Compounds of General Formula (I) in Which A Represents a —CH₂—NR¹R² Radical:

When A represents an -L-NR¹R² radical in which L represents —CH₂—, the compound of general formula (IV)₄ represented in Diagram D.4 is used for example as starting compound. This compound is a compound of general formula (IV) in which A represents H and its synthesis has therefore been described previously. The compound of general formula (IV)₄ is for example firstly treated with an excess of (chloromethylene)-dimethylammonium chloride in an aprotic polar solvent such as an acetonitrile-dimethylformamide mixture. This allows the compounds of general formula (IV) in which A represents the formyl radical to be obtained. These compounds allow a person skilled in the art to construct through classic chemical reactions different compounds of general formula (IV) with varied A radicals.

In the particular case where A represents an -L-NR₁R² radical in which L represents —CH₂— and R₁ and R² are methyl groups, the compound of general formula (IV) can be directly obtained from the compound of general formula (IV)₄ by reaction with (chloromethylene)-dimethylammonium chloride in excess followed by the action of NaBH₄.

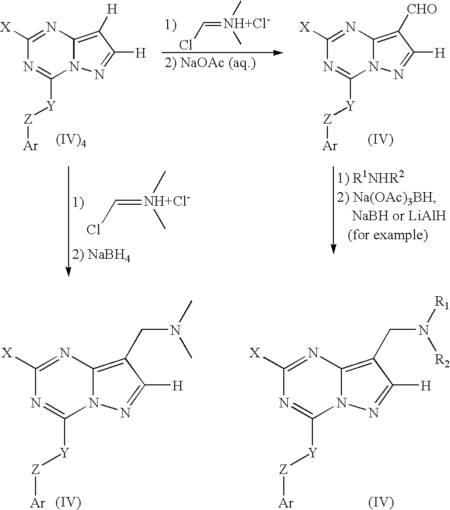

### Diagram D.4

Preparation of the Compounds of General Formula (IV) in Which A Represents an -L-NR₁R² Radical:

These compounds can be prepared in a standard fashion starting from the compound of general formula (IV)₄, for example according to the process represented in Diagram D.5. The compound of general formula (IV)₄ can for example be treated at low temperature (for example at −78° C.) successively with butyllithium in an aprotic polar solvent such as ethyl ether or tetrahydrofuran then the compound of general formula (IV)₅ in which Hal represents a halogen atom, before being hydrolyzed with slightly acidified water in order to produce the compound of general formula (IV) in which A represents an -L-NR₁R² radical.

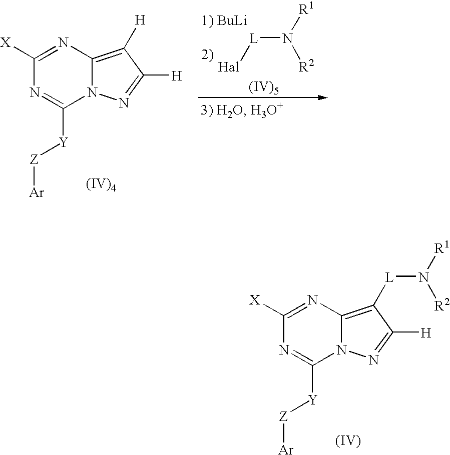

### Diagram D.5

Preparation of the Compounds of General Formula (IV) in Which A Represents an Alkylcarbonyl, Aralkylcarbonyl, Heteroaralkylcarbony Radical:

When it is desired to obtain a compound of general formula (IV) in which A is a —CO-A radical in which A represents an alkyl, aralkyl or heteroaralkyl radical, the compound of general formula (IV)₄ is treated, Diagram D.6, with the compound of general formula Δ-COCl in the presence of AlCl₃ in a suitable solvent, for example in dichloromethane.

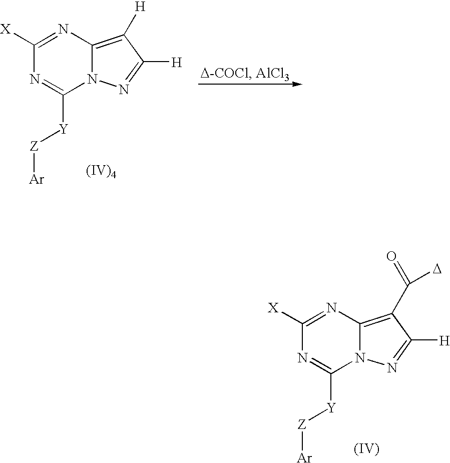

### Diagram D.6

Preparation of the Compounds of General Formula (IV) in Which A Represents a Guanidinoaminomethylenyl or (1,3-dihydro-2-oxoindol)-3-ylidenemethyl Radical:

The compound of general formula (IV) in which A represents a formyl radical is converted to the compound of general formula (IV) in which A represents a guanidinoaminomethylenyl radical, Diagram D.7, by reaction with aminoguanidine bicarbonate in a solvent such as ethanol and in the catalytic presence of a base such as piperidine. The compound of general formula (IV) in which A represents a formyl radical is converted to the compound of general formula (IV) in which A represents a (1,3-dihydro-2-oxoindol)-3-ylidenemethyl radical by the same type of reaction, oxoindole acid replacing aminoguanidine bicarbonate.

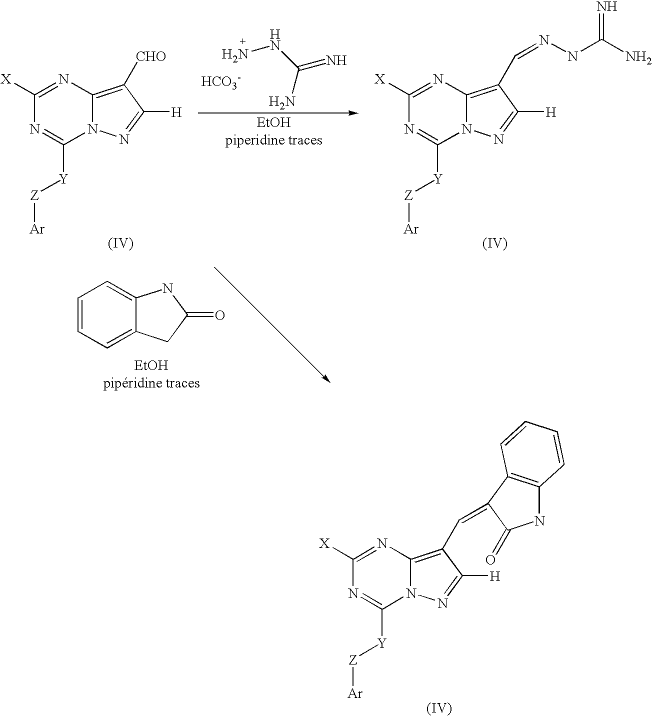

### Diagram D.7

Preparation of the Compounds of General Formula (IV) in Which A Represents a Cyano Radical:

The compound of general formula (IV) in which A represents a formyl radical is converted to the compound of general formula (IV) in which A represents a cyano radical, Diagram D.8, by reaction with hydroxylamine in a mixture of sodium formate and formic acid. The reaction is preferably carried out while heating.

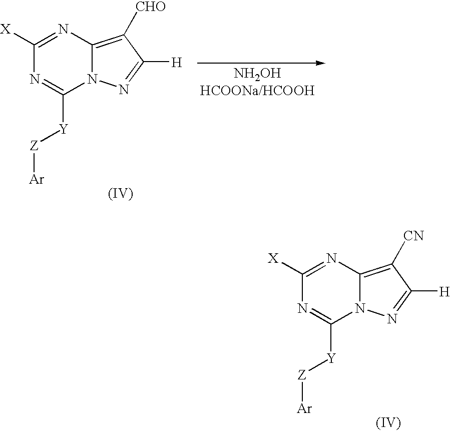

### Diagram D.8

Preparation of the Compounds of General Formula (IV) in Which A Represents a Nitro Radical:

These compounds are easily prepared from compounds of general formula (IV) in which A represents a hydrogen atom by various nitration methods, for example by reacting the latter with a mixture of nitric acid and sulphuric acid or with inorganic nitrate salts in the presence of an acid such as sulphuric acid (cf. Cao et al., Synthesis (1998), 1724). The introduction of the other groups (X and Y-Z-Ar) is carried out, preferably afterwards, using processes similar to those described previously.

E. Preparation of the Compounds of General Formula (V)

These compounds can be prepared using the methods of preparation described in the PCT Patent Application WO 00/39130.

F. Other Compounds

The preparation of these compounds, when they are not commercial, is amply described in the patents and patent applications mentioned and/or the literature and is therefore well known to a person skilled in the art.

Unless they are defined differently, all the technical and scientific terms used here have the same meaning as that usually understood by an ordinary specialist in the field to which this invention belongs. Similarly, all the publications, patent applications, all the patents and all other references mentioned here are incorporated by way of reference.

The following examples are presented to illustrate the above procedures and should in no way be considered to limit the scope of the invention.

### EXAMPLES

### α) Examples 1 to 52

The compounds of the examples 1 to 52 are compounds of general formula (I). The nomenclature used for the examples is in principle in accordance with the IUPAC norms. It was determined using the ACD/Name® software (version 4.53) for Examples 1 to 36 and using the ACD/Name® software (version 5.0) for Examples 37 to 52.

The numbering indicated on the figure below is used for Examples 1 to 36 as regards the positions of substituents —CH₂—R₁, R₂ and R₃ on the polycycles of general sub-formulae (I)₁ and (I)₂:

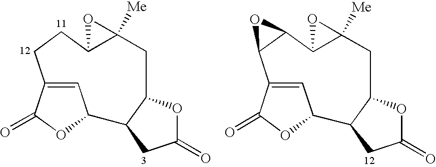

### Example 1

### 12-diisopropylaminomethyl-7-methyl-3,6,10,15-tetraoxapentacyclo [12.2.1.0^{2,4}.0^{5,7}.0^{9,13}]heptadec-1(17)-ene-11,16-dione

Diisopropylamine (500 μmol; 70 μl) is added to a solution of mikanolide (100 μmol; 29 mg) in acetone (1 ml). The reaction mass is stirred for 30 minutes at ambient temperature then the solvent is eliminated by evaporation under reduced pressure. The residue is taken up in ether, filtered and dried under vacuum. 10 mg of product is obtained in the form of a white powder.

NMR-¹H (DMSO): 0.90-1.30 (m, 15H); 1.85 (m, 2H); 2.15 (t, 2H); 3.15-3.50 (m, 4H); 3.95 (s, 1H); 4.75 (m, 1H); 5.50 (s, 1H); 6.00 (s, 1H); 6.25 (s, 1H); 7.60 (s, 1H).

### Example 2

### 12-dimethylamino-3-dimethylaminomethyl-11-hydroxy-8-methyl-5,9,15-trioxatetracyclo[11.2.1.0^{2,6}.0^{8,10}]hexadec-13(16)-ene-4,14-dione:

A solution of dimethylamine (160 μmol; 80 μl; 2M in THF) is added to a solution of mikanolide (30 μmol; 9 mg) in acetone (0.3 ml). The reaction mass is stirred for 30 minutes at ambient temperature then concentrated under reduced pressure. The residue is taken up in ether, filtered and dried under vacuum. 6 mg of expected compound is obtained in the form of a white powder.

NMR-1H (DMSO): 1.11 (s, 3H); 1.94-1.97 (m, 2H); 2.20 (s, 6H); 2.47 (s, 6H); 2.67 (m, 2H); 2.85 (t, 1H); 3.07 (d, 1H); 3.15 (m, 1H); 3.52 (d, 1H); 3.63 (m, 1H); 4.62 (m, 1H); 5.36 (s, 1H); 5.47 (s, 1H); 8.00 (s, 1H).

NMR-¹³C (DMSO): 20.68; 42.85; 43.37; 44, 71; 45.92; 49.95; 57.84; 58.24; 61.61; 62.97; 67.94; 77.09; 80.67; 131.46; 151.01; 172.03; 174.98.

### Example 3

### 12-benzyl(methyl)amino-3-benzyl(methyl)aminomethyl-11-hydroxy-8-methyl-5,9,15-trioxatetracyclo[11.2.1.0^{2,6}.0^{8,10}]hexadec-13(16)-ene-4,14-dione

This compound is obtained by a procedure similar to that described for the synthesis of the, compound of Example 2. The expected product is obtained in the form of a white powder.

NMR-¹H (DMSO): 1.12 (s, 3H); 1.96 (m, 2H); 2.10 (m, 1H); 2.15 (s, 3H); 2.47 (m, 2H); 2.83 (d, 2H); 2.89 (d, 1H); 3.22 (d, 1H), 3.26 (m, 1H); 3.58 (dd, 2H); 3.69 (m, 1H); 3.89 (d, 1H); 3.93 (s, 2H); 4.73 (m, 1H); 5.47 (d, 1H); 5.52 (s, 1H); 7.23-7.40 (m, 10H); 8.10 (s, 1H).

NMR-¹³C (DMSO): 20.65; 39.08; 40.54; 42.09; 43.09; 43.52; 50.18; 56.57; 57.85; 60.17; 61.14; 62.21; 62.33; 68.37; 77.22; 81.01; 126.07; 128.30; 131.48; 138.88; 139.67; 150.35; 172.16; 175.18.

### Example 4

### 11-hydroxy-8-methyl-12-morpholino-3-morpholinomethyl-5,9,15-trioxatetracyclo[11.2.1.0^{2,6}.0^{8,10}]hexadec-13(16)-ene-4,14-dione

This compound is obtained by a procedure similar to that described for the synthesis of the compound of Example 2. The expected product is obtained in the form of a white powder.

NMR-¹H (DMSO): 1.13 (s, 3H); 1,85-2.10 (m, 2H); 2.36 (m, 2H); 2.40 (m, 2H); 2.74 (m, 4H); 2.88 (t, 1H); 2.95 (m, 2H); 3.10 (d, 1H); 3.24 (m, 1H); 3.50-3.70 (m, 10H); 4.64 (m, 1H); 5.49 (s, 1H); 5.50 (d, 1H); 8.01 (s, 1H).

### Example 5

### 12-dimethylamino-11-hydroxy-3,8-dimethyl-5,9,15-trioxatetracyclo[11.2.1.0^{2,6}.0^{8,10}]hexadec-13(16)-ene-4,14-dione:

A solution of dimethylamine (500 μmol, 250 g1, 2M in THF) is added to a solution of dihydromikanolide (100 μmol, 29 mg) in acetone (1 ml). The reaction mass is stirred for 2 hours at ambient temperature then the solvent is eliminated by evaporation under reduced pressure. The residue is taken up in ether, filtered and dried under vacuum. 25 mg of product is obtained in the form of a white powder.

NMR-¹H (DMSO): 1.10 (s, 3H); 1.25 (d, 3H), 1.90 (dd, 1H); 1.99 (t, 1H); 2.49 (s, 6H); 2.58 (t, 1H); 2.94 (m, 1H); 3.06 (d, 1H); 3.51 (m, 1H); 3.63 (m, 1H); 4.62 (m, 1H); 5.34 (s, 1H); 5.37 (d, 1H); 8.00 (s, 1H).

### Example 6

### 11-hydroxy-3,8-dimethyl-4,14-dioxo-5,9,15-trioxatetracyclo[11.2.1.0^{2,6}.0^{8,10}]hexadec-13(16)-ene-12-yl(dimethyl)ammonium maleate

A solution of maleic acid (0.1 mmol; 11.6 mg) in acetone (0.5 ml) is added to a solution of the compound of Example 5 (0.1 mmol; 34 mg) in acetone (0.5 ml). The precipitate is filtered, washed with acetone and dried under reduced pressure. 24 mg of the expected product is obtained in the form of a white powder. Melting point: 178.5° C.

NMR-¹H (DMSO): 1.09 (s, 3H); 1.28 (d, 3H); 1.94 (dd, 1H); 2.05 (m, 1H); 2.63 (t, 1H); 2,70-3.70 (m, 9H); 3.79 (t, 1H); 4.38 (s, 1H); 4.68 (m, 1H); 5.45 (s, 1H); 6.07 (s, 2H); 8.31 (s, 1H).

### Example 7

### 11-hydroxy-3,8-dimethyl-4,14-dioxo-5,9,15-trioxatetracyclo[11.2.1.0^{2,6}.0^{8,10}]hexadec-13(16)-ene-12-yl(dimethyl)ammonium Fumarate of

A solution of fumaric acid (0.1 mmol; 11.6 mg) in acetone (3 ml) is added to a solution of the compound of Example 5 (0.1 mmol; 34 mg) in acetone (0.5 ml). The precipitate is filtered, washed with acetone and dried under reduced pressure. 15 mg of the expected product is obtained in the form of a white powder. Melting point: 159° C.

NMR-¹H (DMSO): 1.11 (s, 3H); 1.25 (d, 3H); 1.92 (dd, 1H); 2.02 (m, 1H); 2.58 (t, 1H); 2.80-4.00 (m, 11H); 4.64 (m, 1H); 5.34 (s, 1H); 6.61 (s, 2H); 8.01 (s, 1H).

### Example 8

### 11-hydroxy-3,8-dimethyl-4,14-dioxo-5,9,15-trioxatetracyclo[11.2.1.0^{2,6}.0^{8,10}]hexadec-13(16)-ene-12-yl(dimethyl)ammonium methane sulphonate

A solution of methanesulphonic acid (0.1 mmol; 1 ml; 0.1N in acetone) is added to a solution of the compound of Example 5 (0.1 mmol; 34 mg) in acetone (2 ml). The precipitate is filtered, washed with acetone and dried under reduced pressure. 24 mg of the expected product is obtained in the form of a white powder. Melting point: 220° C.

NMR-¹H (DMSO): 1.09 (s, 3H); 1.29 (d, 3H); 1.97 (dd, 1H); 2.07 (m, 1H); 2.30 (s, 3H); 2.65 (t, 1H); 2.80-3.15 (m, 7H); 3.28 (d, 1H); 3.85 (t, 1H); 4.66-4.72 (m, 2H); 5.49 (s, 1H); 6.94 (s, 1H); 8.44 (s, 1H); 10.04 (s, 1H).

### Example 9

### 11-hydroxy-3,8-dimethyl-12-(4-methylpiperidino)-5,9,15-trioxatetracyclo[11.2.1.0^{2,6}.0^{8,10}]hexadec-13(16)-ene-4,14-dione

This compound is obtained by a procedure similar to that described for the synthesis of the compound of Example 5. The expected product is obtained in the form of a white powder. Melting point: 210° C.

NMR-¹H (DMSO): 0.80-3.50 (m, 23H); 3.60-3.75 (m, 2H); 4.62 (m, 1H); 5.32 (s, 2H); 8.01 (s, 1H).

### Example 10

### 11-hydroxy-3,8-dimethyl-12-pyrrolidino-5,9,15-trioxatetracyclo[11.2.1.0^{2,6}.0^{8,10}]hexadec-13(16)-ene-4,14-dione

This compound is obtained by a procedure similar to that described for the synthesis of the compound of Example 5. The expected product is obtained in the form of a white powder.

NMR-¹H (DMSO): 1.12 (s, 3H); 1.25 (d, 3H); 1.69 (m, 4H); 1.91 (dd, 1H); 2.00 (m, 1H); 2.60 (t, 1H); 2.80 (m, 4H); 2.95 (m, 1H); 3.02 (d, 1H); 3.45 (s, 1H); 3.63 (m, 1H); 4.61 (m, 1H); 5.34 (s, 1H); 5.42 (d, 1H); 7.97 (s, 1H).

### Example 11

### ethyl 1-[11-hydroxy-3,8-dimethyl-4,14-dioxo-5,9,15-trioxatetracyclo[11.2.1.0^{2,6}.0^{8,10}]hexadec-13(16)-ene-12-yl]-4-piperidinecarboxylate

This compound is obtained by a procedure similar to that described for the synthesis of the compound of Example 5. The expected product is obtained in the form of a white powder.

NMR-¹H (DMSO): 1.00-4.00 (m, 25H); 4.04 (q, 2H); 4.64 (m, 1H); 5.35 (s, 1H); 5.48 (d, 1H); 8.07 (s, 1H).

### Example 12

### 12-(4-benzylpiperidino)-11-hydroxy-3,8-dimethyl-5,9,15-trioxatetracyclo[11.2.1.0^{2,6}.0^{8,10}]hexadec-13(16)-ene-4,14-dione

This compound is obtained by a procedure similar to that described for the synthesis of the compound of Example 5. The expected product is obtained in the form of a white powder.

NMR-¹H (DMSO): 1.00-1.80 (m, 12H); 1.85-2.10 (m, 2H); 2.354.00 (m, 6H); 4.63 (m, 1H); 5.33 (m, 2H); 7.00-7.20 (m, 5H); 8.03 (s, 1H).

### Example 13

### 11-hydroxy-3,8-dimethyl-12-piperidino-5,9,15-trioxatetracyclo[11.2.1.0^{2,6}.0^{8,10}]hexadec-13(16)-ene-4,14-dione

This compound is obtained by a procedure similar to that described for the synthesis of the compound of Example 5. The expected product is obtained in the form of a white powder.

NMR-¹H (DMSO): 1.11 (s, 3H); 1.26 (d, 3H); 1,35-1.70 (m, 6H); 1,85-2.14 (m, 2H); 2,57-3.18 (m, 7H); 3,50-3.75 (m, 2H); 4.64 (m, 1H); 5.34 (m, 2H); 8.04 (s, 1H).

### Example 14

### 12-(1,4-dioxa-8-azaspiro[4.5]dec-8-yl)-11-hydroxy-3,8-dimethyl-5,9,15-trioxatetracyclo[11.2.1.0^{2,6}.0^{8,10}]hexadec-13(16)-ene-4,14-dione

This compound is obtained by a procedure similar to that described for the synthesis of the compound of Example 5. The expected product is obtained in the form of a white powder.

NMR-¹H (DMSO): 1.11 (s, 3H); 1.26 (d, 3H); 1.40-1.80 (m, 6H); 1.85-2.05 (m, 2H); 2.58-4.00 (m, 17H); 4.67 (m, 1H); 5.37 (s, 1H); 5.44 (d, 1H); 8.08 (s, 1H).

### Example 15

### 11-hydroxy-3,8-dimethyl-12-morpholino-5,9,15-trioxatetracyclo[11.2.1.0^{2,6}.0^{8,10}]hexadec-13(16)-ene-4,14-dione

This compound is obtained by a procedure similar to that described for the synthesis of the compound of Example 5. The expected product is obtained in the form of a white powder.

NMR-¹H (DMSO): 1.10 (s, 3H); 1.25 (d, 3H); 1.89 (dd, 1H); 2.01 (m, 1H); 2.61 (t, 1H); 2.75 (m, 2H); 3.95 (m, 3H); 3.08 (d, 1H); 3.55-3.75 (m, 5H); 4.63 (1H); 5.33 (s, 1H); 5.54 (d, 1H); 8.04 (s, 1H).

### Example 16

### 11-(tert-butyldimethylsiloxy)-12-dimethylamino-3,8-dimethyl-5,9,15-trioxatetracyclo[11.2.1.0^{2,6}.0^{8,10}]hexadec-13(16)-ene-4,14-dione

Terbutyldimethylsilyl chloride (80 μmol, 12 mg) is added to a solution of the compound of Example 5 (80 μmol; 27 mg) and imidazole (160 μmol; 11 mg) in DMF (0.5 ml). The solution obtained is stirred for 20 hours then the reaction mass is poured into water. The aqueous phase is extracted twice with ethyl acetate, the organic phase is washed with water then with a solution of sodium chloride. The organic phase is dried over magnesium sulphate, filtered then evaporated. The residue is eluted on silica with an isopropyl acetate and dichloromethane mixture of (20/80). 20 mg of product is obtained in the form of a white powder.

NMR-¹H (DMSO): 0.04 (s, 3H); 0.07 (s, 3H); 0.89 (s, 9H); 1.14 (s, 3H); 1.25 (d, 3H); 1.90 (dd, 1H); 1.99 (dd, 1H); 2.48 (s, 6H); 2.63 (t, 1H); 2.93-2.98 (m, 1H); 3.12 (d, 1H); 3.43 (m, 1H); 3.80 (m, 1H); 4.61 (m, 1H); 5.36 (s, 1H); 8.03 (s, 1H).

### Example 17

### 3,8-dimethyl-12-(4-methylpiperidino)-4,14-dioxo-5,9,15-trioxatetracyclo[11.2.1.0^{2,6}.0^{8,10}]hexadec-13(16)-en-11-yl Acetate

Acetic anhydride (150 μmol; 15 μl) is added to a solution of the compound of Example 9 (100 μmol; 40 mg) in pyridine (0.5 ml). The solution obtained is stirred for 20 hours then the reaction mass is poured into water. The aqueous phase is extracted twice with some ethyl acetate and the organic phase obtained is washed with water then with a solution of sodium chloride. The organic phase is dried over sulphate of magnesium, filtered then evaporated. The residue is eluted on silica with an isopropyl acetate and dichloromethane mixture (20/80). 16 mg of product is obtained in the form of a white powder.

NMR-¹H (DMSO): 0.90 (d, 3H); 1.11 (s, 3H); 1.26 (d, 3H); 1.35 (m, 1H); 1.60 (m, 2H), 1.94 (dd, 1H); 2.03 (d, 1H); 2.09 (s, 3H); 2.43 (t, 1H); 2.60 (t, 1H); 2.98 (d, 1H); 2.94-3.05 (m, 2H); 3,36-3.45 (m, 4H); 4.07 (d, 1H); 4.64 (dd, 1H); 4.70 (m, 1H); 5.38 (s, 1H); 8.12 (s, 1H).

### Example 18

### 3,8-dimethyl-12-(4-methylpiperidino)-4,14-dioxo-11-phenylcarbonyloxy-5,9,15-trioxatetracyclo[11.2.1.0^{2,6}.0^{8,10}]hexadec-13(16)-ene

Benzoyl chloride (400 μmol; 46 μl) is added to a solution of the compound of Example 9 (100 μmol; 40 mg) in pyridine (0.5 ml). The reaction mass is stirred for 2 hours then treated in the same way as for the preparation of the compound of Example 17. 25 mg of product is obtained in the form of a white powder. Melting point: 234° C.

NMR-¹H (DMSO): 0.73 (d, 3H); 1.18 (s, 3H); 1.25 (m, 1H); 1.27 (d, 3H); 1.45-1.60 (m, 2H); 2.00 (dd, 1H); 2.10 (m, 1H); 2.65 (t, 1H); 2,92-3.15 (m, 3H); 3.45 (m, 2H); 3.54 (d, 1H); 4.18 (d, 1H); 4.36 (t, 1H); 4.74 (m, 1H); 4.95 (t, 1H); 5.41 (s, 1H); 7.58 (t, 2H); 7.70 (t, 1H); 8.01 (d, 2H); 8.19 (s, 1H).

### Example 19

### ethyl 3,8-dimethyl-12-(4-methylpiperidino)-4,14-dioxo-5,9,15-trioxatetracyclo[11.2.1.0^{2,6}.0^{8,10}]hexadec-13(16)-en-11-ylcarbonate

Ethyl chloroformate (300 μmol; 28 μl) is added to a solution of the compound of Example 9 (100 μmol; 40 mg) in pyridine (0.5 ml). The reaction mass is stirred for 2 hours then treated in the same way as for the preparation of the compound of Example 17. 20 mg of product is obtained in the form of a white powder.

NMR-¹H (DMSO): 0.88 (d, 3H); 1.10-1.40 (m, 12H); 1.59 (m, 2H); 2.90-2.10 (m, 2H); 2.35-2.50 (m, 2H); 2.58 (t, 1H); 2.80 (d, 1H); 2.95-3.07 (m, 2H); 3.40 (d, 1H); 4,11-4.25 (m, 3H); 4.43 (dd, 1H); 4.70 (m, 1H); 5.39 (s, 1H); 8.13 (s, 1H).

### Example 20

### 11-hydroxy-12-isobutylsulphanyl-3-isobutylsulphanylmethyl-8-methyl-5,9,15-trioxatetracyclo[11.2.1.0^{2,6}.0^{8,10}]hexadec-13(16)-ene-4,14-dione

2-methyl-1-propanethiol (500 μmol; 54 μl) is added to a solution of mikanolide (100 μmol; 30 mg) and dimethylaminopyridine (10 μmol; 1.2 mg) in acetone (1 ml). The reaction mass is stirred for two hours at ambient temperature then the solvent is evaporated off under reduced pressure. The residue is taken up in ether, the precipitate is filtered, washed with ether and dried under vacuum. 35 mg of product is obtained in the form of a white powder.

NMR-¹H (DMSO): 0.96 (m, 12H); 1.15 (s, 3H); 1.77 (m, 2H); 1.93 (d, 2H); 2.50 (m, 4H); 2.80-2.98 (m, 4H); 3.39 (m, 1H); 3.76 (m, 1H); 4.07 (d, 1H); 4.62 (q, 1H); 5.52 (s, 1H); 5.62 (s, 1H); 8.06 (s, 1H).

### Example 21

### 11-hydroxy-12-isobutylsulphanyl-3,8-dimethyl-5,9,15-trioxatetracyclo[11.2.1.0^{2,6}.0^{8,10}]hexadec-13(16)-ene-4,14-dione

2-methyl-1-propanethiol (500 μmol; 54 μl) is added to a solution of dihydromikanolide (100 μmol; 30 mg) and dimethylaminopyridine (10 μmol; 1.2 mg) in acetone (1 ml). The reaction mass is stirred for two hours at ambient temperature then the solvent is evaporated off under reduced pressure. The residue is taken up in ether then the precipitate formed is filtered, washed with ether and dried under vacuum. 25 mg of product is obtained in the form of a white powder.

NMR-¹H (DMSO): 0.96 (t, 6H); 1.13 (s, 3H); 1.25 (d, 3H); 1.78 (m, 1H); 1.89 (dd, 1H); 2.00 (t, 1H); 2.48 (m, 2H); 2.62 (t, 1H); 2.82 (d, 1H), 2.98 (m, 1H); 3.78 (m, 1H); 4.07 (d, 1H); 4.57 (m, 1H); 5.40 (s, 1H); 5.61 (d, 1H); 8.06 (s, 1H).

### Example 22

### 12-(dimethylamino)-3,8-dimethyl-4,14-dioxo-5,9,15-trioxatetracyclo[11.2.1.0^{2,6}.0^{8,10}]hexadec-13(16)-en-11-yl Benzoate

This compound is obtained by a procedure similar to that described for the synthesis of the compound of Example 18. The expected product is obtained in the form of a white powder.

NMR-¹H (DMSO): 1.21 (s, 3H); 1.28 (d, 3H); 1.98 (dd, 1H); 2.08 (t, 1H); 2.48 (s, 6H); 2.66 (t, 1H); 3.01 (m, 1H); 3.51 (d, 1H); 4.04 (d, 1H); 4.71 (m, 1H); 5.06 (dd, 1H); 4.43 (s, 1H); 7.58 (m, 2H); 7.70 (t, 1H); 8.01 (d, 2H); 8.20 (s, 1H).

### Example 23

### 12-(dimethylamino)-3,8-dimethyl-4,14-dioxo-5,9,15-trioxatetracyclo[11.2.1.0^{2,6}.0^{8,10}]hexadec-13(16)-en-11-yl Acetate

This compound is obtained by a procedure similar to that described for the synthesis of the compound of Example 17. The expected product is obtained in the form of a white powder.

NMR-¹H (DMSO): 1.14 (s, 3H); 1.26 (d, 3H); 1.94 (dd, 1H); 2.05 (t, 1H); 2.08 (s, 3H); 2.45 (s, 6H); 2.62 (t, 1H); 3.97 (m, 1H); 3.32 (m, 1H); 3.90 (d, 1H); 4.68 (m, 1H); 4.78 (m, 1H); 5.39 (s, 1H); 8.12 (s, 1H).

### Example 24

### 12-(dimethylamino)-3,8-dimethyl-4,14-dioxo-5,9,15-trioxatetracyclo[11.2.1.0^{2,6}.0^{8,10}]hexadec-13(16)-en-11-yl Cyclohexanecarboxylate

This compound is obtained by a procedure similar to that described for the synthesis of the compound of Example 18. The expected product is obtained in the form of a white powder.

NMR-¹H (DMSO): 1.14 (s, 3H); 1.26 (d, 3H); 1.08-1.48 (m, 5H); 1.58 (m, 1H); 1.68 (m, 2H); 1.84 (t, 2H); 1.93 (dd, 1H); 2.03 (t, 1H); 2.37 (m, 1H); 2.44 (s, 6H); 2.61 (t, 1H); 2.98 (m, 1H); 3.32 (t, 1H); 3.87 (d, 1H); 4.66 (m, 1H); 4.77 (dd, 1H); 5.40 (s, 1H); 8.12 (s, 1H).

### Example 25

### 12-(dimethylamino)-3,8-dimethyl-4,14-dioxo-5,9,15-trioxatetracyclo[11.2.1.0^{2,6}.0^{8,10}]hexadec-13(16)-en-11-yl 4-fluorobenzoate

This compound is obtained by a procedure similar to that described for the synthesis of the compound of Example 18. The expected product is obtained in the form of a white powder.

NMR-¹H (DMSO): 1.20 (s, 3H); 1.28 (d, 3H); 1.97 (dd, 1H); 2.08 (t, 1H); 2.46 (s, 6H); 2.65 (t, 1H); 3.00 (m, 1H); 3.50 (d, 1H); 4.04 (d, 1H); 4.71 (m, 1H); 5.04 (dd, 1H); 5.43 (s, 1H); 7.41 (t, 2H); 8.06 (dd, 2H); 8.20 (s, 1H).

### Example 26

### 11-{[tert-butyl(dimethyl)silyl]oxy}-12-(dimethylamino)-3,8-dimethyl-5,9,15-trioxatetracyclo[11.2.1.0^{2,6}.0^{8,10}]hexadec-13(16)-ene-4,14-dione Hydrochloride

A solution of hydrochloric acid (0.3 mmol; 0.3 ml; 1N in ether) is added to a solution of the compound of Example 16 (0.22 mmol; 100 mg) in acetone (2 ml). The precipitate is filtered, washed with a little acetone, with ether and dried under reduced pressure. 70 mg of expected product is obtained in the form of a white powder.

NMR-¹H (DMSO): 0.14 (d, 6H); 0.90 (s, 9H); 1.15 (s, 3H); 1.27 (d, 3H); 1.85 (dd, 1H); 2.05 (t, 1H); 2.72 (t, 1H); 2.90-3.25 (m, 7H); 3.72 (m, 1H); 3.93 (m, 1H); 4.76 (m, 2H); 5.46 (s, 1H); 8.70 (d, 1H); 11.64 (s, 1H).

### Example 27

### 12-(dimethylamino)-3,8-dimethyl-4,14-dioxo-5,9,15-trioxatetracyclo[11.2.1.0^{2,6}.0^{8,10}]hexadec-13(16)-en-11-yl Heptanoate

This compound is obtained by a procedure similar to that described for the synthesis of the compound of Example 18. The expected product is obtained in the form of a white powder.

NMR-¹H (DMSO): 0.86 (t, 3H); 1.14 (s, 3H); 1.20-1.35 (m, 9H); 1.55 (m, 2H); 1.95 (dd, 1H); 2.02 (t, 1H); 2.35 (t, 2H); 2.44 (s, 6H); 2.61 (t, 1H); 2.96 (m, 1H); 3.33 (t, 1H); 3.89 (d, 1H); 4.68 (m, 1H); 4.77 (dd, 1H); 5.40 (s, 1H); 8.12 (s, 1H).

### Example 28

### 12-(dimethylamino)-3,8-dimethyl-4,14-dioxo-5,9,15-trioxatetracyclo [11.2.1.0^{2,6}.0^{8,10}]hexadec-13(16)-en-11-yl 4-(trifluoromethyl)benzoate

This compound is obtained by a procedure similar to that described for the synthesis of the compound of Example 18. The expected product is obtained in the form of a white powder.

NMR-¹H (DMSO): 1.21 (s, 3H); 1.28 (d, 3H); 2.01 (dd, 1H); 2.06 (t, 1H); 2.48 (s, 6H); 2.66 (t, 1H); 3.00 (m, 1H); 3.55 (d, 1H); 4.09 (d, 1H); 4.73 (m, 1H); 5.04 (dd, 1H); 5.44 (s, 1H); 7.96 (d, 2H); 8.19 (d, 2H); 8.21 (s, 1H).

### Example 29

### 12-(dimethylamino)-3,8-dimethyl-4,14-dioxo-5,9,15-trioxatetracyclo[11.2.1.0^{2,6}.0^{8,10}]hexadec-13(16)-en-11-yl 2-thiophenecarboxylate

This compound is obtained by a procedure similar to that described for the synthesis of the compound of Example 18. The expected product is obtained in the form of a white powder.

NMR-¹H (DMSO): 1.20 (s, 3H); 1.27 (d, 3H); 1.99 (m, 1H); 2.07 (t, 1H); 2.49 (s, 6H); 2.65 (t, 1H); 3.00 (m, 1H); 3.47 (d, 1H); 4.00 (d, 1H); 4.70 (m, 1H); 5.01 (dd, 1H); 5.43 (s, 1H); 7.26 (t, 1H); 7.87 (d, 1H); 8.01 (dd, 1H); 8.18 (s, 1H).

### Example 30

### 12-(dimethylamino)-3,8-dimethyl-4,14-dioxo-5,9,15-trioxatetracyclo[11.2.1.0^{2,6}.0^{8,10}]hexadec-13(16)-en-11-yl 3,3-dimethylbutanoate

This compound is obtained by a procedure similar to that described for the synthesis of the compound of Example 18. The expected product is obtained in the form of a white powder.

NMR-¹H (DMSO): 1.00 (s, 9H); 1.15 (s, 3H); 1.26 (d, 3H); 1.94 (dd, 1H); 2.03 (t, 1H); 2.24 (dd, 2H); 2.45 (s, 6H); 2.62 (t, 1H); 2.98 (m, 1H); 3.32 (d, 1H); 3.86 (d, 1H); 4.65 (m, 1H); 4.81 (dd, 1H); 5.40 (s, 1H); 8.12 (s, 1H).

### Example 31

### 12-(dimethylamino)-3,8-dimethyl-4,14-dioxo-5,9,15-trioxatetracyclo[11.2.1.0^{2,6}.0^{8,10}]hexadec-13(16)-en-11-yl 1-benzothiophene-2-carboxylate

This compound is obtained by a procedure similar to that described for the synthesis of the compound of Example 18. The expected product is obtained in the form of a white powder.

NMR-¹H (DMSO): 1.22 (s, 3H); 1.28 (d, 3H); 2.01 (dd, 1H); 2.08 (m, 1H); 2.50 (s, 6H); 2.66 (t, 1H); 3.00 (m, 1H); 3.52 (d, 1H); 4.05 (d, 1H); 4.71 (m, 1H); 5.06 (dd, 1H); 5.44 (s, 1H); 7.50 (t, 1H); 7.56 (t, 1H); 8.09 (t, 2H); 8.21 (s, 1H); 8.27 (s, 1H).

### Example 32

### 12-(dimethylamino)-3,8-dimethyl-4,14-dioxo-5,9,15-trioxatetracyclo[11.2.1.0^{2,6}.0^{8,10}]hexadec-13(16)-en-11-yl 2-furoate

This compound is obtained by a procedure similar to that described for the synthesis of the compound of Example 18. The expected product is obtained in the form of a white powder.

NMR-¹H (DMSO): 1.19 (s, 3H); 1.27 (d, 3H); 1.97 (dd, 1H); 2.07 (t, 1H); 2.47 (s, 6H); 2.64 (t, 1H); 3.00 (m, 1H); 3.46 (d, 1H); 4.00 (d, 1H); 4.70 (m, 1H); 4.98 (dd, 1H); 5.43 (s, 1H); 6.72 (d, 1H); 7.36 (d, 1H); 8.03 (s, 1H); 8.18 (s, 1H).

### Example 33

### 12-(dimethylamino)-3,8-dimethyl-4,14-dioxo-5,9,15-trioxatetracyclo[11.2.1.0^{2,6}.0^{8,10}]hexadec-13(16)-en-11-yl 5-nitro-2-furoate

This compound is obtained by a procedure similar to that described for the synthesis of the compound of Example 18. The expected product is obtained in the form of a white powder.

NMR-¹H (DMSO): 1.19 (s, 3H); 1.28 (d, 3H); 1.98 (dd, 1H); 2.08 (t, 1H); 2.45 (s, 6H); 2.64 (t, 1H); 3.00 (m, 1H); 3.53 (d, 1H); 4.08 (d, 1H); 4.72 (m, 1H); 4.97 (dd, 1H); 5.44 (s, 1H); 7.65 (d, 1H); 7.80 (d, 1H); 8.21 (s, 1H).

### Example 34

### 2-thiophenecarboxylate of 12-(dimethylamino)-3,8-dimethyl-4,14-dioxo-5,9,15-trioxatetracyclo[11.2.1.0^{2,6}.0^{8,10}]hexadec-13(16)-en-11-yl Hydrochloride

A solution of hydrochloric acid (0.8 mmol; 0.8 ml; 1N in ether) is added to a solution of the compound of Example 29 (0.44 mmol; 196 mg) in acetone (4 ml). The precipitate is filtered, washed with a little acetone, with ether and dried under reduced pressure. 180 mg of the expected product is obtained in the form of a white powder.

NMR-¹H (DMSO): 1.23 (s, 3H); 1.29 (d, 3H); 1.90 (dd, 1H); 2.13 (t, 1H); 2.76 (t, 1H); 2,85-3.25 (m, 7H); 3.95 (m, 1H); 4.78 (m, 1H); 5.02 (m, 1H); 5.38 (m, 1H); 5.51 (s, 1H); 7.29 (t, 1H); 7.97 (s, 1H); 8.08 (d, 1H); 8.86 (s, 1H); 12.12 (s, 1H).

### Example 35

### 12-(dimethylamino)-3,8-dimethyl-4,14-dioxo-5,9,15-trioxatetracyclo[11.2.1.0^{2,6}.0^{8,10}]hexadec-13(16)-en-11-yl 2-thienylacetate

This compound is obtained by a procedure similar to that described for the synthesis of the compound of Example 18. The expected product is obtained in the form of a white powder.

NMR-¹H (DMSO): 1.14 (s, 3H); 1.26 (d, 3H); 1.94 (dd, 1H); 2.04 (m, 1H); 2.38 (s, 6H); 2.61 (t, 1H); 2.97 (m, 1H); 3.37 (d, 1H); 3.88 (d, 1H); 4.00 (d, 2H); 4.68 (m, 1H); 4.78 (dd, 1H); 5.39 (s, 1H); 6.98 (m, 2H); 7.43 (d, 1H); 8.12 (s, 1H).

### Example 36

### 12-(dimethylamino)-3,8-dimethyl-4,14-dioxo-5,9,15-trioxatetracyclo[11.2.1.0^{2,6}.0^{8,10}]hexadec-13(16)-en-11-yl phenoxyacetate

This compound is obtained by a procedure similar to that described for the synthesis of the compound of Example 18. The expected product is obtained in the form of a white powder.

NMR-¹H (DMSO): 1.23 (s, 3H); 1.35 (d, 3H); 2.04 (dd, 1H); 2.13 (t, 1H); 2.58 (s, 6H); 2.67 (t, 1H); 3.06 (m, 1H); 3.48 (d, 1H); 4.08 (d, 1H); 4.77 (m, 1H); 4.86 (m, 1H); 4.96 (dd, 2H); 5.50 (s, 1H); 7.05 (m, 3H); 7.38 (m, 2H); 8.23 (s, 1H).

### Example 37

### 8-(dimethylamino)-3,10a-dimethyl-2,6dioxodecahydro-4,7-methenofuro[3,2-c]oxireno[f]oxacycloundecin-9-yl 4-tert-butylphenylcarbamate

4-terbutylphenylisocyanate (250 μmol; 44 mg) is added to a solution of the compound of Example 5 (200 μmol; 67 mg) in 1,2-dichloroethane (10 ml). The solution obtained is stirred for 20 hours at 60° C. before evaporating the solvent under reduced pressure. The residue is eluted on silica using an acetone and dichloromethane mixture (20/80). The residue is taken up in ether, filtered and dried under vacuum. 36 mg of product is obtained in the form of a white powder.

NMR-¹H (DMSO): 1.18 (s, 3H); 1.25 (s, 9H); 1.27 (d, 3H); 1.95 (dd, 1H); 2.08 (t, 1H); 2.48 (s, 6H); 2.64 (t, 1H); 2.98 (m, 1H); 3.37 (m, 1H); 3.91 (d, 1H); 4.69 (m, 1H); 4.80 (dd, 1H); 5.40 (s, 1H); 7.29 (d, 2H); 7.38 (d, 2H); 8.12 (s, 1H); 9.67 (s, 1H).

### Example 38

### 8-(dimethylamino)-3,10a-dimethyl-2,6-dioxodecahydro-4,7-methenofuro[3,2-c]oxireno[f]oxacycloundecin-9-yl thien-2-ylcarbamate

This compound is obtained by a procedure similar to that described for the synthesis of the compound of Example 18. The expected product is obtained in the form of a white powder.

NMR-¹H (DMSO): 1.18 (s, 3H); 1.26 (d, 3H); 1.95 (m, 1H); 2.06 (m, 1H); 2.48 (s, 6H); 2.64 (t, 1H); 2.97 (m, 1H); 3.36 (m, 1H); 3.90 (m, 1H); 4.68 (m, 1H); 4.79 (m, 1H); 5.40 (s, 1H); 6.61 (s, 1H); 6.82 (s, 1H); 6.94 (s, 1H); 8.13 (s, 1H); 10.78 (s, 1H).

### Example 39

### 8-(dimethylamino)-3,10a-dimethyl-2,6-dioxodecahydro-4,7-methenofuro[3,2-c]oxireno[f]oxacycloundecin-9-yl 2-methoxyphenylcarbamate

This compound is obtained by a procedure similar to that described for the synthesis of the compound of Example 18. The expected product is obtained in the form of a white powder.

NMR-¹H (DMSO): 1.17 (s, 3H); 1.27 (d, 3H); 1.94 (dd, 1H); 2.05 (t, 1H); 2.48 (s, 6H); 2.63 (t, 1H); 2.98 (m, 1H); 3.37 (m, 1H); 3.80 (s, 3H); 3.87 (d, 1H); 4.68 (m, 1H); 4.80 (dd, 1H); 5.40 (s, 1H); 6.90 (t, 1H); 7.02 (d, 1H); 7.09 (t, 1H); 7.59 (d, 1H); 8.12 (s, 1H); 8.59 (s, 1H).

### Example 40

### 8-(dimethylamino)-3,10a-dimethyl-2,6dioxodecahydro-4,7-methenofuro[3,2-c]oxireno[f]oxacycloundecin-9-yl 2(methylthio)phenylcarbamate

This compound is obtained by a procedure similar to that described for the synthesis of the compound of Example 18. The expected product is obtained in the form of a white powder.

NMR-¹H (DMSO): 1.17 (s, 3H); 1.27 (d, 3H); 1.95 (dd, 1H); 2.08 (t, 1H); 2.40 (s, 3H); 2.48 (s, 6H); 2.63 (t, 1H); 2.98 (m, 1H); 3.37 (s, 1H); 3.84 (d, 1H); 4.67 (m, 1H); 4.80 (dd, 1H); 5.39 (s, 1H); 7.15-7.25 (m, 3H); 7.32 (t, 1H); 8.10 (s, 1H); 8.90 (s, 1H).

### Example 41

### 8-(dimethylamino)-3,10a-dimethyl-2,6-dioxodecahydro-4,7-methenofuro[3,2-c]oxireno[f]oxacycloundecin-9-yl 2-ethoxyphenylcarbamate

This compound is obtained by a procedure similar to that described for the synthesis of the compound of Example 18. The expected product is obtained in the form of a white powder.

NMR-¹H (DMSO): 1.17 (s, 3H); 1.27 (d, 3H); 1.35 (t, 3H); 1.95 (dd, 1H); 2.05 (t, 1H); 2.48 (s, 6H); 2.63 (t, 1H); 2.98 (m, 1H); 3.34 (m, 1H); 3.90 (d, 1H); 4.07 (q, 2H); 4.67 (m, 1H); 4.79 (dd, 1H); 5.40 (s, 1H); 6.90 (t, 1H); 7.01 (d, 1H); 7.07 (t, 1H); 7.58 (d, 1H); 8.12 (s, 1H); 8.46 (s, 1H).

### Example 42

### 8-(dimethylamino)-3,10a-dimethyl-2,6-dioxodecahydro-4,7-methenofuro[3,2-c]oxireno[f]oxacycloundecin-9-yl thien-3-ylcarbamate

This compound is obtained by a procedure similar to that described for the synthesis of the compound of Example 18. The expected product is obtained in the form of a white powder.

NMR-¹H (DMSO): 1.18 (s, 3H); 1.27 (d, 3H); 1.95 (dd, 1H); 2.05 (m, 1H); 2.48 (s, 6H); 2.64 (t, 1H); 2.98 (m, 1H); 3.36 (m, 1H); 3.90 (d, 1H); 4.68 (m, 1H); 4.80 (dd, 1H); 5.40 (s, 1H); 7.04 (d, 1H); 7.22 (s, 1H); 7.43 (t, 1H); 8.12 (s, 1H); 10.08 (s, 1H).

### Example 43

### 8-(dimethylamino)-3,10a-dimethyl-2,6-dioxodecahydro-4,7-methenofuro[3,2-c]oxireno[f]oxacycloundecin-9-yl 1-benzothien-3-ylcarbamate

This compound is obtained by a procedure similar to that described for the synthesis of the compound of Example 18. The expected product is obtained in the form of a white powder.

NMR-¹H (DMSO): 1.20 (s, 3H); 1.28 (d, 3H); 1.98 (dd, 1H); 2.07 (m, 1H); 2.48 (s, 6H); 2.65 (t, 1H); 2.98 (m, 1H); 3.40 (m, 1H); 3.97 (d, 1H); 4.70 (m, 1H); 4.82 (dd, 1H); 5.40 (s, 1H); 7.40 (m, 2H); 7.65 (s, 1H); 7.95 (d, 1H); 8.14 (m, 2H); 10.00 (s, 1H).

### Example 44

### 8-(dimethylamino)-3,10a-dimethyl-2,6-dioxodecahydro-4,7-metheno-[3,2-c]oxireno[f]oxacycloundecin-9-yl N-(ter-butoxycarbonyl)glycinate

1-(3-dimethylaminopropyl)-3-ethylcarbodiimide hydrochloride (200 μmol; 38 mg), N-terbutyloxycarbonylglycine (200 μmol; 35 mg), triethylamine (200 μmol; 28 μl) and dimethylaminopyridine (10 mol; 3 mg) is added to a solution of the compound of Example 5 (200 μmol; 60 mg) in dichloromethane (5 ml). The solution is stirred for three hours at ambient temperature, poured into a solution of NaHCO₃ then extracted with ethyl acetate. The organic phase is washed with water then with a saturated solution of sodium chloride before being dried over MgSO₄ and filtered. The solvent is eliminated by distillation under reduced pressure. The residue is eluted on silica using an acetone and dichloromethane mixture (40/60). The residue is taken up in ether, filtered and dried under vacuum. 25 mg of product is obtained in the form of a white powder.

NMR-¹H (DMSO): 1.15 (s, 3H); 1.26 (d, 3H); 1.39 (s, 9H); 1.92 (dd, 1H); 2.05 (t, 1H); 2.43 (s, 6H); 2.62 (t, 1H); 2.98 (m, 1H); 3.35 (t, 1H); 3.75 (t, 2H); 3.84 (d, 1H); 4.67 (m, 1H); 4.81 (dd, 1H); 5.40 (s, 1H); 7.25 (t, 1H); 8.13 (s, 1H).

### Example 45

### 8-(dimethylamino)-3,10a-dimethyl-2,6-dioxodecahydro-4,7-methenofuro[3,2-c]oxireno[f]oxacycloundecin-9-yl thien-3-ylacetate

This compound is obtained by a procedure similar to that described for the synthesis of the compound of Example 44. The expected product is obtained in the form of a white powder.

NMR-¹H (DMSO): 1.14 (s, 3H); 1.26 (d, 3H); 1.94 (dd, 1H); 2.05 (m, 1H); 2.37 (s, 6H); 2.61 (t, 1H); 2.97 (m, 1H); 3.33 (t, 1H); 3.77 (s, 2H); 3.88 (d, 1H); 4.67 (m, 1H); 4.78 (dd, 1H); 5.39 (s, 1H); 7.04 (d, 1H); 7.36 (s, 1H); 7.50 (t, 1H); 8.11 (s, 1H).

### Example 46

### 8-(dimethylamino)-3,10a-dimethyl-2,6-dioxodecahydro-4,7-methenofuro[3,2-c]oxireno[f]oxacycloundecin-9-yl 1-benzothien-3-ylacetate

This compound is obtained by a procedure similar to that described for the synthesis of the compound of Example 44. The expected product is obtained in the form of a white powder.

NMR-¹H (DMSO): 1.13 (s, 3H); 1.25 (d, 3H); 1.94 (dd, 1H); 2.04 (t, 1H); 2.26 (s, 6H); 2.60 (t, 1H); 2.96 (m, 1H); 3.32 (m, 1H); 3.88 (d, 1H); 4.04 (s, 2H); 4.67 (m, 1H); 4.74 (dd, 1H); 5.38 (s, 1H); 7.40 (m, 2H); 7.64 (s, 1H); 7.79 (d, 1H); 7.99 (d, 1H); 8.09 (s, 1H).

### Example 47

### 8-(dimethylamino)-3,10a-dimethyl-2,6-dioxodecahydro-4,7-methenofuro[3,2-c]oxireno[f]oxacycloundecin-9-yl thiophene-3-carboxylate

This compound is obtained by a procedure similar to that described for the synthesis of the compound of Example 44. The expected product is obtained in the form of a white powder.

NMR-¹H (DMSO): 1.19 (s, 3H); 1.27 (d, 3H); 1.97 (dd, 1H); 2.07 (t, 1H); 2.48 (s, 6H); 2.65 (t, 1H); 2.99 (m, 1H); 3.46 (d, 1H); 3.98 (s, 1H); 4.70 (m, 1H); 5.02 (m, 1H); 5.43 (s, 1H); 7.49 (s, 1H); 7.69 (s, 1H); 8.18 (s, 1H); 8.40 (s, 1H).

### Example 48

### 8-(dimethylamino)-3,10a-dimethyl-2,6-dioxodecahydro-4,7-methenofuro[3,2-c]oxireno[f]oxacycloundecin-9-yl 5-phenylthien-2-ylcarbamate

This compound is obtained by a procedure similar to that described for the synthesis of the compound of Example 37. The expected product is obtained in the form of a white powder.

NMR-¹H (DMSO): 1.20 (s, 3H); 1.27 (d, 3H); 1.97 (dd, 1H); 2.03 (t, 1H); 2.38 (s, 6H); 2.67 (m, 1H); 2.98 (m, 1H); 3.37 (d, 1H); 3.94 (m, 1H); 4.69 (m, 1H); 4.81 (m, 1H); 5.41 (s, 1H); 6.60 (s, 1H); 7.22 (m, 2H); 7.36 (t, 2H); 7.55 (d, 2H); 8.14 (s, 1H); 10.93 (s, 1H).

### Example 49

### 8-(dimethylamino)-3,10a-dimethyl-2,6dioxodecahydro-4,7-methenofuro[3,2-c]oxireno[f]oxacycloundecin-9-yl 1-adamantylcarbamate

This compound is obtained by a procedure similar to that described for the synthesis of the compound of Example 37. The expected product is obtained in the form of a white powder.

NMR-¹H (DMSO): 1.14 (s, 3H); 1.26 (d, 3H); 1.60 (s, 6H); 1.80-1.94 (m, 6H); 1,94-2.09 (m, 4H); 2.47 (s, 6H); 2.62 (t, 1H); 2.96 (m, 1H); 3.21 (d, 1H); 3.38 (s, 1H); 3.76 (s, 1H); 4.64 (m, 2H); 5.37 (s, 1H); 6.96 (s, 1H); 8.05 (s, 1H).

### Example 50

### 8-dimethylamino)-3,10a-dimethyl-2,6dioxodecahydro-4,7-methenofuro[3,2-c]oxireno[f]oxacycloundecin-9-yl 2-naphthylcarbamate

This compound is obtained by a procedure similar to that described for the synthesis of the compound of Example 37. The expected product is obtained in the form of a white powder.

NMR-¹H (DMSO): 1.20 (s, 3H); 1.28 (d, 3H); 1.96 (dd, 1H); 2.07 (t, 1H); 2.48 (s, 6H); 2.66 (t, 1H); 3.01 (m, 1H); 3.37 (m, 1H); 3.95 (d, 1H); 4.70 (m, 1H); 4.87 (dd, 1H); 5.42 (s, 1H); 7.38 (t, 1H); 7.46 (t, 1H); 7.55 (d, 1H); 7.82 (m, 3H); 8.10 (s, 1H); 8.14 (s, 1H); 10.01 (s, 1H).

### Example 51

### 8-(dimethylamino)-3,10a-dimethyl-2,6-dioxodecahydro-4,7-methenofuro[3,2-c]oxireno[f]oxacycloundecin-9-yl2-tert-butyl-6-methylphenylcarbamate

This compound is obtained by a procedure similar to that described for the synthesis of the compound of Example 37. The expected product is obtained in the form of a white powder.

NMR-¹H (DMSO): 1.14 (s, 3H); 1,20-1.42 (m, 12H); 1.92 (dd, 1H); 2.05 (m, 1H); 2.25 (s, 3H); 2.52 (s, 6H); 2.62 (m, 1H); 2.95 (m, 1H); 3.36 (m, 1H); 3.88 (m, 1H); 4.80-4.95 (m, 2H); 5.40 (s, 1H); 7.13 (m, 2H); 7.22 (s, 1H); 8.13 (s, 1H); 8.69 (s, 1H).

### Example 52

### 8-(dimethylamino)-3,10a-dimethyl-2,6-dioxodecahydro-4,7-methenofuro[3,2-c]oxireno[f]oxa-cycloundecin-9-yl 2,5-dimethoxyphenylcarbamate

This compound is obtained by a procedure similar to that described for the synthesis of the compound of Example 37. The expected product is obtained in the form of a white powder.

NMR-¹H (DMSO): 1.17 (s, 3H); 1.27 (d, 3H); 1.94 (dd, 1H); 2.06 (m, 1H); 2.48 (s, 6H); 2.64 (t, 1H); 2.98 (m, 1H); 3.33 (m, 1H); 3.69 (s, 3H); 3.76 (s, 3H); 3.89 (d, 1H); 4.68 (m, 1H); 4.80 (dd, 1H); 5.40 (s, 1H); 6.63 (d, 1H); 6.94 (d, 1H); 7.32 (s, 1H); 8.12 (s, 1H); 8.58 (s, 1H).

### β) Examples 53 to 69

The compounds of Examples 53 to 69 are compounds of general formula (III). These compounds were characterised by their retention time and their molecular mass peak (MH+) as described hereafter.

The compounds are characterised by their retention time (r.t.), expressed in minutes, determined by liquid chromatography (LC), and their molecular peak (MH⁺) determined by mass spectrometry (MS), a single quadrupole mass spectrometer (Micromass, Platform model) equipped with an electrospray source is used with a resolution of 0.8 da to 50% valley. For Examples 53 to 69 hereafter, the elution conditions corresponding to the results indicated are the following: change of an acetonitrile-water-trifluoroacetic acid mixture 50-950-0.2 (A) to an acetonitrile-water 950-50 mixture (B) by a linear gradient over a period of 8.5 minutes, then elution with pure mixture B for 10.5 minutes.

### Example 53

### 2-methyl-5-{[2-(4-morpholinyl)ethyl]amino}-1,3-benzothiazole-4,7-dione

51.2 μl (0.39 mmol; 3 equivalents) of 4-(2-aminoethyl)morpholine is added to 27 mg (0.129 mmol) of 5-methoxy-2-methyl-4,7-dioxobenzothiazole in solution in 2 ml of anhydrous ethanol. The reaction mixture is stirred under reflux for 18 hours then the solvent is evaporated off under reduced pressure. The residue is purified on a silica column (eluent: methanol at 5% in dichloromethane). The expected compound is obtained in the form of a red powder.

NMR ¹H (DMSO d6, 400 MHz, 8): 7.45 (t, 1H, NH); 5.49 (s, 1H, CH); 3.58-3.55 (m, 4H, 2 CH₂); 3.26 (t, 2H, CH₂); 2.75 (s, 3H, CH₃); 2.54 (t, 2H, CH₂); 2.42-2.40 (m, 4H, 2 CH₂).

MS-LC: MH+=308.25; r.t.=6.89 minutes.

The compounds of the examples 54 to 66 are obtained in a similar way to that used for Example 53.

### Example 54

### 5-{[2-(dimethylamino)ethyl]amino}-2-methyl-1,3-benzothiazole-4,7-dione

NMR ¹H (DMSO d6, 400 MHz, 8): 7.34 (t, 1H, NH); 5.48 (s, 1H, CH); 3.24-3.20 (m, H, CH₂); 2.77 (s, 3H, CH₃); 2.47 (m, 2H, CH₂); 2.18 (s, 6H, 2 CH₃).

MS-LC: MH+=266.27; r.t.=6.83 minutes.

### Example 55

### 5-{[6-(dimethylamino)hexyl]amino}-2-methyl-1,3-benzothiazole-4,7-dione

MS-LC: MH+=322.33; r.t.=7.36 minutes.

### Example 56

### 5-{[3-(dimethylamino)-2,2-dimethylpropyl]amino}-2-methyl-1,3-benzothiazole-4,7-dione

NMR ¹H (DMSO d6, 400 MHz, 8): 8.62 (t, 1H, NH); 5.45 (s, 1H, CH); 3.07-3.06 (m, 2H, CH₂); 2.74 (s, 3H, CH₃); 2.29-2.30 (m, 2H, CH₂); 2.27 (s, 6H, 2CH₃); 0.93 (s, 6H, 2 CH₃).

LC-MS: MH+=308.32; r.t.=7.16 minutes.

### Example 57

### 2-methyl-5-{[3-(4-methyl-1-piperazinyl)propyl]amino}-1,3-benzothiazole-4,7-dione

NMR ¹H (DMSO d6, 400 MHz, 6): 8.14 (t, 1H, NH); 5.46 (s, 1H, CH); 3.25-3.26 (m, 2H, CH₂); 3.21-3.19 (m, 2H, CH₂); 2.74 (s, 3H, CH₃); 2.49-2.48 (m, 2H, CH₂); 2.37-2.32 (m, 6H, 3CH₂); 2.16 (s, 3H, CH₃); 1.72 (t, 2H, CH₂).

MS-LC: MH+=335.34; r.t.=6.87 minutes.

### Example 58

### 5-[(1-ethylhexyl)amino]-2-methyl-1,3-benzothiazole-4,7-dione

MS-LC: MH+=307.32; r.t.=11.45 minutes.

### Example 59

### 5-[(1-adamantylmethyl)amino]-2-methyl-1,3-benzothiazole-4,7-dione

MS-LC: MH+=343.31; r.t.=11.73 minutes.

### Example 60

### 2-methyl-5-[(2-thienylmethyl)amino]-1,3-benzothiazole-4,7-dione

MS-LC: MH+=291.16; r.t.=9.24 minutes.

### Example 61

### 5-[(3-chlorobenzyl)amino]-2-methyl-1,3-benzothiazole-4,7-dione

MS-LC: MH+=319.24; r.t.=9.95 minutes.

### Example 62

### 2-methyl-5-[(4-pyridinylmethyl)amino]-1,3-benzothiazole-4,7-dione

MS-LC: MH+=286.13; r.t.=6.97 minutes.

### Example 63

### 2-methyl-5-(propylamino)-1,3-benzothiazole-4,7-dione

MS-LC: MH+=237.16; r.t.=8.74 minutes.

### Example 64

### 5-{[3-(1H-imidazol-1-yl)propyl]amino}-2-methyl-1,3-benzothiazole-4,7-dione

MS-LC: MH+=303.17; r.t.=7.07 minutes.

### Example 65

### 4-{2-[(2-methyl-4,7-dioxo-4,7-dihydro-1,3-benzothiazol-5-yl)amino]ethyl}benzenesulphonamide

MS-T C: MH+=378.10; r.t.=8.31 minutes.

### Example 66

### 5-(4-benzyl-1-piperazinyl)-2-methyl-1,3-benzothiazole-4,7-dione

MS-LC: MH+=354.19; r.t.=7.53 minutes.

### Example 67

### 5-anilino-2-ethyl-4,7-dihydrobenzo[d][1.3]oxazole-4,7-dione

67.1) 2-ethyl-4-nitrobenzo[d][1.3]oxazole:

A mixture of 2-amino-3-nitrophenol (1 eq.), triethyl orthopropionate (2 eq.) and p-toluenesulphonic acid (in catalytic quantity) is stirred at 110° C. until the aminophenol disappears this being verified by thin layer chromatography (2 hours). After cooling down, the reaction mixture is taken up in toluene and evaporated under vacuum then treated with isopropanol. The resulting precipitate is recovered by filtration, washed with isopropanol and isopentane, then dried under reduced pressure in order to produce a brown-purple solid.

NMR ¹H (DMSO d6, 400 MHz, 8): 8.15 (dd, 2H); 7.58 (t, 1H); 3.06 (q, 2H); 1.38 (t, 3H).

MS-LC: MH+=193.02; r.t.=9.23 minutes.

67.2) 2-ethylbenzo[d][1.3]oxazol-4-amine:

2-ethyl-4-nitrobenzo[d][1.3]oxazole is hydrogenated under a pressure of 8 bars in the presence of palladium carbon at 10% (0.01 eq.) using methanol as solvent. The catalyst is separated by filtration and the methanol is eliminated under reduced pressure. The residue is taken up in ethyl ether in order to produce a pale purple solid which is recovered by filtration and dried. Melting point: 46° C.

NMR ¹H (DMSO d6, 400 MHz, 8): 6.97 (t, 1H); 6.72 (d, 1H); 6.47, d, 1H); 5.45 (s, 2H); 2.87 (q, 2H); 1.32 (t, 3H).

MS-LC: MH+=162.99; r.t.=8.72 minutes.

67.3) 2-ethyl-4,7-dihydrobenzo[d][1.3]oxazole-4,7-dione:

A solution of [bis(trifluoroacetoxy)iodo]benzene (2.2 eq.) in a mixture of acetonitrile and water (80/20) is added dropwise to a solution of 2-ethylbenzo[d][1.3]oxazol-4-amine (1 eq.) in the same acetonitrile/water mixture maintained at −5° C. The reaction medium is then diluted with water and extracted with dichloromethane. The resulting organic phase is washed with water, dried over sodium sulphate and concentrated in order to produce a brown paste. Purification by chromatography at medium-pressure on silica gel produces, after being taken up in diisopropyl ether, a yellow crystalline solid.

Melting point: 99° C.

NMR ¹H (CDCl₃, 400 MHz, 8): 6.75 (dd, 2H); 2.99 (q, 2H); 1.45 (t, 3H).

MS-LC: MH+=177.83; r.t.=8.29 minutes.

67.4) 5-anilino-2-ethyl-4,7-dihydrobenzo[d][1.3]oxazole-4,7-dione:

A mixture of 2-ethyl-4,7-dihydrobenzo[d][1.3]oxazole-4,7-dione (1 eq) and aniline (1.1 eq.) in ethanol is maintained under stirring for 1 hour. The reaction medium turns dark purple. After concentration, the residue is purified by chromatography at medium pressure on silica in order to produce a purple powder. Melting point: 200° C.

NMR ¹H (CDCl₃, 400 MHz, 8): 9.38 (s, 1H); 7.44 (t, 2H); 7.36 (d, 2H); 7.22 (t, 1H); 5.69 (s; 1H); 2.94 (q, 2H); 1.29 (t, 3H).

MS-LC: MH+=269.11; r.t.=9.76 minutes.

### Example 68

### 5-anilino-6-chloro-2-ethyl-4,7-dihydrobenzo[d][1.3]oxazole-4,7-dione

A solution of 5-anilino-2-ethyl-4,7-dihydrobenzo[d][1.3]oxazole-4,7-dione (1 eq.) in acetic acid is treated with N-chlorosuccinimide (1.1 eq.) at ambient temperature. The reaction medium is maintained under stirring for 2 hours before being concentrated, taken up in ethanol and concentrated again. The residue is purified by chromatography at medium pressure on silica in order to produce a purple powder. Melting point: 159° C.

NMR ¹H (CDCl₃, 400 MHz, 8): 9.39 (s, 1H); 7.30 (t, 2H); 7.11 (m, 3H); 2.96 (q, 2H); 1.30 (t, 3H).

MS-LC: MH+=303.01; r.t.=10.28 minutes.

### Example 69

### 2-ethyl-5-(4-fluoroanilino)-4,7-dihydrobenzo[d][1.3]oxazole-4,7-dione

The experiment protocol used is identical to that described for Example 67, 4-fluoroaniline replacing the aniline in the fourth and last stage. Melting point: 232° C.

NMR ¹H (CDCl₃, 400 MHz, 6): 9.38 (s, 1H); 7.37 (t, 2H); 7.26 (t, 2H); 5.57 (s, 1H); 2.93 (q, 2H); 1.30 (t, 3H).

MS-LC: MH+=287.09; r.t.=9.88 minutes.

### γ) Examples 70 to 102

The compounds of Examples 70 to 102 are compounds of general formula (IV).

### Example 70

### 8-bromo-4-[2-(5methyl-4-imidazolylmethylthio)-ethylamino]-2-methylthlopyrazolo[1,5-a]-1,3,5-triazlne

This compound was prepared according to the method described in the American patent 4,565,815. Mass spectrometry (Electrospray): 416.0.

### Example 71

### 8-bromo-4-{2-{[5-(dimethylamino)methyl-2-furannyl]-methyl}thio}ethylamino-2-methylthiopyrazolo[1,5-a]-1,3,5-triazine

This compound was prepared according to the method described in the American patent 4,565,815. Mass spectrometry (Electrospray): 459.1.

### Example 72

### 8-bromo-4-(3-(1-imidazolyl-propylamino)-2-methylthiopyrazolo[1,5-a]-1,3,5-triazine

, 60 μl of 1-(3-aminopropyl)imidazole is added to a solution of 8-bromo-4-chloro-2-methylthiopyrazolo[1,5-a]-1,3,5-triazine (50 mg) in a mixture of 2 ml of chloroform and 2 ml of methanol and the mixture is stirred overnight at ambient temperature. After evaporation of the solvents, the residue is divided between chloroform and water. The organic phase is then dried over MgSO₄, then, after evaporation of the solvents, the residue is subjected to preparatory chromatography on silica gel using a chloroform/methanol mixture 4/1 as eluent. The appropriate fraction is isolated, extracted with a chloroform-methanol mixture and the solvents are evaporated to dryness under vacuum. A white solid is obtained. Thin-layer chromatography (silica gel; chloroform/methanol in a 4/1 mixture): R_{f}=0.32. Mass spectrometry (Electrospray): 368.4; 370.1.

The compounds of Examples 73 to 80 are prepared according to an operating method similar to that of Example 72.

### Example 73

### 8-bromo-4-[(3-pyridyl)methylamino]-2-methylthio-pyrazolo [1,5-a]-1,3,5-triazine

Mass spectrometry (Electrospray): 351.0; 353.0.

### Example 74

### 8-bromo-4-(3-chloroanilino)-2-methylthio-pyrazolo [1,5-a]-1,3,5-triazine

Mass spectrometry (Electrospray): 369.9; 371.9.

### Example 75

### 8-bromo-2-methylthio-4-(4-pyridylmethylamino)pyrazolo[1,5-a]-1,3,5-triazine

Mass spectrometry (Electrospray): 351.0; 352.9.

### Example 76

### 8-bromo-2-methylthio-4-(2-pyridylethylamino)pyrazolo[1,5-a]-1,3,5-triazine

Mass spectrometry (Electrospray): 365.0; 366.9.

### Example 77

### 8-bromo-2-methylthio-4-(2-pyridylmethylamino)pyrazolo[1,5-a]-1,3,5-triazine

A white solid. Mass spectrometry (Electrospray): 351.0; 352.9.

### Example 78

### 8-bromo-2-methylthio-4-(4-fluorophenylmethylamino)-pyrazolo[1,5-a]-1,3,5-triazine

A white solid. Mass spectrometry (Electrospray): 367.9; 369.9.

### Example 79

### 8-bromo-2-methylthio-4-(3-fluorophenylmethylamino)-pyrazolo[1,5-a]-1,3,5-triazine

A white solid. Mass spectrometry (Electrospray): 367.9; 369.8.

### Example 80

### 8-bromo-2-methylthio-4-[4-N-methylpiperazinyl)anilinolpyrazolo[1,5-a]-1,3,5-triazine

A white powder. Melting point: 223-224° C.

### Example 81

### 8-bromo-2-(1R-isopropyl-2-hydroxyethylamino)-4-(3-chloroanilino)-pyrazolo[1,5-a]-1,3,5-triazine

81.1) 8-bromo-4-(3-chloroanilino)-2-methylthioxo-pyrazolo[ ], 5-a]-1,3,5-triazine

280 mg of m-chloroperbenzoic acid is added to a solution of 8-bromo-4-(3-chloroanilino)-2-methylthio-pyrazolo[1,5-a]-1,3,5-triazine (200 mg; prepared in a similar way to that used for the compounds of examples 72 to 74 starting from 8-bromo-4-chloro-2-methylthiopyrazolo[1,5-a]-1,3,5-triazine and some 3-chloroaniline) in 5 ml of chloroform. The mixture is stirred overnight at ambient temperature. The reaction medium is diluted with chloroform (10 ml) and is washed with an aqueous solution of NaHSO₃ then with an aqueous solution of NaHCO₃. The organic phase is dried over MgSO₄ and the solvents are evaporated to dryness under vacuum. 200 mg of a brown solid is obtained. Mass spectrometry (Electrospray): 402.0; 404.0.

81.2) 8-bromo-2-(1R-isopropyl-2-hydroxyethylamino)-4-(3-chloroanilino)-pyrazolo[1,5-a]-1, 3, 5-triazine

2 ml of solution of R-Valinol in propanol (50 mg/ml) is added to a partial suspension of the intermediate 81.1 (130 mg) in 5 ml of chloroform. The resulting mixture is stirred overnight at ambient temperature. After evaporation of the solvents, the residue is subjected to preparatory chromatography on silica gel using a chloroform/acetone mixture (9:1) as eluent. The appropriate fraction is isolated, extracted with a chloroform-acetone mixture and the solvents are evaporated to dryness under vacuum. A brown solid is obtained. TLC (silica gel; chloroform/acetone in a 9/1 mixture): R_{f}=0.28. Mass spectrometry (Electrospray): 425.1; 427.0.

The compounds of Examples 82 to 86 are prepared according to an operating method similar to that of Example 81.

### Example 82

### 8-bromo-2-(2-aminocyclohexylamino)₄-(3-chloroanilino)-pyrazolo[1,5-a]-1,3,5-triazine

A pale yellow solid. Mass spectrometry (Electrospray): 436.1; 438.1.

### Example 83

### 8-bromo-2-(1R-isopropyl-2-hydroxyethylamino)₄-(3-oxido-pyridylmethylamino)-pyrazolo[1,5-a]-1,3,5-triazine

A pale yellow-brown liquid. Mass spectrometry=422.1.

### Example 84

### 8-bromo-2-(1R-isopropyl-2-hydroxyethylamino)-4-(3-fluorophenylmethylamino)-pyrazolo[1,5-a]-1,3,5-triazine

Mass spectrometry (Electrospray): 424.9.

### Example 85

### 8-bromo-2-(4′-hydroxyethylpiperazinyl)₄-(3-oxido-pyridylmethylamino)-pyrazolo[1,5-a]-1,3,5-triazine

Mass spectrometry (Electrospray): 451.0.

### Example 86

### 8-bromo-2-(4′-hydroxyethylpiperazinyl)₄-(3-pyridylmethylamino)-pyrazolo[1,5-a]-1,3,5-triazine

Mass spectrometry (Electrospray): 435.0.

### Example 87

### 2,4-bis-(3-pyridylmethylamino)-8-bromo-pyrazolo[1,5-a]-1,3,5-triazine

430 mg of m-chloroperbenzoic acid is added to a solution of 8-bromo-4-chloro-2-methylthio-pyrazolo[1,5-a]-1,3,5-triazine (270 mg) in 10 ml of chloroform. The mixture is stirred for one hour at ambient temperature. 4 equivalents of 3-aminomethylpyridine are added and the mixture is stirred overnight at ambient temperature. After dilution with chloroform (20 ml) and washing with water, the recovered organic phase is dried over MgSO₄. After evaporation of the solvents, the residue is subjected to preparatory chromatography on silica gel using a chloroform/methanol mixture 95/5 as eluent. The appropriate fraction is isolated, extracted with a chloroform-methanol mixture and the solvents are evaporated to dryness under vacuum. A yellow solid is obtained. TLC (silica gel; chloroform/methanol in a 9/1 mixture): R_{f}=0.33. Mass spectrometry (Electrospray): 411.2; 413.2.

### Example 88

### 2,4-bis-(2-pyridylmethylamino)-8-bromo-pyrazolo[1,5-a]-1,3,5-triazine

This compound is prepared according to a operating method similar to that described for Example 86. A yellow solid. Mass spectrometry (Electrospray): 383.1; 385.1.

### Example 89

### 8-acetyl-4-(3-pyridylmethylamino)-2-methylthiopyrazolo[1,5-a]-1,3,5-triazine

213 mg AlCl₃ then 90 μl of acetyl chloride are added successively to a solution of 2-methylthio-4-(3-pyridylmethylamino)-pyrazolo-[1,5-a]-1,3,5-triazine (110 mg) in 15 ml of dichloromethane. The mixture is taken to reflux for 4 hours. After diluting with chloroform (20 ml), the mixture is acidified with dilute HCl, then basified with an aqueous solution of NaHCO₃ and the recovered organic phase is dried over MgSO₄. The solvents are eliminated by evaporation to dryness under vacuum. The residue is subjected to preparatory chromatography on silica gel using a chloroform/acetone mixture (9:1) as eluent. The appropriate portions are isolated, extracted with a chloroform-methanol mixture and the solvents are eliminated by evaporation to dryness under vacuum. 65 mg of a white solid is obtained. TLC (silica gel; chloroform/acetone in a 9/1 mixture): R_{f}=0.18. Mass spectrometry (Electrospray): 315.1.

### Example 90

### 8-dimethylaminomethyl-4-(3-pyridylmethylamino)-2-methylthiopyrazolo[1,5-a]-1,3,5-triazine

A solution of 2-methylthio-4-(3-pyridylmethylamino)-pyrazolo[1,5-a]-1,3,5-triazine (50 mg) and (chloromethylene)-dimethylammonium chloride (2 equivalents) in a mixture of acetonitrile and dimethylformamide (4:1; 10 ml) is taken to reflux for 4 hours. The solvents are eliminated by evaporation to dryness under vacuum. The residue is dissolved in 20 ml of ethanol and treated with an excess of NaBH₄. After stirring for 2 hours at ambient temperature, acetic acid is added to the reaction mixture to break down the excess reagent. After eliminating the solvents under vacuum, the residue is divided between CHCl₃ and water. The recovered organic phase is dried over MgSO₄. After elimination of the solvents, the residue is subjected to preparatory chromatography on silica gel using a chloroform-methanol mixture (3:1) as eluent. The appropriate portions are isolated and extracted with a chloroform-methanol mixture and the solvents are eliminated by evaporation to dryness under vacuum. 19 mg of an ochre powder is obtained. TLC (silica gel; chloroform/methanol in a 3/1 mixture): R_{f}=0.19.

Mass spectrometry (Electrospray): 330.1.

### Example 91

### 8-formyl-4-(3-pyridylmethylamino)-2-methylthiopyrazolo[1,5-a]-1,3,5-triazine

2-methylthio-4-(3-pyridylmethylamino)-pyrazolo[1,5-a]-1,3,5-triazine (100 mg) and (chloromethylene)-dimethylammonium chloride (4 equivalents) in an acetonitrile-dimethylformamide mixture (4:1; 50 ml) are taken to reflux for 2 hours. After evaporation of the solvents, the residue is dissolved in tetrahydrofuran (50 ml) and 25 ml of a 0.5M aqueous solution of sodium acetate. After stirring for 4 hours at ambient temperature, the larger part of the tetrahydrofuran is eliminated under vacuum. The concentrated residue is divided between chloroform and water. The recovered organic phase is then dried over MgSO₄ and the solvents are evaporated under vacuum in order to produce 8-formyl-2-methylthio-4-(3-pyridylmethylamino)-pyrazolo[1,5-a]-1,3,5-triazine. TLC (silica gel; chloroform/methanol mixture=9/1): R_{f}=0.5. Mass spectrometry (Electrospray): 301.0.

### Example 92

### 8-morpholinomethyl-4-(3-pyridylmethylamino)-2-methylthiopyrazolo[1,5-a]-1,3,5-triazine

3 Å molecular sieves (0.5 g) and Na(OAc)₃BH (134 mg) are added to a solution of 8-formyl-4-(3-pyridylmethylamino)-2-methylthiopyrazolo[1,5-a]-1,3,5-triazine (90 mg) and morpholine (52 mg) in 40 ml of dichloroethylene containing 1% acetic acid. The mixture obtained is stirred overnight at ambient temperature. The reaction mixture is filtered and the filtrate diluted with chloroform (50 ml). The resulting solution is then washed with an aqueous solution of NaHCO₃ and an aqueous solution of NaCl before being dried over MgSO₄. After evaporation of the solvents, the residue is subjected to preparatory chromatography on silica gel using a chloroform/methanol mixture (9:1) as eluent. The appropriate portions are isolated and extracted with a chloroform-methanol mixture and the solvents are eliminated by evaporation to dryness under vacuum. 26 mg of an whitish solid is obtained. TLC (silica gel; chloroform/methanol mixture=9/1): R_{f}=0.19. Mass spectrometry (Electrospray): 372.2.

### Example 93

### 8-[(1,3-dihydro-2-oxoindol)-3-ylidenemethyl)-2-methylthio-4-(3-pyridylmethylamino)pyrazolo[1,5-a]-1,3,5-triazine

A mixture of 8-formyl-2-methylthio-4-(3-pyridylmethylamino)-pyrazolo[1,5-a]-1,3,5-triazine (70 mg), oxoindole (64 mg) and a drop of piperidine in 50 ml of ethanol is taken to reflux for 7 hours. After returning to ambient temperature, a yellow solid is recovered by filtration and dried. TLC (silica gel; chloroform/methanol mixture=9/1: R_{f}=0.49). Mass spectrometry (Electrospray): 416.2.

### Example 94

### 8-(guanidinoaminomethylene)-2-methylthio-4-(3-pyridylmethylamino)pyrazolo[1,5-a]-1,3,5-triazine

This compound is prepared according to an operating method similar to that described for Example 93, the oxoindole being replaced by aminoguanidine bicarbonate. A brown solid. Mass spectrometry (Electrospray): 359.2.

### Example 95

### 8-bromo-2-methylthioxo-4-(3-pyridylmethylamino)pyrazolo[1,5-a]-1,3,5-triazine

This compound is prepared according to an operating method similar to that described for intermediate 81.1. Dark yellow powder. Melting point: 70-71° C.

### Example 96

### 8-bromo-2-methylthioxo-4-(3-chloroanilino)pyrazolo[1,5-a]-1,3,5-triazine

It is intermediate 81.1.

### Example 97

### 8-[(1,3-dihydro-2-oxoindol)-3-ylidenemethyl]-2-methylthio-4-[3-(1-imidazolyl)propylaminolpyrazolo[1,5-a]-1,3,5-triazine

This compound is prepared according to an operating method similar to that described for Example 93, 8-formyl-2-methylthio-4-(3-pyridylmethylamino)pyrazolo[1,5-a]-1,3,5-triazine being replaced by 8-formyl-2-methylthio-4-(3-(1-imidazolyl)propylamino)pyrazolo[1,5-a]-1,3,5-triazine. A yellow solid. Mass spectrometry (Electrospray): 433.2.

### Example 98

### 8-cyano-2-methylthio-4-(3-pyridylmethylamino)pyrazolo[1,5-a]-1,3,5-triazine

This compound is prepared by heating to reflux a mixture containing the compound of Example 91 (1 equivalent), hydroxylamine hydrochloride (2 equivalents), sodium formate (10 equivalents) and formic acid (100 equivalents) (cf. J. Chem. Soc. (1965), 1564). A pale yellow solid. Mass spectrometry (Electrospray): 298.2.

### Example 99

### 8-(N-methylpiperazinomethyl)-2-methylthio-4-(3-pyridylmethylamino)pyrazolo[1,5-a]-1,3,5-triazine

This compound is prepared according to an operating method similar to that described for Example 92, morpholine being replaced by N-methylpiperazine. A brown solid.

Mass spectrometry (Electrospray): 385.4; 386.4.

### Example 100

### 2-methylthio-4-(3-pyridylmethylamino)pyrazolo[1,5-a]-1,3,5-triazine

3-aminomethylpyridine (3.0 g) is added to a solution of 4-chloro-2-methylthiopyrazolo[1,5-a]-1,3,5-triazine (2.0 g) in 40 ml of chloroform and 14 ml of methanol. The mixture obtained is stirred overnight at ambient temperature. After evaporation of the solvents to dryness under vacuum, the residue is divided between chloroform and water. The organic phase is dried over MgSO₄ and the solvents are evaporated to dryness under vacuum. The residual mixture is subjected to chromatography on silica gel using a chloroform/methanol mixture (19:1) as eluent. The appropriate portions are isolated and the solvents are eliminated by evaporation to dryness under vacuum. 1.47g of a white solid is obtained. TLC (silica gel; chloroform/methanol mixture=19/1): R_{f}=0.58. Mass spectrometry (Electrospray): 273.1.

### Example 101

### 2-methylthio-8-nitro-4-(3-pyridylmethylamino)pyrazolo[1,5-a]-1,3,5-triazine

Cupric nitrate (70 mg) is added to a suspension of 2-methylthio-4-(3-pyridylmethylamino)-pyrazolo[1,5-a]-1,3,5-triazine (50 mg; compound of Example 100) in 6 ml of acetic anhydride. The mixture is stirred at ambient temperature overnight before being divided between chloroform and a saturated aqueous solution of NaHCO₃. The organic phase is dried over MgSO₄ and the solvents are evaporated to dryness under vacuum. The residue is subjected to preparatory chromatography on silica gel using a chloroform-methanol mixture (15:1) as eluent. The appropriate fraction is isolated and extracted with a chloroform methanol mixture. Once the solvents are evaporated to dryness under vacuum, the expected product is obtained in the form of a whitish solid. Thin layer chromatography (silica gel; chloroform-methanol mixture 9:1): R_{f}=0.46. Mass spectrometry (Electrospray): 318.1.

### Example 102

### 8-bromo-2-(1R-isopropyl-2-hydroxyethylamino)-4-(3-pyridylmethylamino)pyrazolo[1,5-a]-1,3,5-triazine

102.1) 8-bromo-2-methylthioxo-4-(3-pyridylmethylamino)-pyrazolo[1,5-a]-1,3,5-triazine

100 mg of oxone is added to a solution of 8-bromo-2-methylthio-4-(3-pyridylmethylamino)-pyrazolo[1,5-a]-1,3,5-triazine hydrochloride (100 mg) in an ethanol-water mixture (1:1; 50 ml). After 15 minutes, the mixture is diluted with water (20 ml), NaHCO₃ is added in order to render the medium basic and extraction is carried out with a chloroform-methanol mixture (9:1). The organic phase is dried (MgSO₄) and the solvents are eliminated in order to produce the expected product in the form of a pale yellow solid (100 mg). Mass spectrometry (Electrospray): 367.2; 369.2.

102.2) 8-bromo-2-(]R-isopropyl-2-hydroxyethylamino)-4-(3-pyridylmethylamino)-pyrazolo[1,5-a]-1, 3, 5-triazine

A mixture of the intermediate 33.1 (100 mg) and R-valinol (2 eq.; 60 mg) in 3 ml of CH₃CN is taken to reflux for 3 hours. After evaporation of the solvents, the residue is taken up in a chloroform-methanol mixture (9:1; 30 ml), washed with a saturated aqueous solution of NaCl then dried on MgSO₄. The solvents are eliminated by evaporation to dryness under vacuum and the residue is subjected to preparatory chromatography on silica gel using a chloroform-methanol mixture (19:1) as eluent. The appropriate fraction is isolated and extracted using a chloroform-methanol mixture. The solvents are eliminated by evaporation to dryness under vacuum. The expected product is obtained in the form of a whitish amorphous solid (50 mg). Thin-layer chromatography (silica gel; chloroform-methanol mixture 9:1): R_{f}=0.32. Mass spectrometry (Electrospray): 406.2; 408.2.

Pharmacological Studies

In order to illustrate the usefulness of the invention, the effect of a treatment on a tumorous line of human colic cells HT29 with dihydromikanolide (A₁) or the hydrochloride of 8-(dimethylamino)-3,10a-dimethyl-2,6-dioxodecahydro-4,7-methenofuro[3,2-c]oxireno[f]oxacycloundecin-9-yl 2-naphthylcarbamate (A₂) will be studied in combination with the following anticancer agents:

cisplatin (compound B₁);

7-(2-amino-1-oxo-3-thiopropyl)-8-(cyclohexylmethyl)-2-phenyl-5,6,7,8-tetrahydroimidazo[1,2a]pyrazine (compound B₂);

8-bromo-2-(1R-isopropyl-2-hydroxyethylamino)₄-(3-pyridylmethylamino)-pyrazolo[1,5-a]-1,3,5-triazine (compound B₃);

4-(2-bromophenyl)-1-(2-(1-((4-cyano-3-methoxy)phenylmethyl)imidazo-5-yl)-1-oxoethyl)-1,2-dihydro-8-fluoroimidazol[1,2a][1,4]-benzodiazepine (compound B₄); and

5-{[2-(dimethylamino)ethyl]amino}-2-methyl-1,3-benzothiazole-4,7-dione hydrochloride (compound B₅).

1) Procedures

Cell Line

The cell line HT-29 (human colon cancer cells) was acquired from the American Tissue Culture Collection (Rockville, Md., USA).

Measurement of Cellular Proliferation In Vitro

The HT-29 cells (4000 cells/wells) are cultured on 96-well plates.

On day 0, these cells are seeded in 90 μl of Dulbecco's modified Eagle medium (Gibco-Brl, Cergy-Pontoise, France) completed with 10% of foetal calf serum inactivated by heating (Gibco-Brl, Cergy-Pontoise, France), 50000 units/l of penicillin and 50 mg/l of streptomycin (Gibco-Brl, Cergy-Pontoise, France), and 2 mM of glutamine (Gibco-Brl, Cergy-Pontoise, France).

The cells were treated simultaneously with concentrations of the compounds to be tested singly or in combination for 120 hours.

At the end of the of this period, quantification of the cellular proliferation is evaluated by colorimetric test based on the cleavage of the tetrazolium salt WST1 by the mitochondrial hydrogenases in the living cells leading to the formation of formazan (Boehringer Mannheim, Meylan, France). These tests are carried out four times with 6 determinations for each single product and for each combination tested. This allows determination of the number of living cells at the end of each treatment.

2) Results:

The results obtained for the combinations tested are reported in the Tables I to VI which figure below.

The results reported in the tables, show that the products comprising dihydromikanolide in combination with compound B, or compound B₂ are capable of inhibiting the proliferation in vitro of human tumorous cells HT29 more significantly than the separate products. It is the same for the analogue of dihydromikanolide tested with compounds B₂, B₃, B₄ and B₅.

TABLE I

| | A₁ (5 μg/ml) | B₁ (6.25 μM) | A₁ (5 μg/ml) + B₁ (6.25 μM) |
|---|---|---|---|
| % of living | 61 | 48 | 22 |
| cells | | | |

TABLE II

| | A₁ (5 μg/ml) | B₂ (25 μM) | A₁ (5 μg/ml) + B₂ (25 μM) |
|---|---|---|---|
| % of | 64 | 37 | 13 |
| living cells | | | |

TABLE III

| | A₂ (5 μM) | B₂ (25 μM) | A₂ (5 μM) + B₂ (25 μM) |
|---|---|---|---|
| % of living cells | 67 | 64 | 16.9 |

TABLE IV

| | A₂ (5 μM) | B₃ (100 nM) | A₂ (5 μM) + B₃ (100 nM) |
|---|---|---|---|
| % of living cells | 72 | 104 | 34 |

TABLE V

| | A₂ (5 μM) | B₄ (5 μM) | A₂ (5 μM) + B₄ (5 μM) |
|---|---|---|---|
| % of living cells | 55 | 93 | 33 |

TABLE VI

| | A₂ (5 μM) | B₄ (5 μM) | A₂ (5 μM) + B₄ (5 μM) |
|---|---|---|---|
| % of living cells | 55 | 78 | 8 |

1. Product comprising at least mikanolide, dihydromikanolide or their analogue, optionally in the form of a pharmaceutically acceptable salt, in combination with at least one other anticancer agent for a therapeutic use which is simultaneous, separate or spread over time, in the treatment of cancer.

2. Product according to claim 1, characterized in that the anticancer agent combined with mikanolide, with dihydromikanolide or with their analogue has a different action mechanism to that of said mikanolide, dihydromikanolide or analogue.

3. Product according to claim 1, characterized in that the other anticancer agent is chosen from enzymatic inhibitors, apoptosis inducers, alkylating agents, anti-metabolic agents, differentiation agents, cell spindle poisons, angiogenesis inhibitors, anti-hormones or antagonists of steroid receptors, antioxidants, antisense agents, anti-p53 agents, chemo-prevention agents, antibiotic or anti-viral agents, immuno-therapeutic agents and antibodies.

4. Product according to claim 3, characterized in that the other anticancer agent is chosen from enzymatic inhibitors and alkylating agents.

5. Product according to claim 4, characterized in that the other anticancer agent is an enzymatic inhibitor chosen from topoisomerase inhibitors.

6. Product according to claim 5, characterized in that the topoisomerase inhibitor is chosen from camptothecin analogues.

7. Product according to claim 6, characterized in that the camptothecin analogue is chosen from the following compounds:
(5R)-5-ethyl-9,10-difluoro-5-hydroxy-4,5,13,15-tetrahydro-1H,3H-oxepino[3′,4′:6,7]indolizino[1,2-b]quinoline-3,15-dione;
(5R)-1-[9-chloro-5-ethyl-5-hydroxy-10-methyl-3,15-dioxo-4,5,13,15-tetrahydro-1H,3H-oxepino[3′,4′:6,7]indolizino[1,2-b]quinolin-12-ylmethyl]-4-methyl-hexahydropyridine;
and the pharmaceutically acceptable salts of the latter.

8. Product according to claim 7, characterized in that the camptothecin analogue is (5R)-5-ethyl-9,10-difluoro-5-hydroxy-4,5,13,15-tetrahydro-1H,3H-oxepino[3′,4′:6,7]indolizino[1,2-b]quinoline-3,15-dione or one of its pharmaceutically acceptable salts.

9. Product according to claim 7, characterized in that the camptothecin analogue is (5R)-1-[9-chloro-5-ethyl-5-hydroxy-10-methyl-3,15-dioxo-4,5,13,15-tetrahydro-1H,3H-oxepino[3′,4′:6,7]indolizino[1,2-b]quinolin-12-ylmethyl]-4-methyl-hexahydropyridine.

10. Product according to claim 4, characterized in that the enzymatic inhibitor is a heterotrimeric G protein transduction inhibitor corresponding to general formula (II) 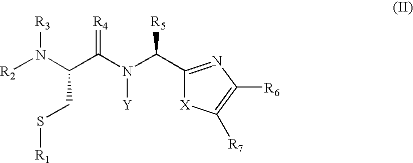
corresponding to sub-formulae (II), or (11)₂: 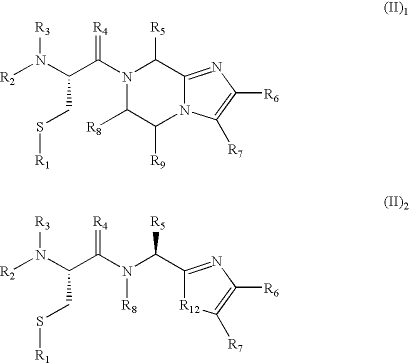
in which:
X represents R₁₂ and Y represents R₈, or X and Y complete a ring with 6 members, the X-Y assembly representing the —CH(R₈)—CH(R₉)— radical;
R₁ represents H, an alkyl or lower alkylthio radical; R₂ and R₃ independently represent H or a lower alkyl radical;
R₄ represents H₂ or 0;
R₅ represents H, or one of the following radicals: lower alkyl, lower cycloalkylalkyl, lower alkenyl, lower alkynyl, aryl, lower arylalkyl, heterocycle or lower alkyl heterocycle, these radicals can optionally be substituted by radicals chosen from the group comprising a lower alkyl, —O—R₁₀, —S(O)ₘR₁₀ (m representing 0, 1, or 2), —N(R₁₀)(R₁₁), —N—C(O)—R₁₀, —NH—(SO₂)—R₁₀, —CO₂—R₁₀, C(O)—N(R₁₀)(R₁ i), and —(SO₂)—N(R₁₀)(R₁₁) radical;
R₆ and R₇ independently represent H, a —C(O)—NH—CHR₁₃—CO₂R₁₄ radical, or one of the following radicals: lower alkyl, aryl, lower arylalkyl, heterocycle or lower alkyl heterocycle, these radicals being optionally substituted by radicals chosen from the group comprising the OH, alkyl or lower alkoxy, N(R₁₀)(R₁₁), COOH, CON(R₁₀)(R₁₁), and halo radicals,
or R₆ and R₇ together form an aryl radical or a heterocycle;
R₈ and R₉ independently represent H, or one of the following radicals: lower alkyl, aryl, lower arylalkyl, heterocycle or lower alkyl heterocycle, these radicals can optionally be substituted by radicals chosen from the group comprising the OH, alkyl or lower alkoxy, N(R₁₀)(R₁₁), COOH, CON(R₁₀)(R₁₁) and halo radicals,
or R₈ and R₉ together form an aryl radical or a heterocycle;
R₁₀ and R₁₁ independently represent H, an aryl radical or heterocycle, or an alkyl, arylalkyl or lower alkyl heterocycle radical;
R₁₂ represents NR₉, S, or O;
R₁₃ represents a lower alkyl radical optionally substituted by a radical chosen from the lower alkyl, —OR₁₀, —S(O)ₘR₁₀ (m representing 0, 1, or 2) and —N(R₁₀)(R₁₁) radicals;
R₁₄ represents H or a lower alkyl radical;
or is a pharmaceutically acceptable salt of said compound of general formula (II).

11. Product according to claim 10, characterized in that the transduction inhibitor of heterotrimeric G protein transduction inhibitor is chosen from:
7-(2-amino-1-oxo-3-thiopropyl)-8-(cyclohexylmethyl)-2-(2-methylphenyl)-5,6,7,8-tetrahydroimidazo[1,2a]pyrazine;
7-(2-amino-1-oxo-3-thiopropyl)-8-(cyclohexylmethyl)-2-phenyl-5,6,7,8-tetrahydroimidazo[1,2a]pyrazine;
7-(2-amino-1-oxo-3-thiopropyl)-2-(2-methoxyphenyl)-8-(phenylmethoxy)methyl-5,6,7,8-tetrahydroimidazo[1,2a]pyrazine;
7-(2-amino-1-oxo-3-thiopropyl)-2-(2-methoxyphenyl)-8-(1-phenylmethoxy)ethyl-5,6,7,8-tetrahydroimidazo[1,2a]pyrazine;
7-(2-amino-1-oxo-3-thiopropyl)-2-(2-methoxyphenyl)-8-(phenoxyethyl)-5,6,7,8-tetrahydroimidazo[1,2a]pyrazine;
7-(2-amino-1-oxo-3-thiopropyl)-2-(2-methoxyphenyl)-8-(phenoxyethyl)-5,6,7,8-tetrahydro-imidazo[1,2a]pyrazine, or its dimeric form;
7-(2-amino-1-oxo-3-thiopropyl)-2-(2-methoxyphenyl)-8-(phenylsulphonylethyl)-5,6,7,8-tetrahydro-imidazo[1,2a]pyrazine;
and their pharmaceutically acceptable salts.

12. Product according to claim 4, characterized in that the other anticancer agent is a Cdc25 phosphatase inhibitor of general formula (III) 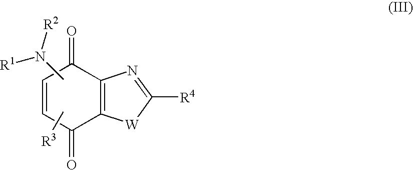
in the racemic form, enantiomeric form or in any combination of these forms, in which:
R¹ represents a hydrogen atom or an alkyl, cycloalkyl, —(CH₂)—X-Y or —(CH₂)-Z-NR⁵R⁶ radical,
R¹ can also, when W represents 0, represent a carbocyclic aryl radical optionally substituted from 1 to 3 times by substituents chosen independently from a halogen atom and an alkyl, haloalkyl or alkoxy radical,
X representing a bond or a linear or branched alkaline radical containing 1 to 5 carbon atoms,
Y representing a saturated carbonated cyclic system containing 1 to 3 condensed rings chosen independently from rings with 3 to 7 members, or Y representing a saturated heterocycle containing 1 to 2 heteroatoms chosen independently from O, N and S and attached to the X radical by a member N or CH, said saturated heterocycle containing moreover from 2 to 6 additional members chosen independently from —CHR⁷—, —CO—, —NR⁸—, —O— and —S—, R⁷ representing a hydrogen atom or an alkyl radical and R⁸ representing a hydrogen atom or an alkyl or aralkyl radical, or also Y representing a carbocyclic or heterocyclic aryl radical optionally substituted from 1 to 3 times by substituents chosen independently from the group constituted by a halogen atom, an alkyl radical, a haloalkyl radical, an alkoxy radical, a haloalkoxy radical, a hydroxy radical, a nitro radical, a cyano radical, the phenyl radical, an SO₂NHR⁹ radical and an NR¹⁰R¹¹ radical, R⁹ representing a hydrogen atom or an alkyl or phenyl radical, and R₁₀ and R¹¹ representing independently alkyl radicals,
Z representing a bond or a linear or branched alkylene radical containing 1 to 5 carbon atoms,
R⁵ and R⁶ being chosen independently from a hydrogen atom, an alkyl, aralkyl or —(CH₂).—OH radical in which n represents an integer from 1 to 6, or R⁵ and R⁶ forming together with the nitrogen atom a heterocycle with 4 to 7 members comprising 1 to 2 heteroatoms, the members necessary to complete the heterocycle being chosen independently from the —CR¹²R¹³—, —O—, —S— and —NR₁₄— radicals, R¹² and R¹³ representing independently each time that they occur a hydrogen atom or an alkyl radical, and R₁⁴ representing a hydrogen atom or an alkyl or aralkyl radical, or also R¹⁴ representing a phenyl radical optionally substituted from 1 to 3 times by substituents chosen independently from a halogen atom and an alkyl or alkoxy radical,
R₂ representing a hydrogen atom or an alkyl radical;
or also R₁ and R² forming together with the nitrogen atom a heterocycle with 4 to 7 members containing 1 to 2 heteroatoms, the members necessary to complete the heterocycle being chosen independently from the radicals —CR¹⁵R¹⁶—, —O—, —S— and —NR₁₇—, R¹⁵ and R 6 representing independently each time that they occur a hydrogen atom or an alkyl radical, and R¹⁷ representing a hydrogen atom or an alkyl or aralkyl radical;
R³ represents a hydrogen atom, a halogen atom, or an alkyl, haloalkyl or alkoxy radical;
R⁴ represents an alkyl, cycloalkyl, cycloalkylalkyl, cyano, amino, —CH₂—COOR¹⁸, —CH₂—CO—NR₁₉R₂₀ or —CH₂—NR²¹R²² radical, or also R⁴ represents a heterocyclic aryl radical optionally substituted from 1 to 3 times by substituents chosen independently from a halogen atom and an alkyl, haloalkyl or alkoxy radical,
R¹⁸ representing a hydrogen atom or an alkyl radical,
R¹⁹ representing a hydrogen atom, an alkyl radical or an aralkyl radical the aryl group of which is optionally substituted from 1 to 3 times by substituents chosen independently from the group constituted by a halogen atom, an alkyl radical, a haloalkyl radical, an alkoxy radical, a haloalkoxy radical, a hydroxy radical, a nitro radical, a cyano radical, the phenyl radical, an SO₂NHR₂₃ radical and an NR²⁴R²⁵ radical, R²³ representing a hydrogen atom or an alkyl or phenyl radical, and R²⁴ and R²⁵ representing independently alkyl radicals,
R₂₀ representing a hydrogen atom or an alkyl radical,
or also R¹⁹ and R²⁰ forming together with the nitrogen atom a heterocycle with 4 to 7 members containing 1 to 2 heteroatoms, the members necessary to complete the heterocycle being chosen independently from the —CR²⁶R²⁷—, —O—, —S— and —NR²⁸— radicals, R²⁶ and R²⁷ representing independently each time that they occur a hydrogen atom or an alkyl radical, and R²⁸ representing a hydrogen atom or an alkyl or aralkyl radical, or also R₂₈ representing a phenyl radical optionally substituted from 1 to 3 times by substituents chosen independently from a halogen atom and an alkyl or alkoxy radical,
R²¹ representing a hydrogen atom, an alkyl radical or an aralkyl radical the aryl group of which is optionally substituted from 1 to 3 times by substituents chosen independently from the group constituted by a halogen atom, an alkyl radical, a haloalkyl radical, an alkoxy radical, a haloalkoxy radical, a hydroxy radical, a nitro radical, a cyano radical, the phenyl radical, an SO₂NHR²⁹ radical and an NR³⁰R³¹ radical, R²⁹ representing a hydrogen atom or an alkyl or phenyl radical, and R³⁰ and R³¹ representing independently alkyl radicals,
R₂₂ representing a hydrogen atom or an alkyl radical, or also R²¹ and R²² forming together with the nitrogen atom a heterocycle with 4 to 7 members comprising 1 to 2 heteroatoms, the members necessary to complete the heterocycle being chosen independently from the —CR³²R³³—, —O—, —S— and —NR₃₄-radicals, R³² and R³³ representing independently each time that they occur a hydrogen atom or an alkyl radical, and R³⁴ representing a hydrogen atom, an alkyl or aralkyl radical, or also R³⁴ representing a phenyl radical optionally substituted from 1 to 3 times by substituents chosen independently from a halogen atom and an alkyl or alkoxy radical; and
W represents O or S;
or a pharmaceutically acceptable salt of a compound of general formula (III) defined above.

13. Product according to claim 12, characterized in that the Cdc25 phosphatase inhibitor is chosen from 5-{[2-(dimethylamino)ethyl]amino}-2-methyl-1,3-benzothiazole-4,7-dione and its pharmaceutically acceptable salts.

14. Product according to claim 4, characterized in that the other anticancer agent is a cycline dependent kinase (CDK) inhibitor and/or a glycogen synthase kinase-3 (GSK-3) inhibitor which responds to general formula (IV) 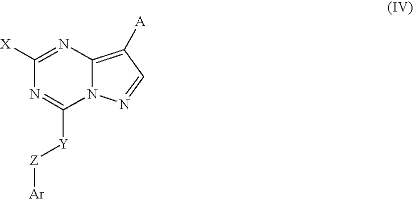
in the racemic form, enantiomeric form or in any combination of these forms, in which
A represents a hydrogen atom, a halogen atom, a formyl, cyano, nitro, guanidinoaminomethylenyl, (1,3-dihydro-2-oxoindol)-3-ylidenemethyl, alkylcarbonyl, aralkylcarbonyl or heteroaralkylcarbonyl radical, or also a -L-NR₁R² radical in which L represents an alkylene radical and R₁ and R² are chosen independently from a hydrogen atom and an alkyl radical or R₁ and R² taken together with the nitrogen atom which carries them form a heterocycle with 5 to 7 members, the complementary members being chosen independently from the group comprising —CH₂—, —NR₃—, —S— and —O—, R³ representing independently each time that it occurs a hydrogen atom or an alkyl radical;
X represents a hydrogen atom, an alkylthio, aralkylthio, alkylthioxo or aralkylthioxo radical, or also an NR⁴R⁵ radical in which R⁴ represents an alkyl radical, a hydroxyalkyl radical, a cycloalkyl radical optionally substituted by one or more radicals chosen from the alkyl, hydroxy and amino radicals, an aralkyl radical the aryl radical of which is optionally substituted by one or more radicals chosen from a halogen atom, the cyano radical, the nitro radical and the alkyl or alkoxy radicals, or also R⁴ represents a heteroaryl or heteroarylalkyl radical, the heteroaryl radical of the heteroaryl or heteroarylalkyl radicals being optionally substituted by one or more alkyl radicals and R⁵ represents a hydrogen atom, or then R⁴ and R⁵ taken together with the nitrogen atom which carries them form a heterocycle with 5 to 7 members, the complementary members being chosen independently from the group comprising —CH₂—, —NR⁶—, —S— and —O—, R⁶ representing independently each time that it occurs a hydrogen atom or an alkyl or hydroxyalkyl radical;
Y represents NH or an oxygen atom;
Z represents a bond or an alkyl or alkylthioalkyl radical; and
Ar represents a carbocyclic aryl radical optionally substituted from 1 to 3 times by radicals chosen independently from a halogen atom, the cyano radical, the nitro radical, an alkyl or alkoxy radical and an NR⁷R₈ radical in which R⁷ and R⁸ independently represent a hydrogen atom or an alkyl radical or R⁷ and R⁸ taken together with the nitrogen atom which carries them form a heterocycle with 5 to 7 members, the complementary members being chosen independently from the group comprising —CH₂—, —NR₉—, —S— and —O—, R⁹ representing independently each time that it occurs a hydrogen atom or an alkyl radical,
or also Ar represents a heterocyclic aryl radical containing 5 or 6 members and in which the heteroatom or heteroatoms are chosen from nitrogen, oxygen or sulphur atoms, said heteroatoms can optionally be oxidized and said heterocyclic aryl radical can optionally be substituted by one or more radicals chosen independently from the alkyl, aminoalkyl, alkylaminoalkyl and dialkylaminoalkyl radicals;
or a pharmaceutically acceptable salt of a compound of general formula (IV) defined above.

15. Product according to claim 14, characterized in that the CDK and/or GSK-3 inhibitor is chosen from 8-bromo-2-(1R-isopropyl-2-hydroxyethylamino)-4-(3-fluorophenylmethylamino)-pyrazolo[1,5-a]-1,3,5-triazine, 8-bromo-2-(1R-isopropyl-2-hydroxyethylamino)-4-(3-pyridylmethylamino)pyrazolo[1,5-a]-1,3,5-triazine and the pharmaceutically acceptable salts of the latter.

16. Product according to claim 4, characterized in that the other anticancer agent is a farnesyltransferase inhibitor which corresponds to general formula (V) 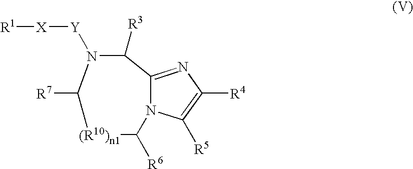
in which:
n1 represents 0 or 1;
X represents, independently each time that it occurs, (CHR¹¹)ₙ₃(CH₂)ₙ₄Z(CH₂)ₙ₅;
Z representing O, N(R₁²), S, or a bond;
n3 representing, independently each time that it occurs, 0 or 1;
each of n4 and n5 representing, independently each time that they occur, 0, 1, 2, or 3;
Y represents, independently each time that it occurs, CO, CH₂, CS, or a bond;
R¹ represents one of the radicals 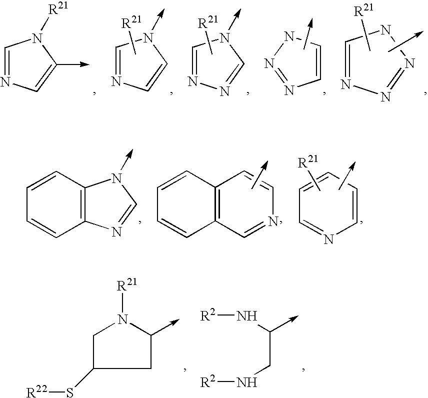
or N(R²⁴R²⁵) each of R², R₁, and R¹² representing, independently each time that it occurs, H or an optionally substituted radical chosen from the group consisting of a (C₁₋₆)alkyl radical and an aryl radical, said optionally substituted radical being optionally substituted by at least one radical chosen from the R⁸ and R³⁰ radicals, each substituent being chosen independently of the others;
R³ represents, independently each time that it occurs, H or an optionally substituted radical chosen from the group consisting of the (C₁₋₆)alkyl, (C₂₋₆)alkenyl, (C₂₋₆)alkynyl, (C₃₋₆)cycloalkyl, (C₃₋₆)cycloalkyl(C₁₋₆)alkyl, (C₅₋₇)cycloalkenyl, (C₅₋₇)cycloalkenyl(C₁₋₆)alkyl, aryl, aryl(C₁₋₆)alkyl, heterocyclyl, and heterocyclyl(C₁₋₆)alkyl radicals, said optionally substituted radical being optionally substituted by at least one radical chosen from the R³⁰ radicals, each substituent being chosen independently of the others;
each of R⁴ and R⁵ represents, independently each time that it occurs, H or an optionally substituted radical chosen from the group consisting of the (C₁₋₆)alkyl, (C₃₋₆)cycloalkyl, aryl and heterocyclyl radicals, said optionally substituted radical being optionally substituted by at least one radical chosen from the R³⁰ radicals, each substituent being chosen independently of the others, or R⁴ and R⁵ taken together with the carbon atoms to which they are attached together form an aryl radical;
R⁶ represents, independently each time that it occurs, H or an optionally substituted radical chosen from the group consisting of the (C₁₋₆)alkyl, (C₂₋₆)alkenyl, (C₃₋₆)cycloalkyl, (C₃₋₆)cycloalkyl(C₁₋₆)alkyl, (C₅₋₇)cycloalkenyl, (C₅₋₇)cycloalkenyl(C₁₋₆)alkyl, aryl, aryl(C₁₋₆)alkyl, heterocyclyl and heterocyclyl(C₁₋₆)alkyl radicals, said optionally substituted radical being optionally substituted by at least one radical chosen from the OH, (C₁₋₆)alkyl, (C₁₋₆)alkoxy, —N(R⁸R⁹), —COOH, —CON(R⁸R⁹) and halo radicals, each substituent being chosen independently of the others;
R⁷ represents, independently each time that it occurs, H, ═O, ═S, H or an optionally substituted radical chosen from the group consisting of the (C₁₋₆)alkyl, (C₂₋₆)alkenyl, (C₃₋₆)cycloalkyl, (C₃₋₆)cycloalkyl(C₁₋₆)alkyl, (C₅s₇)cycloalkenyl, (C₅₋₇)cycloalkenyl(C₁₋₆)alkyl, aryl, aryl(C₁₋₆)alkyl, heterocyclyl and heterocyclyl(C₁₋₆)alkyl radicals, said optionally substituted radical being optionally substituted by at least one radical chosen from the OH, (C₁₋₆)alkyl, (C₁₋₆)alkoxy, —N(R⁸R⁹), —COOH, —CON(R⁸R⁹) and halo radicals, each substituent being chosen independently of the others;
each of R⁸ and R⁹ representing, independently each time that it occurs, H, (C₁₋₆)alkyl, (C₂₋₆)alkenyl, (C₂₋₆)alkynyl, aryl, or aryl(C₁₋₆)alkyl;
R¹⁰ represents C;
or, when n1=0, R⁶ and R⁷ can be taken together with the carbon atoms to which they are attached to form an aryl or cyclohexyl radical;
R²¹ represents, independently each time that it occurs, H or an optionally substituted radical chosen from the group consisting of the (C₁₋₆)alkyl and aryl(C₁₋₆)alkyl radicals, said optionally substituted radical being optionally substituted by at least one radical chosen from the R⁸ and R³⁰ radicals, each substituent being chosen independently of the others;
R²² represents H, (C₁₋₆)alkylthio, (C₃₋₆)cycloalkylthio, R⁸—CO—, or a substituent of formula 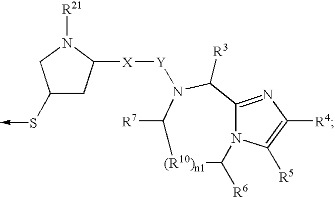
each of R²⁴ and R²⁵ represents, independently each time that it occurs, H, (C₁₋₆)alkyl or aryl(C₁₋₆)alkyl;
R³⁰ represents, independently each time that it occurs, (C₁₋₆)alkyl, —O—R⁸, —S(O),₆R⁸, —S(O),₇N(R⁸R⁹), —N(R⁸R⁹), —CN, —NO₂, —CO₂R₈, —CON(R⁸R⁹), —NCO—R⁸, or halogen,
each of n6 and n7 representing, independently each time that it occurs, 0, 1 or 2;
said heterocyclyl radical being azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothio-pyranyl sulphone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, 2-oxopiperazinyl, 2-oxopiperidinyl, 2-oxopyrrolidinyl, piperidyl, piperazinyl, pyridyl, pyridyl-N-oxide, quinoxalinyl, tetrahydrofuryl, tetrahydroisoquinolinyl, tetrahydroquinolinyl, thiamorpholinyl, thiamorpholinyl sulphoxide, thiazolyl, thiazolinyl, thienofuryl, thienothienyl or thienyl;
said aryl radical being phenyl or naphthyl;
it being understood that:
when n1=1, R₁₀ is C and R⁶ represents H, then R₁₀ and R⁷ can form, taken together, the radical 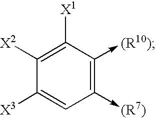
or when n1=1, R₁₀ is C, and R⁷ is ═O, —H, or ═S, then R₁₀ and R⁶ can form, taken together, the radical 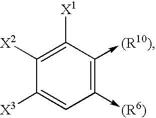
with each of X¹, X², and X³ representing, independently, H, a halogen atom, —NO₂, —NCO—R⁸, —CO₂R⁸, —CN, or —CON(R⁸R⁹); and
when R₁ is N(R²⁴R²⁵), then n3 represents 1, each of n4 and n5 represents 0, Z is a bond, and R³ and R¹¹ can form, taken together, the radical 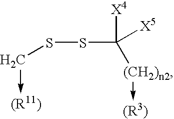
with n2 representing an integer from 1 to 6, and each of X⁴ and X⁵ representing, independently, H, (C₁₋₆)alkyl or aryl, or X⁴ and X⁵ forming, taken together, a (C₃₋₆)cycloalkyl radical;
or a pharmaceutically acceptable salt of a compound of general formula (V) defined above.

17. Product according to claim 16, characterized in that the farnesyltransferase inhibitor is chosen from 1-(2-(1-((4-cyano)phenylmethyl)imidazol-4-yl)-1-oxoethyl-2,5-dihydro-4-(2-methoxyphenyl)imidazo[1,2c][1,4]benzodiazepine and 4-(2-bromophenyl)-1-(2-(1-((4-cyano-3-methoxy)phenylmethyl)-imidazo-5-yl)-1-oxoethyl)-1,2-dihydro-8-fluoro-imidazo[1,2a][1,4]-benzodiazepine, and the pharmaceutically acceptable salts of these compounds.

18. Product according to claim 4, characterized in that the other anticancer agent is chosen from 7-(2-amino-1-oxo-3-thiopropyl)-8-(cyclohexylmethyl)-2-phenyl-5,6,7,8-tetrahydroimidazo[1,2a]pyrazine, cisplatin, 8-bromo-2-(1R-isopropyl-2-hydroxyethylamino)-4-(3-pyridylmethylamino)pyrazolo[1,5-a]-1,3,5-triazine, 8-bromo-2-(1R-isopropyl-2-hydroxyethylamino)-4-(3-fluorophenylmethylamino)-pyrazolo[1,5-a]-1,3,5-triazine, 1-(2-(1-((4-cyano)phenylmethyl)imidazol-4-yl)-1-oxoethyl-2,5-dihydro-4-(2-methoxyphenyl)imidazo[1,2c][1,4]benzodiazepine, 4-(2-bromophenyl)-1-(2-(1-((4-cyano-3-methoxy)phenylmethyl)-imidazo-5-yl)-1-oxoethyl)-1,2-dihydro-8-fluoro-imidazo[1,2a][1,4]-benzodiazepine and their pharmaceutically acceptable salts.

19. Product according to claim 18, characterized in that the other anticancer agent is chosen from 7-(2-amino-1-oxo-3-thiopropyl)-8-(cyclohexylmethyl)-2-phenyl-5,6,7,8-tetrahydroimidazo[1,2a]pyrazine and the cisplatin.

20. Product according to one of the claims 1 to 19, characterized in that it comprises an analogue of mikanolide or dihydromikanolide chosen from the compounds which correspond to general formula (I): 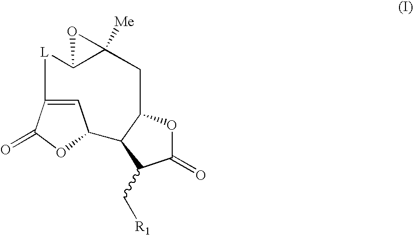
corresponding to general sub-formulae (I)₁ and (I)₂ 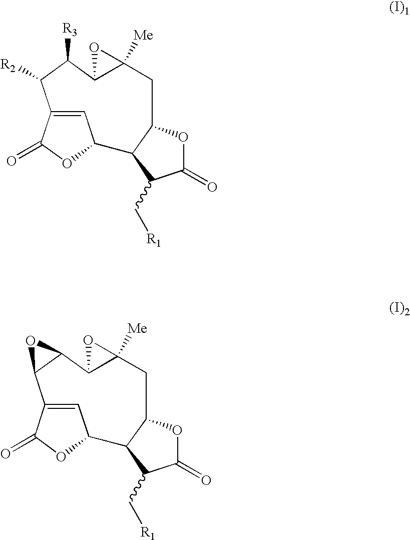
in which
R₁ represents a hydrogen atom or an SR₄ or NR₄R₅ radical;
R₂ represents SR₆ or NR₆R₇;
R₃ represents OH, O(CO)R₁₄, OSiR₁₅R₁₆R₁₇, O(CO)OR₁₈ or O(CO)NHR₁₈;
R₄ and R₆ represent, independently, an alkyl radical, a cycloalkyl, cycloalkylalkyl, hydroxyalkyl radical or also one of the aryl or aralkyl radicals optionally substituted on their aryl group by one or more radicals chosen from the alkyl, hydroxy or alkoxy radicals,
R₅ and R₇ represent, independently, a hydrogen atom, an alkyl radical, a cycloalkyl, cycloalkylalkyl, hydroxyalkyl radical or also one of the aryl or aralkyl radicals optionally substituted on their aryl group by one or more radicals chosen from the alkyl, hydroxy or alkoxy radicals,
R₄ and R₅ being able to form together with the nitrogen atom which carries them a heterocycle with 5 to 7 members, the complementary members being chosen from the —CR₈R₉—, —NR₁₀-, —O— and —S— radicals, it being understood however that there can only be a single member chosen from —O— or —S— in said heterocycle, and R₆ and R₇ being able to form together with the nitrogen atom which carries them a heterocycle with 5 to 7 members, the complementary members being chosen from the —CR₁₁R₁₂—, —NR₁₃—, —O— and —S— radicals, it being understood however that there can be only a single member chosen from —O— or —S— in said heterocycle,
R₈, R₁₀, R₁, and R₁₃ represent, independently each time that they occur, a hydrogen atom or an alkyl, alkoxycarbonyl or aralkyl radical,
R₉ and R₁₂ representing, independently each time that they occur, a hydrogen atom or each of R₉ and R₁₂ can form with R₈ and R₁, respectively an —O—(CH₂)₂—O— radical attached at both ends to the carbon atom which carries them, such a radical only being present a maximum of once per NR₄R₅ or NR₆R₇ radical, represent, independently each time that they occur, a hydrogen atom or an alkyl radical;
R₁₄ represents an alkyl, cycloalkyl or adamantyl radical or one of the aryl, heteroaryl, aralkyl or heteroaralkyl radicals optionally substituted on their aryl or heteroaryl group by one or more radicals chosen from a halogen atom and the alkyl, haloalkyl, nitro, hydroxy, alkoxy, alkylthio or phenyl radicals, or also R₁₄ is such that R₁₄—COOH represents a natural amino acid or the optical enantiomer of such an amino acid;
R₁₅, R₁₆ and R₁₇ represent, independently, an alkyl radical or a phenyl radical;
R₁₈ represents an alkyl, cycloalkyl or adamantyl radical or one of the aryl, heteroaryl, aralkyl or heteroaralkyl radicals optionally substituted on their aryl or heteroaryl group by one or more radicals chosen from a halogen atom and the alkyl, haloalkyl, nitro, hydroxy, alkoxy, alkylthio or phenyl radicals;
it being understood however that when the compounds correspond to general sub-formula (1)₂, then R₁ does not represent a hydrogen atom;
or is a pharmaceutically acceptable salt of a compound of general formula (I).

21. Product according to claim 20, characterized in that the analogue of mikanolide or dihydromikanolide is chosen from:
12-diisopropylaminomethyl-7-methyl-3,6,10,15-tetraoxapentacyclo [12.2.1.0^{2,4}.0^{5,7}.0^{9,13}]heptadec-1(17)-ene-11,16-dione;
12-dimethylamino-3-dimethylaminomethyl-11-hydroxy-8-methyl-5,9,15-trioxatetracyclo[11.2.1.0^{2,6}.0^{8,10}]hexadec-13(16)-ene-4,14-dione;
12-benzyl(methyl)amino-3-benzyl(methyl)aminomethyl-11-hydroxy-8-methyl-5,9,15-trioxatetracyclo[11.2.1.0^{2,6}.0^{8,10}]hexadec-13(16)-ene-4,14-dione;
11-hydroxy-8-methyl-12-morpholino-3-morpholinomethyl-5,9,15-trioxatetracyclo[11.2.1.0^{2,6}.0^{8,10}]hexadec-13(16)-ene-4,14-dione;
12-dimethylamino-11-hydroxy-3,8-dimethyl-5,9,15-trioxatetracyclo[11.2.1.0^{2,6}.0^{8,10}]hexadec-13(16)-ene-4,14-dione;
11-hydroxy-3,8-dimethyl-12-(4-methylpiperidino)-5,9,15-trioxatetracyclo[11.2.1.0^{2,6}.0^{8,10}]hexadec-13(16)-ene-4,14-dione;
11-hydroxy-3,8-dimethyl-12-pyrrolidino-5,9,15-trioxatetracyclo[11.2.1.0^{2,6}.0^{8,10}]hexadec-13(16)-ene-4,14-dione;
ethyl 1-[11-hydroxy-3,8-dimethyl-4,14-dioxo-5,9,15-trioxatetracyclo [11.2.1.0^{2,6}.0^{8,10}]hexadec-13(16)-en-12-yl]4-piperidinecarboxylate;
12-(4-benzylpiperidino)-11-hydroxy-3,8-dimethyl-5,9,15-trioxatetracyclo[11.2.1.0^{2,6}.0^{8,10}]hexadec-13(16)-ene-4,14-dione;
11-hydroxy-3,8-dimethyl-12-piperidino-5,9,15-trioxatetracyclo[11.2.1.0^{2,6}.0^{8,10}]hexadec-13(16)-ene-4,14-dione;
12-(1,4-dioxa-8-azaspiro[4.5]dec-8-yl)-11-hydroxy-3,8-dimethyl-5,9,15-trioxatetracyclo[11.2.1.0^{2,6}.0^{8,10}]hexadec-13(16)-ene-4,14-dione;
11-hydroxy-3,8-dimethyl-12-morpholino-5,9,15-trioxatetracyclo[11.2.1.0^{2,6}.0^{8,10}]hexadec-13(16)-ene-4,14-dione;
11-(tert-butyldimethylsiloxy)-12-dimethylamino-3,8-dimethyl-5,9,15-trioxatetracyclo[11.2.1.0^{2,6}.0^{8,10}]hexadec-13(16)-ene-4,14-dione;
3,8-dimethyl-12-(4-methylpiperidino)-4,14-dioxo-5,9,15-trioxatetracyclo[11.2.1.0^{2,6}.0^{8,10}]hexadec-13(16)-en-11-yl acetate;
3,8-dimethyl-12-(4-methylpiperidino)-4,14-dioxo-111-phenylcarbonyloxy-5,9,15-trioxatetracyclo[11.2.1.0^{2,6}.0^{8,10}]hexadec-13(16)-ene;
ethyl 3,8-dimethyl-12-(4-methylpiperidino)-4,14-dioxo-5,9,15-trioxatetracyclo[11.2.1.0^{2,6}.0^{8,10}]hexadec-13(16)-en-11-ylcarbonate;
11-hydroxy-12-isobutylsulphanyl-3-isobutylsulphanylmethyl-8-methyl-5,9,15-trioxatetracyclo[11.2.1.0^{2,6}.0^{8,10}]hexadec-13(16)-ene-4,14-dione;
11-hydroxy-12-isobutylsulphanyl-3,8-dimethyl-5,9,15-trioxatetracyclo[11.2.1.0^{2,6}.0^{8,10}]hexadec-13(16)-ene-4,14-dione;
12-(dimethylamino)-3,8-dimethyl-4,14-dioxo-5,9,15-trioxatetracyclo[11.2.1.0^{2,6}.0^{8,10}]hexadec-13(16)-en-11-yl benzoate;
12-(dimethylamino)-3,8-dimethyl-4,14-dioxo-5,9,15-trioxatetracyclo[11.2.1.0^{2,6}.0^{8,10}]hexadec-13(16)-en-11-yl acetate;
12-(dimethylamino)-3,8-dimethyl-4,14-dioxo-5,9,15-trioxatetracyclo [11.2.1.0^{2,6}.0^{8,10}]hexadec-13(16)-en-11-yl cyclohexanecarboxylate;
12-(dimethylamino)-3,8-dimethyl-4,14-dioxo-5,9,15-trioxatetracyclo [11.2.1.0^{2,6}.0^{8,10}]hexadec-13(16)-en-I 1-yl 4-fluorobenzoate f;
12-(dimethylamino)-3,8-dimethyl-4,14-dioxo-5,9,15-trioxatetracyclo [11.2.1.0^{2,6}.0^{8,10}]hexadec-13(16)-en-11-yl heptanoate;
12-(dimethylamino)-3,8-dimethyl-4,14-dioxo-5,9,15-trioxatetracyclo [11.2.1.0^{2,6}.0^{8,10}]hexadec-13(16)-en-11-yl 4-(trifluoromethyl)benzoate;
12-(dimethylamino)-3,8-dimethyl-4,14-dioxo-5,9,15-trioxatetracyclo [11.2.1.0^{2,6}.0^{8,10}]hexadec-13(16)-en-11-yl 2-thiophenecarboxylate;
12-(dimethylamino)-3,8-dimethyl-4,14-dioxo-5,9,15-trioxatetracyclo [11.2.1.0^{2,6}.0^{8,10}]hexadec-13(16)-en-11-yl 3-dimethylbutanoate;
12-(dimethylamino)-3,8-dimethyl-4,14-dioxo-5,9,15-trioxatetracyclo [11.2.1.0^{2,6}.0^{8,10}]hexadec-13(16)-en-11-yl 1-benzothiophene-2-carboxylate;
12-(dimethylamino)-3,8-dimethyl-4,14-dioxo-5,9,15-trioxatetracyclo [11.2.1.0^{2,6}.0^{8,10}]hexadec-13(16)-en-11-yl 2-furoate;
12-(dimethylamino)-3,8-dimethyl-4,14-dioxo-5,9,15-trioxatetracyclo [11.2.1.0^{2,6}.0^{8,10}]hexadec-13(16)-en-11-yl 5-nitro-2-furoate;
12-(dimethylamino)-3,8-dimethyl-4,14-dioxo-5,9,15-trioxatetracyclo[11.2.1.0^{2,6}.0^{8,10}]hexadec-13(16)-en-11-yl 2-thienylacetate;
12-(dimethylamino)-3,8-dimethyl-4,14-dioxo-5,9,15-trioxatetracyclo[11.2.1.0^{2,6}.0^{8,10}]hexadec-13(16)-en-11-yl phenoxyacetate;
8-(dimethylamino)-3,10a-dimethyl-2,6-dioxodecahydro-4,7-methenofuro [3,2-c]oxireno[/]oxacycloundecin-9-yl 4-tert-butylphenylcarbamate;
8-(dimethylamino)-3,10a-dimethyl-2,6-dioxodecahydro-4,7-methenofuro [3,2-c]oxireno[f]oxacycloundecin-9-yl thien-2-ylcarbamate of;
8-(dimethylamino)-3,10a-dimethyl-2,6-dioxodecahydro-4,7-methenofuro [3,2-c]oxireno[/]oxacycloundecin-9-yl 2-methoxyphenylcarbamate;
8-(dimethylamino)-3,10a-dimethyl-2,6-dioxodecahydro-4,7-methenofuro [3,2-c]oxireno[f]oxacycloundecin-9-yl 2(methylthio)phenylcarbamate;
8-(dimethylamino)-3,10a-dimethyl-2,6-dioxodecahydro-4,7-methenofuro [3,2-c]oxireno[f]oxacycloundecin-9-yl 2-ethoxyphenylcarbamate;
8-(dimethylamino)-3,10a-dimethyl-2,6-dioxodecahydro-4,7-methenofuro [3,2-c]oxireno[f]oxacycloundecin-9-yl thien-3-ylcarbamate;
8-(dimethylamino)-3,10a-dimethyl-2,6-dioxodecahydro-4,7-methenofuro [3,2-c]oxireno[f]oxacycloundecin-9-yl 1-benzothien-3-ylcarbamate;
8-(dimethylamino)-3,10a-dimethyl-2,6-dioxodecahydro-4,7-methenofuro [3,2-c]oxireno[/]oxacycloundecin-9-yl N-(ter-butoxycarbonyl)glycinate;
8-(dimethylamino)-3,10a-dimethyl-2,6-dioxodecahydro-4,7-methenofuro [3,2-c]oxireno[/]oxacycloundecin-9-yl thien-3-ylacetate;
8-(dimethylamino)-3,10a-dimethyl-2,6-dioxodecahydro-4,7-methenofuro [3,2-c]oxireno[/]oxacycloundecin-9-yl 1-benzothien-3-ylacetate;
8-(dimethylamino)-3,10a-dimethyl-2,6-dioxodecahydro-4,7-methenofuro [3,2-c]oxireno[f]oxacycloundecin-9-yl thiophene-3-carboxylate;
8-(dimethylamino)-3,10a-dimethyl-2,6-dioxodecahydro-4,7-methenofuro [3,2-c]oxireno[A]oxacycloundecin-9-yl 5-phenylthien-2-ylcarbamate;
8-(dimethylamino)-3,10a-dimethyl-2,6-dioxodecahydro-4,7-methenofuro [3,2-c]oxireno[f]oxacycloundecin-9-yl 1-adamantylcarbamate;
8-(dimethylamino)-3,10a-dimethyl-2,6-dioxodecahydro-4,7-methenofuro [3,2-c]oxireno[f]oxacycloundecin-9-yl 2-naphthylcarbamate;
-(dimethylamino)-3,10a-dimethyl-2,6-dioxodecahydro-4,7-metheno-furo [3,2-c]oxireno[/]oxacycloundecin-9-yl 2-tert-butyl-6-methylphenylcarbamate;
8-(dimethylamino)-3,10a-dimethyl-2,6-dioxodecahydro-4,7-metheno-furo [3,2-c]oxireno[f]oxacycloundecin-9-yl 2,5-dimethoxyphenylcarbamate;
and the pharmaceutically acceptable salts of the latter.

22. Product according to claim 21, characterized in that the analogue of mikanolide or dihydromikanolide is 8-(dimethylamino)-3,10a-dimethyl-2,6-dioxodecahydro-4,7-methenofuro[3,2-c]oxireno[f]oxacycloundecin-9-yl 2-naphthylcarbamate, and its pharmaceutically acceptable salts.

23. Product according to one of claims 1 to 19, characterized in that it comprises dihydromikanolide.

24. Product according to one of the claims 1 to 23, characterized in that the treatment is aimed at a cancer chosen from cancers of the oesophagus, the stomach, the intestines, the rectum, the oral cavity, the pharynx, the larynx, the lung, the colon, the breast, the cervix uteri, the corpus endometrium, the ovaries, the prostate, the testicles, the bladder, the kidneys, the liver, the pancreas, the bones, the connective tissue, the skin, the eyes, the brain and the central nervous system, as well as cancer of the thyroid, leukemia, Hodgkin's disease, lymphomas other that those related to Hodgkin's and multiple myelomas.

25. Pharmaceutical composition comprising, as active ingredient, a product according to one of claims 1 to 24.
